(19) United States
(12) Patent Application Publication
(10) Pub. No.: US 2024/0182920 A1
(43) Pub. Date: Jun. 6, 2024

(54) METHOD FOR TRANSDUCING CELLS WITH VIRAL VECTOR

(71) Applicant: CARSGEN THERAPEUTICS CO., LTD., Shanghai (CN)

(72) Inventors: Huamao WANG, Shanghai (CN); Huiping GAO, Shanghai (CN); Xiao TONG, Shanghai (CN); Yefeng YAO, Shanghai (CN); Yinyu ZHU, Shanghai (CN); Zonghai LI, Shanghai (CN)

(21) Appl. No.: 17/759,294

(22) PCT Filed: Jan. 22, 2021
(86) PCT No.: PCT/CN2021/073418

§ 371 Date: Jul. 22, 2022

(30) Foreign Application Priority Data
Jan. 22, 2020 (CN) 202010075423.5
Jan. 20, 2021 (CN) 202110076311.6

(51) Int. Cl. C12N 15/86; A61K 35/17; A61K 39/00; A61P 35/00; C12N 5/078

(52) U.S. Cl. CPC C12N 15/86; A61K 35/17; A61K 39/4611; A61P 35/00; C12N 5/0634; C12N 2510/00; C12N 2740/15043

(57) ABSTRACT

Provided is a method for transducing cells with a viral vector, and further provided are cells obtained through recombination or heterologous gene transduction and compositions thereof, and a method for using same in adoptive immunotherapy. Under the premise of not affecting the expression of recombinant nucleic acid, the method shortens the activation and transduction time during the preparation process of genetically engineered cells.

### TECHNICAL FIELD

The invention belongs to the field of genetic engineering, and in particular, relates to a method for tranferring a recombinant nucleic acid into cells through virus transduction.

### BACKGROUND

The role of immune effector cells (such as T cells, NK cells, NK T cells, etc.) in tumor immunotherapy attracted increasing attention. In recent years, people have modified immune effector cells with exogenous receptors to obtain T cells specifically recognizing tumor-related antigens, for carrying out tumor therapy, such as chimeric antigen receptor-modified CAR T cells and chimeric TCR receptor-modified TCR T cells, etc.

Generally, cells that recognize tumor-associated antigens are obtained by introducing a recombinant nucleic acid encoding an exogenous receptor that recognizes tumor-associated antigens into a viral vector, and then infecting and transducing cells with the viral vector carrying the recombinant nucleic acid. However, since it usually takes a long time for viral vectors carrying recombinant nucleic acids to infect and transduce cells (such as the preparation of CAR T cells), in the conventional process of transduction of exogenous nucleic acids into T cells by viral vectors, it usually takes more than one day for activating T cells, and then the viral transduction will take 1 day. After the transduction is completed, the cells are required to be amplified, which takes 1-2 weeks. Therefore, it takes a long time for the preparation of CAR T cells, which will not only increase the time cost and reagent cost for the preparation of cell products, but also increase the risk of cell mutation. When cell therapy products are given to patients, some patients have already experienced tumor progression, which will delay the timing of tumor treatment and affect effects of clinical treatment.

### SUMMARY OF THE INVENTION

The purpose of the present invention is to provide a method for transducing cells with a viral vector, which can significantly shorten the preparation time of receptor-modified cells that recognize tumor-associated antigens, without affecting but even further enhancing the efficacy of cell therapy.

In the first aspect, the present invention provides a method for transducing cells with a viral vector, the method comprising:
Step (1), co-incubating an input composition comprising cells to be transduced, a stimulator for the cells to be transduced, and viral vector particles carrying a recombinant nucleic acid for an incubation time of no more than 72 hours,
Step (2), harvesting and obtaining an output composition comprising cells transduced with the recombinant nucleic acid;
preferably, the incubation time is 1 hour to 72 hours;
more preferably, the incubation time is 2 hours-48 hours;
more preferably, the incubation time is 2 hours-36 hours;
more preferably, the incubation time is 12 hours-36 hours;
more preferably, the incubation time is 12 hours-24 hours;
more preferably, the incubation time is 15 hours to 24 hours.

In the second aspect, the present invention provides a method for transducing cells with a viral vector, the method comprising:
Step (1), incubating an input composition comprising cells to be transduced and a stimulator for the cells to be transduced for an incubation time of no more than 72 h,
step (2), adding and incubating viral vector particles carrying a recombinant nucleic acid for an incubation time of not more than 24 hours,
Step (3), harvesting and obtaining an output composition comprising cells transduced with the recombinant nucleic acid;

preferably, the total incubation time of steps (1) and (2) is no more than 72 hours.

In a specific embodiment, the total incubation time of steps (1) and (2) is no more than 60 hours, or no more than 48 hours, or no more than 32 hours, or no more than 24 hours.

In a specific embodiment, the incubation time of step (1) is 2-72 hours;
preferably, the incubation time in step (1) is 2-71 hours;
more preferably, the incubation time of step (1) is 2-48 hours;
more preferably, the incubation time of step (1) is 2-32 hours;
more preferably, the incubation time of step (1) is 2-28 hours;
more preferably, the incubation time of step (1) is 3-24 hours;
more preferably, the incubation time of step (1) is 5-24 hours;
more preferably, the incubation time of step (1) is 7-24 hours;
more preferably, the incubation time of step (1) is 7-23 hours;
more preferably, the incubation time of step (1) is 10-23 hours;
more preferably, the incubation time of step (1) is 15-23 hours;
more preferably, the incubation time of step (1) is 15-22 hours.

In a specific embodiment, the incubation time in step (2) is 30 mins-24 hours;
preferably, the incubation time of step (2) is 30 mins-21 hours;
preferably, the incubation time of step (2) is 30 mins-17 hours;
preferably, the incubation time of step (2) is 30 mins-12 hours;
preferably, the incubation time of step (2) is 30 mins-10 hours;
preferably, the incubation time of step (2) is 30 mins-8 hours;
preferably, the incubation time of step (2) is 1 hours-8 hours;
preferably, the incubation time of step (2) is 1 hours-4 hours;
more preferably, the incubation time of step (2) is 1 hour-3 hours.

In a specific embodiment, the input composition is obtained from peripheral blood, cord blood, bone marrow and/or induced pluripotent stem cells; preferably, the input composition is a leukopheresis sample; and preferably, the input composition is enriched or isolated CD3+ T cells, enriched or isolated CD4+ T cells or enriched or isolated CD8+ T cells or a combination thereof.

In a specific embodiment, the viral vector particle is derived from a retroviral vector; and preferably, the viral vector particle is a lentiviral vector.

In a specific embodiment, the multiplicity of infection of the viral vector particles is not higher than 20; preferably, the multiplicity of infection is 0.5-20; more preferably, the multiplicity of infection is 1.5-20; more preferably, the multiplicity of infection is 3-20; and more preferably, the multiplicity of infection is 3-12.

In a specific embodiment, the number of cells to be transduced in the input composition is not higher than 1*10¹⁰;
preferably, the number of cells to be transduced in the input composition is not less than 1*10⁵;
and more preferably, the number of cells to be transduced in the input composition is not less than 1*10⁶.

In a specific embodiment, the recombinant nucleic acid can encode a receptor that recognizes a specific target antigen; and preferably, the receptor that recognizes a specific target antigen is T cell receptor (TCR), chimeric antigen receptor (CAR), chimeric T cell receptor, or T cell antigen coupler (TAC).

In a specific embodiment, the chimeric antigen receptor (CAR) comprises a cell surface antigen recognition domain that specifically binds to a target antigen and an intracellular signaling domain comprising an ITAM.

In a preferred embodiment, the intracellular signaling domain comprises the intracellular domain of the CD3-zeta (CD3ζ) chain.

In a preferred embodiment, the chimeric antigen receptor (CAR) further comprises a transmembrane domain connecting the extracellular domain and the intracellular signaling domain.

In a preferred embodiment, the transmembrane domain comprises the transmembrane portion of CD28 and/or CD8.

In a preferred embodiment, the intracellular signaling domain also comprises the intracellular signaling domain of a T cell costimulatory molecule.

In a preferred embodiment, the T cell costimulatory molecules are selected from CD28 and/or 41BB.

In a preferred embodiment, the specific target antigen is a disease-associated antigen or a universal tag;
preferably, the disease is a cancer, autoimmune disease, or infectious disease;
preferably, the cancer is a hematological tumor; and more preferably, the hematological tumor is a leukemia, myeloma, lymphoma and/or a combination thereof.

In a specific embodiment, the specific target antigen is a tumor-associated antigen; preferably, the tumor-associated antigen is selected from: B cell maturation antigen (BCMA), carbonic anhydrase 9 (CAIX), EGFR, Her2/neu (receptor tyrosine kinase erbB2), CD19, CD20, CD22, mesothelin, CEA, CD23, CD24, CD30, CD33, CD38, CD44, EGFR, Epiglin 2 (EPG-2), Epiglin 40 (EPG-40), EPHa2, erb-B2, erb-B3, erb-B4, erbB dimer, EGFR vIII, folic acid Binding protein (FBP), FCRL5, FCRH5, fetal acetylcholine receptor, GD2, GD3, HMW-MAA, IL-22R-α, IL-13R-α2, kinase insertion domain receptor (kdr), L1 cell adhesion molecule (L1-CAM), melanoma-associated antigen (MAGE), TAG72, B7-H6, IL-13 receptor alpha 2 (IL-13Ra2), CA9, GD3, HMW-MAA, CD171, G250/CAIX, HLA-AI MAGEA1, HLA-A2, PSCA, folate receptor, CD44v6, CD44v7/8, avb6 integrin, 8H9, NCAM, VEGF receptor, 5T4, fetal AchR, NKG2D ligand, CD44v6, mesothelin, mucin 1 (MUC1), MUC16, PSCA, NKG2D, NY-ESO-1, MART-1, gp100, carcinoembryonic antigen, G protein-coupled receptor 5D (GPCR5D), ROR1, TAG72, VEGF-R2, carcinoembryonic antigen (CEA), prostate specific antigen, PSMA, ephrin B2, CD123, c-Met, GD-2, O-acetylated GD2 (OGD2), CE7, Wilms tumor 1 (WT-1), Cyclin, CCL-1, CD138, Claudin18.2, GPC3.

In a specific embodiment, the stimulator for the cells to be transduced is capable of activating one or more intracellular signaling domains of one or more components of a TCR complex or one or more intracellular signaling domains of one or more costimulatory molecules;
and preferably, the stimulator for the cells to be transduced comprises (i) a primary agent that specifically binds to a member of the TCR complex, optionally CD3, and (ii) a secondary agent that specifically binds to the T cell costimulatory molecule, optionally wherein the costimulatory molecule is selected from CD28, CD137(4-1-BB), OX40 or ICOS.

In a specific embodiment, the cells to be transduced are immune effector cells;
preferably, the cells to be transduced are T cells, NK cells, NKT cells, dendritic cells, macrophages, CIK cells, and stem cell-derived immune effector cells or a combination thereof;
and more preferably, the cells to be transduced are T cells.

In a preferred embodiment, the T cells are CD4+ and/or CD8+ cells.

In a preferred embodiment, the ratio of the CD4+ cells to the CD8+ cells is 1:1, 1:2, 2:1, 1:3, 3:1, 1:4, 4:1, 1:5, 5:1, 1:6 or 6:1.

In a preferred embodiment, reagent 4 for the selection or enrichment is included.

In a preferred embodiment, the reagent 4 that does not bind to the T cell can be removed by centrifugation.

In a preferred embodiment, the 4 is immobilized on a solid support, and preferably the solid support is a polymer matrix.

In a preferred embodiment, the polymer matrix is a polymer nanomatrix and/or bead reagent.

In a preferred embodiment, the bead reagent comprises a magnetic bead and/or microbead.

In a preferred embodiment, the activation and/or amplification is performed in vivo.

In a preferred embodiment, the sample is a leukapheresis sample.

In a preferred embodiment, the T cells are enriched or isolated CD3+ T cells, enriched or isolated CD4+ T cells, or enriched or isolated CD8+ T cells.

In a preferred embodiment, the T cells have been selected or enriched from a sample of a subject.

In a specific embodiment, the stimulator for the cells to be transduced comprises a CD3 binding molecule, a CD28 binding molecule, recombinant IL-2, recombinant IL-15, recombinant IL-7, recombinant IL-21 or a combination thereof;
and preferably, the stimulator for the cells to be transduced comprises an anti-CD3 antibody and/or an anti-CD28 antibody.

In a specific embodiment, the stimulator for the cells to be transduced can be removed by centrifugation prior to harvesting.

In a specific embodiment, the stimulator for the cells to be transduced is a free molecule.

In a specific embodiment, the stimulator for the cells to be transduced is immobilized on a solid support;
preferably, the solid support is a polymer matrix material;
and more preferably, the polymer matrix material is a degradable polymer nanomatrix or bead reagent.

In a specific embodiment, the bead reagent is a magnetic bead or microbead.

In a specific embodiment, the content of cells transduced with the recombinant nucleic acid in the output composition is not less than 30%, or not less than 40%, or not less than 50%, or not less than 60%, or not less than 70%, or not less than 80%.

In a specific embodiment, the content of cells transduced with the recombinant nucleic acid in the output composition is not higher than 50%; preferably, not higher than 40%, more preferably, not higher than 38%; more preferably, not higher than 35%; more preferably, not higher than 30%.

In a specific embodiment, compared with the content of naive cells in the cells to be transduced, the content of naive cells in the cells transduced with the recombinant nucleic acid is reduced;
preferably, the content of naive cells is reduced to less than 10%;
and more preferably, the content of naive cells is reduced to less than 5%.

In a specific embodiment, compared with the content of memory cells in the cells to be transduced, the content of memory cells in the cells transduced with the recombinant nucleic acid is increased;
preferably, the memory cells are memory stem cells;
and more preferably, the memory stem cells are TSCMs.

In a specific embodiment, the content of memory stem cells in the cells transduced with recombinant nucleic acid is about 2 times or more the content of memory stem cells in the cells to be transduced. Preferably, the content of memory stem cells in the cells transduced with recombinant nucleic acid is about 3 times or more the content of memory stem cells in the cells to be transduced.

In a specific embodiment, the cells transduced with the recombinant nucleic acid contain undifferentiated cells.

In specific embodiments, the input composition comprises recombinant IL-2, optionally recombinant human IL-2, at a concentration of 10 IU/mL to 500 IU/mL, 50 IU/mL to 250 IU/mL or 100 IU/mL to 200 IU/mL; or at a concentration of at least 10 IU/mL, 50 IU/mL, 100 IU/mL, 200 IU/mL, 300 IU/mL, 400 IU/mL, or 500 IU/mL; and/or
the input composition comprises recombinant IL-15, optionally recombinant human IL-15, at a concentration of 1 IU/mL to 100 IU/mL, 2 IU/mL to 50 IU/mL, or 5 IU/mL to 10 IU/mL; or at a concentration of at least 1 IU/mL, 2 IU/mL, 5 IU/mL, 10 IU/mL, 25 IU/mL, or 50 IU/mL; and/or
the input composition comprises recombinant IL-7, optionally recombinant human IL-7, at a concentration of 50 IU/mL to 1500 IU/mL, 100 IU/mL to 1000 IU/mL to 200 IU/mL to 600 IU/mL; or at a concentration of at least 50 IU/mL, 100 IU/mL, 200 IU/mL, 300 IU/mL, 400 IU/mL, 500 IU/mL, 600 IU/mL, 700 IU/mL, 800 IU/mL, 900 IU/mL, or 1000 IU/mL.

In a specific embodiment, the harvested output composition is washed to obtain the cells transduced with recombinant nucleic acid.

In a specific embodiment, the cells transduced with the recombinant nucleic acid are added to a buffer for preservation; and preferably, the buffer contains a cell cryopreservation agent.

In a specific embodiment, the cells transduced with the recombinant nucleic acid are harvested and administerrd to a subject in need thereof without in vitro expansion.

In the third aspect, the present invention provides a composition of the cells transduced with the recombinant nucleic acid produced by the method of the first or second aspect.

In a specific embodiment, the cells are immune effector cells.

In a specific embodiment, the cells are T cells.

In a specific embodiment, the proportion of TSCMs in the cells transduced with the recombinant nucleic acid is higher than the proportion of TSCMs in the cells to be transduced;
preferably, the proportion of TSCMs in the cells transduced with the recombinant nucleic acid is about 2 times or more the proportion of TSCMs in the cells to be transduced;
and more preferably, the proportion of TSCMs in the cells transduced with the recombinant nucleic acid is about 3 times or more the proportion of TSCM in the cells to be transduced.

In a specific embodiment, the proportion of TSCMs in the cells transduced with the recombinant nucleic acid is 10% or higher, preferably 13% or higher, and more preferably 15% or higher.

In a specific embodiment, the cells transduced with the recombinant nucleic acid are administered to a subject without in vitro expansion.

In the fourth aspect, the present invention provides a composition comprising the cells transduced with the recombinant nucleic acid of the third aspect and a pharmaceutically acceptable carrier.

In the fifth aspect, the present invention provides a method for adoptive cell therapy, comprising administering the composition of the fourth aspect to a subject in need thereof.

### TECHNICAL EFFECTS OF THE INVENTION

According to the present invention, not only the activation and/or activation steps before exposure to the retroviral vector particles can be shortened, but also the incubation time after transduction can be further shortened, in vitro activation, transduction and culture time can be even shortened to 1-2 days, and after activation and transduction is completed, the prepared cells can be used to treat patients without expansion. Under the same condition of activation, the later the lentiviral vector was added, the higher the transduction efficiency, although the actual transduction duration decreased.

It should be understood that the above-mentioned technical features of the present invention and the technical features specifically described in the following (e.g., embodiments) can be combined with each other to constitute new or preferred technical solutions, which fall within the scope of the present invention and are not repeated herein due to the limitation on the contents.

### DESCRIPTION OF DRAWINGS

FIG. 1. Changes in transduction efficiency of CAR T cells prepared by the new process and conventional controls at different times after transduction.

FIG. 2. Changes in transduction efficiency of CAR T cells prepared by the new process and conventional controls at different times after transduction, under various activation and transduction conditions.

FIG. 3. Changes in transduction efficiency of CAR T cells prepared by the new process and conventional controls at different times after transduction, under various activation and transduction conditions.

FIG. 4. In vivo antitumor experiments of CAR-T cells.

FIG. 5. Survival of human T cells in mouse peripheral blood.

FIG. 6. Effects of different concentrations of T cell activators on lentiviral transduction efficiency.

### MODES FOR CARRYING OUT THE INVENTION

After extensive and in-depth research, the inventor unexpectedly found that shortening the activation and transduction time in the preparation of T cells not only does not affect the expression of a recombinant nucleic acid, but improves the proliferation ability and survival time of T cells in vivo. Based on the above findings, the present invention has been completed.

### Terms

The term “cell” and other grammatical forms thereof can refer to cells of human or non-human animal origin.

The term “immune effector cell” refers to a cell which is involved in an immune response and produces immune effects, such as T cells, B cells, natural killer (NK) cells, natural killer T (NKT) cells, dendritic cells, CIK cells, macrophages, mast cells, etc. In some embodiments, the immune effector cell is a T cell, NK cell, NKT cell. In some embodiments, the T cell can be an autologous T cell, xenogeneic T cell, allogeneic T cell. In some embodiments, the NK cell can be an allogeneic NK cell.

The term “artificially engineered cells with immune effector cell function” means that a cell or cell line without immune effects acquires immune effector cell function after being artificially engineered or stimulated by a stimulus. For example, 293T cells are artificially modified to have the function of immune effector cells; and for another example, stem cells are induced in vitro to differentiate into immune effector cells.

In some instances, “T cells” may be pluripotent stem cells from the bone marrow that differentiate and mature into immunocompetent mature T cells within the thymus. In some cases, “T cells” can be a population of cells with specific phenotypic characteristics, or a mixed population of cells with different phenotypic characteristics. For example, “T cells” can be cells comprising at least one subset of following T cells: memory stem cell-like memory T cells (Tscm cells), central memory T cells (Tcm), effector T cells (Tef, Teff), regulatory T cells (tregs) and/or effector memory T cells (Tem). In some instances, “T cells” may be a particular subtype of T cells, such as γδ T cells.

T cells can be obtained from many sources, including PBMC, bone marrow, lymph node tissue, umbilical cord blood, thymus tissue, and tissue from sites of infection, ascites, pleural effusion, spleen tissue, and tumors. In some instances, T cells can be obtained from blood collected from an individual using any techniques known to a skilled person, such as Ficoll™ separation. In one embodiment, cells from the circulating blood of an individual are obtained by apheresis. Apheresis products typically contain lymphocytes, including T cells, monocytes, granulocytes, B cells, other nucleated leukocytes, red blood cells, and platelets. In one embodiment, cells collected by apheresis can be washed to remove plasma molecules and placed in a suitable buffer or medium for subsequent treating steps. Alternatively, cells can be derived from a healthy donor, from a patient diagnosed with a cancer.

The term “chimeric antigen receptor” (CAR) includes an extracellular antigen binding domain, a transmembrane domain, and an intracellular signaling domain. The intracellular signaling domain includes a functional signaling domain of a stimulatory molecule and/or costimulatory molecule. In one aspect, the stimulatory molecule is a (chain bound to a T cell receptor complex; and in another aspect, the cytoplasmic signaling domain further comprises a functional signaling domain of one or more costimulatory molecules, e.g., 4-1BB (i.e., CD137), CD27 and/or CD28.

The term “T cell receptor (TCR)” mediates the recognition of specific major histocompatibility complex (MHC)-restricted peptide antigens by a T cell, including classical TCR receptors and optimized TCR receptors. The classic TCR receptor is composed of two peptide chains, α and β chains. Each peptide chain can be divided into variable region (V region), constant region (C region), transmembrane region and cytoplasmic region. TCR's antigenic specificity depends on the V region, and the V region (Vα, Vβ) has three hypervariable regions, CDR1, CDR2, and CDR3. In one aspect, the specificity of T cells expressing the classical TCR for the target antigen can be induced by a method, such as antigen stimulation of the T cells.

The term “chimeric T cell receptor” includes recombinant polypeptides derived from various polypeptides composed of the TCR, which are capable of binding to surface antigens on target cells, interacting with other polypeptides of the complete TCR complex, and usually located on the T cell surface. The chimeric T cell receptor is composed of a TCR subunit and an antigen binding domain composed of a human or humanized antibody domain, wherein the TCR subunit includes at least part of a TCR extracellular domain, transmembrane domain, and stimulating domain of the intracellular signaling domain of the TCR intracellular domain. The TCR subunit is effectively linked to the antibody domain, wherein the extracellular, transmembrane, and intracellular signaling domains of the TCR subunit are derived from CD3ε or CD3γ, and the chimeric T cell receptor is integrated into the TCR expressed on T cells.

The term “T cell antigen coupler (TAC)” includes three functional domains: 1. antigen binding domain, including single chain antibody, designed ankyrin repeat protein (DARPin) or other targeting groups; 2. extracellular domain, a single-chain antibody that binds to CD3, thereby making the TAC receptor close to the TCR receptor; 3. the transmembrane region and the intracellular region of CD4 co-receptor, wherein the intracellular region is linked to a protein kinase LCK, thereby catalyzing the phosphorylation of immunoreceptor tyrosine activation motifs (ITAM) of the TCR complex as an initial step in T cell activation.

The term “transduction” refers to the introduction of exogenous nucleic acid into a eukaryotic cell.

The term “individual” refers to any animal, such as a mammal or a marsupial. The individual of the present invention includes, but not limited to, a human, non-human primate (e.g., rhesus monkeys or other types of rhesus monkeys), mouse, pig, horse, donkey, cattle, sheep, rat, and poultry of any kind.

The term “peripheral blood mononuclear cell” (PBMC) refers to a cell with a single nucleus in peripheral blood, including a lymphocyte, monocyte, and the like.

The term “T cell activation” or “activation of T cell” refers to the state of T cells that are sufficiently stimulated to induce detectable cell proliferation, cytokine production, and/or detectable effector function.

The term “exogenous” means that a nucleic acid molecule or polypeptide, cell, tissue, and the like, is not endogenously expressed in the organism itself, or the expression level thereof is insufficient to achieve the function that it has when overexpressed.

The term “vector” refers to a nucleic acid molecule which is capable of propagating another nucleic acid molecule to which it is linked. The term includes vectors that are self-replicating nucleic acid structures as well as vectors that are incorporated into the genome of the host cell into which they have been introduced. Certain vectors are capable of directing the expression of nucleic acids to which they are operably linked. Such vectors are referred to herein as “expression vectors” Vectors include viral vectors, such as retroviral vectors, such as lentiviral or gamma retroviral vectors, which have a genome that carries another nucleic acid and is capable of being inserted into the host genome for propagation thereof.

The term “treating” refers to the complete or partial alleviation or reduction of a disease, or a symptom, adverse effects or outcome, or phenotype associated therewith. In certain embodiments, the effects are therapeutic effects, so that the disease or adverse symptoms attributable to it can be partially or completely cured.

“Therapeutically effective amount” refers to an amount effective to achieve desired therapeutic results (e.g., for treating a disease, disorder) and/or pharmacokinetic or pharmacodynamic effects for the treatment at a necessary dosage and period. The therapeutically effective amount may vary depending on factors, such as the disease state, the age, sex and weight of the subject, and the cell population being administered.

The term “multiplicity of infection (MOI)” refers to the ratio of the number of cells infected with virus to the total number of cells in a system.

The term “major histocompatibility complex” (MHC) refers to a protein, usually a glycoprotein, that contains a polymorphic peptide binding site or binding groove. In some cases, the protein can complexe with peptide antigens of polypeptides (including peptide antigens processed by cellular machinery). In some cases, MHC molecules can be displayed or expressed on the cell surface, including as complexes with peptides, i.e., MHC-peptide complexes, for presenting antigen with a conformation which can be recognized by an antigen receptors on T cells (e.g., TCR or TCR-like antibodies). Typically, MHC class I molecules are heterodimers with a membrane spanning a chain, which, in some cases, have three a domains, and non-covalently associated β2 microglobulin. Typically, MHC class II molecules are composed of two transmembrane glycoproteins, a and R, both of which normally span the membrane. The MHC molecule may include the effective portion of the MHC, which contains an antigen binding site or a site for binding peptides and a sequence required for recognition by an appropriate antigen receptor. In some embodiments, MHC class I molecules deliver peptides derived from cytosol to the cell surface, wherein MHC-peptide complexes are identified by T cells (e.g., usually CD8+ T cells, however in some cases CD4+ T cells). In some embodiments, MHC class II molecules deliver peptides derived from the vesicle system to the cell surface, wherein the peptides are normally recognized by CD4+ T cells. Typically, MHC molecules are encoded by a set of linked loci, which are collectively named as H-2 in mice and human leukocyte antigens (HLA) in humans. Therefore, human MHC can also be referred to as human leukocyte antigen (HLA).

The term “MHC-peptide complex” or “peptide-MHC complex” or variants thereof refers to a complex or associate of a peptide antigen with an MHC molecule typically formed, for example, through the non-covalent interactions of the peptide in the binding groove or cleft of the MHC molecule. In some embodiments, the MHC-peptide complex is present or displayed on the cell surface. In some embodiments, the MHC-peptide complex can be specifically recognized by an antigen receptor (e.g., TCR, TCR-like CAR, or antigen-binding portion thereof).

In some embodiments, a peptide of a polypeptide (e.g., a peptide antigen or epitope) can be associated with an MHC molecule for, e.g., recognition by an antigen receptor. Typically, the peptides are derived from or based on fragments of longer biomolecules such as polypeptides or proteins. In some embodiments, the peptides are generally about 8 to about 24 amino acids in length. In some embodiments, the peptides are 9 to 22 amino acids in length for the recognition in MHC class II complexes. In some embodiments, the peptides are 8 to 13 amino acids in length for the recognition in MHC class I complexes. In some embodiments, upon the recognition of a peptide in the context of an MHC molecule (e.g., an MHC-peptide complex), an antigen receptor (e.g., a TCR or TCR-like CAR) generates or triggers an activation signal to T cells, thereby inducing a T cell response, such as T cell proliferation, cytokine production, cytotoxic T cell responses or other responses.

The present invention provides methods for transducing viral vectors into cells (e.g., immune effector cells) involving activation and transduction of cells to be transduced. The activation and transduction of cells may be performed simultaneously, i.e. co-incubating an input composition comprising cells to be transduced, a stimulator of the cells to be transduced, and viral vector particles carrying a recombinant nucleic acid. Or the activation of cells may be performed and then transduced. For example, the input composition comprising the cells to be transduced and the stimulator of the cells to be transduced are co-incubate for activation, and then viral vector particles carrying the recombinant nucleic acid are added for incubation. The total time of the transduction and activation of the recombinant nucleic acid and transduction is controlled within 72 hours, preferably within 48 hours, or within 36 hours, or within 24 hours.

In some embodiments, the provided methods involve incubating and/or contacting retroviral vector particles (e.g., lentiviral vectors) with a population of cells (e.g., immune cells, such as, T cells). Before and/or concurrently with and/or after contacting or incubating the cells with viral particles, T cells are activated using ex vivo cell activation reagents (e.g., anti-CD3/anti-CD28 reagents). Preferably, cells are activated prior to viral transduction.

In a specific embodiment, when the input composition comprising the cells to be transduced, the stimulator of the cells to be transduced, and the viral vector particles carrying the recombinant nucleic acid are co-incubated, the incubation time does not exceed 72 hours for harvesting to obtain an output composition containing cells transduced with the recombinant nucleic acid. Preferably, the incubation time can be 1-72 hours; more preferably, the incubation time can be 2-48 hours; more preferably, the incubation time can be 2-36 hours; more preferably, the incubation time can be 12-36 hours; more preferably, the incubation time can be 12-24 hours; and more preferably, the incubation time can be 15-24 hours. In a specific embodiment, after the output composition is purified by washing, centrifugation, etc., a pharmaceutical preparation can be prepared without further in vitro expansion and culture, that is, the drug prepared by using the output composition is used to a subject (or patient) without in vitro expansion.

In a specific embodiment, the method for transducing cells with a viral vector comprises following steps: step (1), incubating an input composition comprising a cell to be transduced and a stimulator of the cell to be transduced for not more than 72 h, step (2)), then adding viral vector particles of a recombinant nucleic acid and incubating for not more than 24 hours, step (3), harvesting to obtain an output composition comprising cells transduced with recombinant nucleic acid. Preferably, the total incubation time of (1) and (2) is not more than 72 hours; more preferably, the total incubation time of (1) and (2) is not more than 60 h, or not more than 48 h, or not more than 32 h, or not more than 28 h; and more preferably, the total incubation time of (1) and (2) is not more than 24 h. In a specific embodiment, the incubation time of step (1) is 2-72 hours; preferably, the incubation time of step (1) is 2-71 hours; more preferably, the incubation time of step (1) is 2-48 hours; more preferably, the incubation time of step (1) is 2-32 hours; more preferably, the incubation time of step (1) is 2-28 hours; more preferably, the incubation time of step (1) is 3-24 hours; more preferably, the incubation time of step (1) is 5-24 hours; more preferably, the incubation time of step (1) is 7-24 hours; more preferably, the incubation time of step (1) is 7-23 hours; more preferably, the incubation time of step (1) is 10-23 hours; more preferably, the incubation time of step (1) is 15-23 hours; and more preferably, the incubation time of step (1) is 15-22 hours. In a specific embodiment, the incubation time of step (2) is 30 mins-24 hours; preferably, the incubation time of step (2) is 30 mins-21 hours; preferably, the incubation time of step (2) is 30 mins-17 hours; preferably, the incubation time of step (2) is 30 mins-12 hours; preferably, the incubation time of step (2) is 30 mins-10 hours; preferably, the incubation time of step (2) is 30 mins-8 hours; preferably, the incubation time of step (2) is 1-8 hours; preferably, the incubation time of step (2) is 1-4 hours; and more preferably, the incubation time of step (2) is 1-3 hours.

In some embodiments, the recombinant nucleic acid may encode a receptor that recognizes a specific target antigen, such as a T cell receptor (TCR), a chimeric antigen receptor (CAR), a chimeric T cell receptor, or a T cell antigen coupled controller (TAC).

In some embodiments, the specific target antigen is a disease-associated antigen or a universal tag.

In some embodiments, the disease is cancer, an autoimmune disease, or an infectious disease.

In some embodiments, the specific target antigen is a tumor-associated antigen, such as: B cell maturation antigen (BCMA), carbonic anhydrase 9 (CAIX), tEGFR, Her2/neu (receptor tyrosine kinase erbB2), CD19, CD20, CD22, Mesothelin, CEA, CD23, CD24, CD30, CD33, CD38, CD44, EGFR, Epiglin 2 (EPG-2), Epiglin 40 (EPG-40), EPHa2, erb-B2, erb-B3, erb-B4, erbB dimer, EGFR vIII, folate binding protein (FBP), FCRL5, FCRH5, fetal acetylcholine receptor, GD2, GD3, HMW-MAA, IL-22R-α, IL-13R-α2, Kinase Insertion Domain Receptor (kdr), L1 Cell Adhesion Molecule (L1-CAM), Melanoma-Associated Antigen (MAGE), TAG72, B7-H6, IL-13 Receptor α2 (IL-13Ra2), CA9, GD3, HMW-MAA, CD171, G250/CAIX, HLA-AI MAGEA1, HLA-A2, PSCA, folate receptor, CD44v6, CD44v7/8, avb6 integrin, 8H9, NCAM, VEGF receptor, 5T4, Fetal AchR, NKG2D ligand, CD44v6, mesothelin, mucin 1 (MUC1), MUC16, PSCA, NKG2D, NY-ESO-1, MART-1, gp100, carcinoembryonic antigen, G protein-coupled receptor 5D (GPCR5D), ROR1, TAG72, VEGF-R2, Carcinoembryonic Antigen (CEA), Prostate Specific Antigen, PSMA, Ephrin B2, CD123, c-Met, GD-2, O-acetylated GD2 (OGD2), CE7, Wilms tumor 1 (WT-1), cyclin, CCL-1, CD138, Claudin18.2, GPC3.

Resulting cells transduced with a recombinant nucleic acid can be used for adoptive immunotherapy. In such embodiments, the provided methods can be used to prepare immune cells, such as T cells, for adoptive therapy. The total time of activation and transduction of the method is controlled within 24 hours, or 36 hours, or 48 hours, or 72 hours. In some aspects, the provided methods reduce the time to engineer and/or prepare cells for adoptive cell therapy.

In some embodiments, the input composition comprises a primary cell population that has been obtained from a subject's sample and/or enriched for a particular subset of cells (e.g., T cells). In some embodiments, the population of cells (e.g., the input composition) may be a population of cells that has previously been cryopreserved. In some embodiments, the incubation and/or exposure was initiated at no more than or no more than about 1 hour, 3 hours, 6 hours, 12 hours, 18 hours, 24 hours, at 48 hours or 72 hours after the primary cell-containing sample (e.g., an apheresis sample) is obtained from the subject. In some embodiments, the method produces an output composition wherein at least 25%, at least 30%, at least 40%, at least 50%, or at least 75% of the total cells (or specific target cell types, e.g., T cells) in the output composition are transduced with the viral vector and/or express the recombinant gene product encoded thereby. In some embodiments, at least 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% or 95% of cells (e.g., T cells) in the cell population (e.g., the output composition) are transduced with retroviral vector particles according to the provided methods.

Methods for assessing the expression of T cell activation markers are known in the art. Antibodies and reagents for detecting such labels are well known in the art and readily available. Assays and methods for detecting such markers include, but not limited to, flow cytometry (including intracellular flow cytometry), ELISA, ELISPOT, cytometric bead arrays or other multiplex methods, Western blotting, and other immunoaffinity-based methods. In some embodiments, the methods are capable of achieving at least a specific transduction efficiency under certain conditions.

In some embodiments, the provided methods can further comprise a cryopreservation step before or after incubating (e.g., transducing) the cells with the viral particles. In some embodiments, this step may provide preservation of the cell product, such as cell preservation in transit, or cell preservation after preparation.

In some embodiments, the activation or stimulation can be performed ex vivo or in vivo. In some embodiments, after being incubated (e.g., transduced) with viral particles, the cells can be infused into a patient for in vivo activation and expansion.

In some embodiments, the used stimulator for the cells to be transduced can be one stimulator, two stimulators, or a combination of several stimulator. For example, a T cell activator can be a CD3 binding molecule (such as CD3 antibody), CD28 binding molecule (such as CD28 antibody), recombinant IL-2, recombinant IL-15, recombinant IL-7, recombinant IL-21, or a mixture of at least two species, such as antibodies to CD3 and antibodies to CD28, or antibodies to CD3, antibodies to CD28, or IL2.

In some embodiments, the multiplicity of infection of the viral vector particles is not higher than 20; preferably, the multiplicity of infection is 0.5-20; more preferably, the multiplicity of infection is 1.5-20; more preferably, the multiplicity of infection is is 3-20; and more preferably, the multiplicity of infection is 3-12.

In some embodiments, during or after the incubation, the provided methods can further comprise culturing the input composition, output composition, and/or transduced cells ex vivo, for example, under conditions that activate the cells, to induce proliferation and/or activation thereof. The activation is carried out in the presence of one or more activators. In some embodiments, the activator can be a CD3 binding molecule, a CD28 binding molecule, or a cytokine (e.g., recombinant IL-2, recombinant IL-15, recombinant IL-7, or recombinant IL-21). In some embodiments, the binding molecule is an antibody or antigen-binding fragment, e.g., an anti-CD3 antibody and/or an anti-CD28 antibody. In some embodiments, the further culturing is performed under conditions that achieve cell expansion, to produce a therapeutically effective dose of the cells for the administration to a subject by adoptive cell therapy.

In some embodiments, the provided methods avoid and/or minimize significant changes in the differentiation state of T cells ex vivo during introduction, transfer, and/or transduction of T cells with a nucleic acid encoding a recombinant receptor (e.g., CAR). In some embodiments, memory T cells are generated according to the provided methods, including stem cell memory T cells, central memory T cells, effector memory T cells.

In some embodiments, the proportion of cells transduced with the recombinant nucleic acid (e.g., CAR T cells) contained in the output composition of the cells of the present invention is lower than that achieved by conventional processes. In a specific embodiment, the number of cells is not higher than 1*10¹⁰, 1*10⁹, 1*10⁸, 1*10⁷, 1*10⁶, 1*10⁵, or 1*10⁴.

The content of cells with a memory cell phenotype (e.g., memory T cells) in the output composition of the present invention comprising cells transduced with the recombinant nucleic acid is higher than that achieved by conventional processes. In some embodiments, the content is at least 1.5 times, 2 times, 3 times, 4 times, or 5 times higher.

In some embodiments, the memory T cells are cells with a T central cell memory (TCM) phenotype, e.g., CD45RO+CCR7+CD62L+ T cells and/or CD45RO+CCR7+CD27+CD28+CD62L+ T cells.

In some embodiments, one, more or all of the steps in the preparation of cells of the invention for clinical uses (e.g., in adoptive cell therapy) are performed under sterile conditions. In some embodiments, one or more processes by which cells are enriched, activated, transduced, or washed are performed in a closed system.

In some embodiments, the cells are treated ex vivo for a shorter period of time, for further shortening the period.

In some embodiments, the cells transduced with a recombinant nucleic acid (e.g., CAR T cells) produced by the provided methods exhibit longer persistence and/or reduced cell depletion when administered to a subject.

In some embodiments, the cells transduced with a recombinant nucleic acid (e.g., CAR T cells) produced by the provided methods exhibit better efficacy when administered to a subject.

In some embodiments, the provided methods reduce the variability of cells during the manufacture of cell therapy products.

In some embodiments, eliminating the time for ex vivo activation and transduction of cells improves the process of preparing cells transduced with a recombinant nucleic acid for adoptive immunotherapy.

### Activation and Transduction Methods

Provided herein is a method for incubating or contacting an input composition (including cells to be transduced) with retroviral vector particles (e.g., lentiviral vector particles). In some aspects, the input composition is a composition of primary cells obtained from a subject, wherein, in some cases, a subpopulation or subset of cells has been selected and/or enriched. For example, when the cells to be transduced are T cells, the input composition can be a T cell population, an enriched T cell population, or PBMC.

In some embodiments, the cells comprise one or more nucleic acids introduced by genetic engineering according to the provided methods, thereby expressing recombinant or genetically engineered products of such nucleic acids. In some embodiments, the nucleic acid is heterologous, i.e., not normally present in a cell or a sample obtained from a cell, such as a nucleic acid obtained from another organism or cell. For example, the nucleic acid is typically not found in the engineered cells and/or the organism from which such cells are derived. In some embodiments, the nucleic acid is not a naturally occurring nucleic acid, such as a nucleic acid not found in nature, including nucleic acids encoding chimeric combinations of nucleic acids from various domains of a variety of different cell types.

Treating steps of the method may comprise any one or more of a plurality of cell treating steps, alone or in combination. In certain embodiments, the treating steps comprises transducing the cells with viral vector particles containing a retroviral vector, e.g., a vector encoding a recombinant product for the expression in the cell. The method may further and/or alternatively include other treating steps, such as steps of isolation, isolation, selection, washing, suspension, dilution, concentration and/or formulation of cells. In some cases, the method may further include an ex vivo culturing step (e.g., activating the cells to induce the proliferation and/or activation thereof). In other cases, the step of activating the cells is performed in vivo after the cells are administered to the subject, performed by antigen recognition and/or performed after one or more agents are administered to enhance or expand the expansion, activation and/or proliferation of the cells in the subject. In some embodiments, the method comprises isolating cells from a subject, and preparing, treating, culturing and/or engineering them, and reintroducing them into the same subject before or after cryopreservation.

In some embodiments, the method comprises treating steps performed in the following order, wherein: primary cells are first separated (e.g., selected or isolated) from a biological sample; the selected cells are activated, amplified or multiplied ex vivo in the presence of an activation agent, and then viral vector particles are added for incubation and transduction, the total time of activation and transduction is not more than or not more than about 24 or 36 or 48 hours, wherein the time of transduction is at least or at least about 1, 2, 3, 4, 5, 6, 7, 8, 9, 10, 11, 12, 13, 14, 15, 16, 17, 18, 19, 20, 21, 22, 23, 24 hours.

In some cases, the transduced cells are activated, expanded or propagated ex vivo, for example, in the presence of an activating agent. In some embodiments, the method can include one or more treating steps selected from washing, suspending, diluting, and/or concentrating cells, which can be performed before, during, simultaneously with or after the separation (e.g., isolation or selection), activation, transduction, and/or formulation step.

In some embodiments, one or more or all of the treating steps (e.g., isolation, selection and/or enrichment, treatment, activation, incubation combined with transduction and engineering) and formulation steps are performed using an integrated or self-contained system, device or equipment and/or in an automated or programmable manner.

In some embodiments, one or more cell treating steps in conjunction with preparing, treating and/or incubating cells in conjunction with the provided transduction methods can be performed in culture bags or flasks, which can provide certain advantages as compared with other available methods.

In some embodiments, the system comprises a series of containers, such as bags, tubing, stopcocks, clips, connectors, and centrifuge chambers. In some embodiments, a container (e.g., culture bag or flask) includes one or more containers (e.g., culture bag or flask) in the same container or separate containers (e.g., the same culture bag or flask; or separate culture bags or flasks) containing the cells to be transduced and viral vector particles.

In some embodiments, the system (e.g., a closed system) is sterile.

In some embodiments, the system may be disposable, such as a disposable culture bag or culture bottle.

### A. Preparation of Sample and Cell

The cells are usually eukaryotic cells, such as mammalian cells, and usually human cells. In some embodiments, the cells are derived from blood, bone marrow, lymphoid, or lymphoid organs, and are cells of the immune system, such as innate or adaptive immune cells, such as bone marrow or lymphocytes, including lymphocytes, typically T cells and and/or NK cells. Other exemplary cells include stem cells, such as pluripotent stem cells and pluripotent stem cells, including induced pluripotent stem cells (iPSCs).

The cells are typically primary cells, such as those isolated directly from a subject and/or isolated from a subject and frozen. In some embodiments, the cells include one or more subsets of T cells or other cell types, such as the entire population of T cells, CD4+ cells, CD8+ cells, and subsets thereof, such as those subsets defined by: function, activation status, maturity, potential for differentiation, expansion, recycling, localization and/or persistence capacity, antigen specificity, antigen receptor type, presence in specific organs or compartments, markers or cytokine secretion characteristics and/or degree of differentiation. With respect to the subject to be treated, the cells may be allogeneic and/or autologous. The methods include available methods. In some aspects, as for the prior art, the cells are pluripotent and/or multipotent, such as stem cells, such as induced pluripotent stem cells (iPSCs). In some embodiments, the method comprises isolating cells from a subject, preparing, treating, culturing and/or engineering them, and reintroducing them into the same subject before or after cryopreservation.

Among the subtypes and subpopulations of T cells and/or CD4+ and/or CD8+ T cells, there are naive T (TN) cells (or Naive T cells), effector T cells (TEFF), memory T cells and subtypes thereof (e.g., stem cell memory T (TSCM), central memory T (TCM), effector memory T (TEM) or terminally differentiated effector memory T cells), tumor infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells (such as TH1 cells, TH2 cells, TH3 cells, TH17 cells, TH9 cells cells, TH22 cells, follicular helper T cells), α/β T cells and δ/γ T cells.

In some embodiments, the cells are natural killer (NK) cells. In some embodiments, the cells are monocytes or granulocytes, such as myeloid cells, macrophages, neutrophils, dendritic cells, mast cells, eosinophils and/or basophils.

In some embodiments, the cells are derived from a cell line, e.g., a T cell line. In some embodiments, the cells are obtained from xenogeneic sources, e.g., from mice, rats, non-human primates, and pigs.

In some embodiments, cells can be isolated from a sample, e.g., a biological sample, such as a sample obtained from or derived from a subject. In some embodiments, the subject from which the cells are isolated is a subject suffering from a disease or in need of cell therapy or to which cell therapy is to be administered. In some embodiments, the subject is a human in need of specific therapeutic intervention (e.g., adoptive cell therapy, in which cells are isolated, processed, and/or engineered).

In some embodiments, the cells are primary cells, e.g., primary human cells. The sample includes a tissue, fluid, and other sample taken directly from a subject, as well as a sample from one or more processing steps (e.g., isolation, centrifugation, genetic engineering (e.g., transduction with a viral vector), washing, and/or incubation). The biological sample may be a sample obtained directly from a biological source or a processed sample. The biological sample includes, but not limited to, body fluid (e.g., blood, plasma, serum, cerebrospinal fluid, synovial fluid, urine, and sweat), tissue and an organ sample, including processed samples derived therefrom.

In some aspects, the sample from which cells are derived or isolated is a blood or a blood-derived sample, or is or is derived from an apheresis or leukapheresis product. Exemplary samples include whole blood, peripheral blood mononuclear cells (PBMC), leukocytes, bone marrow, thymus, tissue biopsy, tumor, leukemia, lymphoma, lymph node, gut-associated lymphoid tissue, mucosa-associated lymphoid tissue, spleen, other lymphoid tissue, liver, lung, stomach, intestine, colon, kidney, pancreas, breast, bone, prostate, cervix, testis, ovary, tonsil or other organs and/or cells derived therefrom. In the context of cell therapy (e.g., adoptive cell therapy), the sample includes a sample from autologous and allogeneic sources.

In some instances, cells from the subject's circulating blood are obtained, e.g., by apheresis or leukapheresis. In some aspects, the sample contains lymphocytes, including T cells, monocytes, granulocytes, B cells, other nucleated leukocytes, red blood cells, and/or platelets, and in some aspects, cells other than red blood cells and platelets.

In some embodiments, a blood sample collected from a subject is washed, e.g., to remove plasma fractions and cells are placed in an appropriate buffer or medium for subsequent processing steps. In some embodiments, the cells are washed with phosphate buffered saline (PBS). In some embodiments, the wash solution is deficient in calcium and/or magnesium and/or many or all divalent cations. In some aspects, the washing step is accomplished by an automatic or semi-automatic “flow-through” centrifuge (e.g., Cobe2991 Cell Processor, Baxter, MACS PLUS) according to the manufacturer's instructions. In some aspects, the washing step is accomplished by tangential flow filtration (TFF) according to the manufacturer's instructions. In some embodiments, the cells are resuspended in various biocompatible buffers (e.g., PBS without Ca++/Mg++) after washing. In certain embodiments, components of the blood cell sample are removed and the cells are directly resuspended in culture medium.

In some embodiments, the sample is contacted with and/or contains serum or plasma (e.g., human serum or plasma) prior to enrichment and/or selection of cells. In some embodiments, the serum or plasma is autologous to the subject from which the cells are obtained. In some embodiments, the serum or plasma is present in the sample at following concentrations: at least or at least about 10% (v/v), at least or at least about 15% (v/v), at least or at least about 20% (v/v) v), at least or at least about 25% (v/v), at least or at least about 30% (v/v), at least or at least about 35% (v/v) or at least or at least about 40% (v/v). In some embodiments, the sample containing primary cells is contacted with or contains an anticoagulant prior to the selection and/or transduction of cells. In some embodiments, the anticoagulant is or contains free citrate ions, e.g., an anticoagulant citrate dextrose solution, solution A (ACD-A).

In some embodiments, cells from the sample are transferred or suspended in a serum-free medium prior to the enrichment and/or selection of cells. In some embodiments, the serum-free medium is a defined and/or well-defined cell culture medium. In some embodiments, the serum-free medium is formulated to support the growth, proliferation, health, homeostasis of cells of a certain cell type (e.g., immune cells, T cells, and/or CD4+ and CD8+ T cells).

In some embodiments, the sample is maintained or kept at a temperature of 2° C. to 8° C. for up to 48 hours, e.g., up to 12 hours, 24 hours, or 36 hours.

In some embodiments, the preparation method includes the step of freezing (e.g., cryopreserving) the cells before or after isolation, selection and/or enrichment and/or incubation for transduction and engineering. In some embodiments, granulocytes in the cell population can be removed by the freezing and subsequent thawing steps, and monocytes can be, to some extent, removed. In some embodiments, the cells are suspended in a freezing solution to remove plasma and platelets, for example, after the washing step. In some aspects, a variety of known freezing solutions can be used. In some embodiments, the T cells are cryopreserved in the presence of a cryoprotectant. An example involves the use of PBS containing 20% DMSO and 8% human serum albumin (HSA), or other suitable cell freezing medium. Cells are then typically frozen to −80° C. and stored in the gas phase of a liquid nitrogen storage tank.

In some embodiments, the isolation of cells includes one or more preparative and/or non-affinity-based cell isolation steps. In some instances, the cells are washed, centrifuged, and/or incubated in the presence of one or more reagents, for example, to remove unwanted components, enrich for desired components, lyse or remove cells sensitive to a particular reagent. In some examples, the cells are isolated based on one or more properties (e.g., density, adhesion properties, size, sensitivity and/or resistance to particular components).

In some embodiments, the isolation method comprises separating different cell types based on the expression or presence of one or more specific molecules (e.g., surface markers, such as surface proteins, intracellular markers, or nucleic acids) in the cells. In some embodiments, any known methods for the isolation based on such markers can be used. In some embodiments, the isolation is an affinity or immunoaffinity-based isolation. For example, in some aspects, the isolation comprises isolating cells and cell populations based on the expression or expression level of one or more markers (usually cell surface markers) of the cells. For example, cells are incubated with antibodies or binding partners that specifically bind to such labels, followed by typically a washing step, so that the cells bound to the antibodies or binding partners can be separated from those cells not bound to the antibodies or binding partners partner.

Such isolation step may be based on positive selection (wherein cells that have bound the agent are retained for further use) and/or negative selection (wherein cells not bound to the antibody or binding partner are retained). It is not necessary to 100% enrich or remove specific cell populations or cells expressing specific markers by the isolation. For example, the positive selection or enrichment for a particular type of cells (such as those expressing a marker) means that the number or percentage of such cells is increased, but it is not necessary to result in the complete absence of cells that do not express the marker. Similarly, the negative selection, removal, or depletion of a particular type of cells (such as those expressing a marker) means that the number or percentage of such cells is reduced, but it is not necessary to result in the complete removal of such cells.

For example, in some aspects, a specific subset of T cells, such as cells that are positive or express high levels of one or more surface markers (e.g., CD28+, CD62L+, CCR7+, CD27+, CD127+, CD4+, CD8+, CD45RA+ and/or CD45RO+ T cells) were isolated by positive or negative selection techniques. CD3+, CD28+ T cells can be positively selected using anti-CD3/anti-CD28-conjugated magnetic beads or microbeads (e.g., M-450CD3/CD28T Cell Expander).

In some embodiments, the isolation is performed by enrichment for a particular cell population by positive selection or depletion for a particular cell population by negative selection. In some embodiments, the positive or negative selection is accomplished by incubating cells with one or more antibodies or other binding agents that interact with the cells, respectively. The one or more antibodies or other binding agents specifically bind to one or more surface markers expressed, or expressed at relatively high levels (high label) (label+) on the positively or negatively selected cells, respectively.

In some embodiments, T cells are isolated from the PBMC sample by negative selection for markers expressed on non-T cells (e.g., B cells, monocytes, or other leukocytes, such as CD14). In some aspects, the CD4+ or CD8+ selection step is used to isolate CD4+ helper T cells and CD8+ cytotoxic T cells. Such CD4+ and CD8+ populations can be further classified into subpopulations by positive or negative selection for markers expressed on or expressed at a relatively high degree on one or more naive, memory and/or effector T cell subsets.

In some embodiments, CD8+ cells are further enriched or depleted for naive, central memory, effector memory and/or central memory stem cells by, for example, positive or negative selection based on surface antigens associated with the corresponding subpopulation. In some embodiments, central memory T (TCM) cells are enriched to increase efficacy to, for example, improve long-term survival, expansion and/or engraftment following administration thereof. In some embodiments, the combination of TCM-enriched CD8+ T cells and CD4+ T cells further enhance the efficacy.

In some embodiments, memory T cells are present in both of CD62L+ and CD62L-subsets of CD8+ peripheral blood lymphocytes. PBMCs can be enriched or depleted for CD62L-CD8+ and/or CD62L+CD8+ fractions by, for example, using anti-CD8 and anti-CD62L antibodies.

In some embodiments, the enrichment of central memory T (TCM) cells is based on positive or high surface expression of CD45RO, CD62L, CCR7, CD28, CD3 and/or CD127. In some aspects, it is based on negative selection of cells expressing or highly expressing CD45RA and/or granzyme B. In some aspects, the TCM cell-enriched CD8+ population is isolated by depletion of cells expressing CD4, CD14, CD45RA and positive selection or enrichment of cells expressing CD62L. In one aspect, starting from a negative cell fraction selected based on CD4 expression, central memory T (TCM) cells are enriched, and the negative cell fraction is negatively selected based on the expression of CD14 and CD45RA and positively selected according to CD62L.

In certain instances, PBMC samples or other leukocyte samples are subjected to selection for CD4+ cells, in which negative and positive fractions are retained. The negative fractions are then negatively selected based on the expression of CD14 and CD45RA or CD19, and positively selected based on marker characteristics of central memory T cells, such as CD62L or CCR7, wherein the positive and negative selections are performed in any order.

CD4+T helper cells are classified into naive, central memory and effector cells by identifying cell populations with cell surface antigens. CD4+ lymphocytes can be obtained by standard methods. In some embodiments, the naive CD4+T lymphocytes are CD45RO−, CD45RA+, CD62L+, CD4+ T cells. In some embodiments, the central memory CD4+ cells are CD62L+ and CD45RO+. In some embodiments, the effector CD4+ cells are CD62L− and CD45RO−.

In one example, for enriching CD4+ cells by negative selection, a monoclonal antibody cocktail typically includes antibodies against CD14, CD20, CD11b, CD16, HLA-DR, and CD8. In some embodiments, the antibody or binding partner is bound to a solid support (e.g., beads) or matrix (e.g., magnetic or paramagnetic beads or microbeads) to allow cell isolation for positive and/or negative selection. For example, in some embodiments, immunomagnetic (or affinity magnetic) separation techniques are used to separate or isolate cells and cell populations.

In some embodiments, the T cell activator is a solid support (e.g., beads, including magnetic beads and/or microbeads; polymeric matrixes, including polymer nanomatrixes) conjugated to anti-CD3 and/or anti-CD28 and/or anti-41-BB monoclonal antibodies.

In some aspects, the sample or composition of cells to be isolated is incubated with small magnetizable or magnetically responsive material (e.g., magnetically responsive particles or microparticles, such as paramagnetic beads (e.g., Dynabeads or MACS beads). In some embodiments, the magnetic particles or beads comprise a magnetically responsive material bound to a specific binding member, such as an antibody or other binding partner. There are many well-known magnetically responsive materials used in magnetic separation methods.

The incubation is typically carried out under conditions whereby the antibody or binding partner, or a molecule (such as a secondary antibody or other reagent) that specifically binds to such an antibody or binding partner attached to magnetic particles or beads, specifically bind to cell surface molecules (if present on cells within the sample).

In some aspects, the sample is placed in a magnetic field, and those cells with magnetically responsive or magnetizable particles attached thereto will be attracted to the magnet and separated from unlabeled cells. For positive selection, cells attracted by the magnet were retained; and for negative selection, cells that were not attracted (unlabeled cells) were retained.

In some embodiments, the magnetically responsive particles or beads remain attached to the cells, which are subsequently incubated, cultured, and/or engineered; and in some aspects, the particles or beads remain attached to the cells for the administration to a patient. In some embodiments, magnetizable or magnetically responsive particles are removed from the cells. Methods for removing magnetizable particles or beads from cells are known and include, for example, the use of competing non-labeled antibodies and magnetizable particles or antibodies or beads conjugated to cleavable linkers. In some embodiments, the magnetizable particles are biodegradable.

In some embodiments, the affinity-based selection is performed via Magnetic Activated Cell Sorting (MACS) (Miltenyi Biotech, Auburn, CA). The Magnetic Activated Cell Sorting (MACS) system enables high-purity selection of cells with attached magnetized particles. In certain embodiments, the MACS operates in a mode in which non-target and target species are sequentially eluted following application of an external magnetic field. In other words, cells attached to the magnetized particles remain in an appropriate place, while unattached species are eluted. Afterwards, after the first elution step is completed, the species, which are trapped in the magnetic field and prevented from being eluted, are released in a way that allows them to be eluted and recovered. In certain embodiments, the non-target cells are labeled and depleted from a heterogeneous population of cells.

In some embodiments, the method comprises selecting cells, wherein all or part of the selection is performed in the lumen of a centrifuge chamber, for example, under centrifugation spinning. In some embodiments, the cells are incubated with a selection reagent (e.g., an immunoaffinity-based selection reagent) in a centrifuge chamber. For example, an immunoaffinity-based selection will depend on the favorable energetic interaction between the isolated cells and a labeled molecule (e.g., an antibody or other binding partner on a solid (e.g., particle)) that specifically binds to the cell. In certain available methods for affinity-based separation using particles (e.g., beads), the particles and cells are incubated in a container (e.g., a tube or bag) and shaken or mixed simultaneously, and the ratio of cell density to particles (e.g., beads) is constant, so that the energetically favorable interaction will be promoted. Such an approach may not be ideal for large-scale production, since, for example, a large volume may be required to maintain optimal or desired cell-to-particle ratios, while maintaining desired cell numbers. Therefore, such approache may need to be processed in a batch mode or format, which may require increased time, number of steps and operations, thereby increasing cost and risk of user error.

In some embodiments, at least a portion of the selection step is performed in a centrifugation chamber, which includes incubating cells with a selection reagent. In some aspects of such processes, a volume of cells is mixed with an amount of the desired affinity-based selection reagent, and said volume and amount are significantly less than those typically used when a similar selection is made in a tube or container to select the same number of cells and/or the same volume of cells according to the manufacturer's instructions. In some embodiments, the employed amount of one or more selection reagents is not more than 105%, not more than 110%, not more than 115%, not more than 120%, not more than 125%, not more than 150%, not more than 160%, not more than 170%, or not more than 180% of the amount of the same one or more selection reagents used to select cells in a tube- or vessel-based incubation for the same number of cells and/or the same volume of cells according to the manufacturer's instructions.

For example, as part of a selection method that can be performed in a chamber, incubating with one or more selection reagents includes the use of one or more selection reagents. One or more different cell types are selected based on the expression or presence of one or more specific molecules (e.g., surface markers, such as surface proteins, intracellular markers, or nucleic acids) in or on cells. In some embodiments, the separation based on such labels can be performed by any known method using one or more selection reagents. In some embodiments, the one or more selection reagents result in separation, which is based on affinity or immunoaffinity. For example, in some aspects, the selection includes incubating with one or more reagents to separate cells and cell populations based on cellular expression or expression levels of one or more markers (usually cell surface markers). For example, the cells or cell populations are incubated with antibodies or binding partners specifically binding to such labels, and then typically subjected to washing steps, so that cells that have bound the antibody or binding partner can be separated from those cells that are not bound to the antibody or binding partner.

In some embodiments, for the selection of cells, e.g., selection based on immunoaffinity, the cells are incubated in a chamber cavity with a composition that also contains a selection buffer with a selection reagent such as a molecule, such as an antibody, that specifically binds to a surface label of the cells desired to be enriched and/or depleted (but not to other cells in the composition), and optionally coupled to a scaffold (such as a polymer or surface, such as beads, such as magnetic beads or microbeads, such as magnetic beads or microbeads coupled to monoclonal antibodies specific for CD4 and CD8). In some embodiments, the total duration of incubation with the selection reagent is 5 mins to 6 hrs, e.g., 30 mins to 3 hrs, e.g., at least 30 mins, 60 mins, 120 mins, or 180 mins. In some embodiments, the incubation is typically performed under a mixed condition, for example, in the presence of rotation, typically at a relatively low force or speed, e.g., lower than the speed used to pellet the cells, e.g., 600 rpm to 1700 rpm (e.g., at least 600 rpm, 1000 rpm or 1500 rpm or 1700 rpm), for example, under a certain RCF at the sample or chamber wall or other vessel wall, which is 80 g to 100 g (e.g., at least 80 g, 85 g, 90 g, 95 g or 100 g).

In some embodiments, following incubation with the selection reagent, the incubated cells (including cells which has bound to the selection reagent) are forced out of the centrifuge chamber, for example, transferred from the centrifuge chamber into a system for the immunoaffinity-based isolation of cells. In some embodiments, the system for immunoaffinity-based isolation is or contains a magnetic isolation column. In some embodiments, one or more other treating steps, such as a washing step, may be performed in the chamber prior to isolation.

In some aspects, CliniMACS system (Miltenyi Biotic) is used for the isolation and/or other steps, e.g., for the automated isolation of cells at a clinical scale level in a closed and sterile system.

In some embodiments, CliniMACS Prodigy system (Miltenyi Biotic) is used for the isolation and/or other steps. In some aspects, the CliniMACS Prodigy system is equipped with a cell processing complex that allows the automated washing and fractionation of cells by centrifugation. In some embodiments, the cell populations described herein are collected and enriched (or depleted) by flow cytometry, wherein cells stained for various cell surface markers are carried in a fluid flow. In some embodiments, the cell populations described herein are collected and enriched (or depleted) by preparative scale (FACS) sorting.

In some embodiments, the antibody or binding partner is labeled with one or more detectable labels to facilitate isolation for positive and/or negative selection. For example, the isolation can be based on the binding to fluorescently labeled antibodies. In some examples, cells which are isolated based on the binding of antibodies or other binding partners specific for one or more cell surface markers are carried in fluid flow, such as by fluorescence-activated cell sorting (FACS), including preparative scale (FACS) and/or Micro Electro Mechanical Systems (MEMS) chips, e.g., in combination with flow cytometry systems. Such methods allow simultaneous positive and negative selection based on multiple markers.

Activation and/or Expansion of Cells Before or Simultaneously with Transduction

In some embodiments, the screened cells (e.g., the input composition) are incubated and/or cultured in conjunction with genetic engineering. The incubation step may include the activation and transduction to allow the integration of the viral vector into the host genome of one or more cells. Incubation and/or engineering can be performed in culture vessels such as cells, chambers, wells, columns, tubes, sets of tubes, valves, vials, dishes, bags, or other containers used to culture or grow cells. In some embodiments, the composition or cell is incubated in the presence of stimulating conditions or activating agents. These conditions include those designed for inducing proliferation, expansion, activation and/or survival of cells in a population, for simulating antigen exposure and/or for priming cells for genetic engineering, such as for the introduction of recombinant antigen receptors.

In some embodiments, a further incubation is performed under conditions for stimulation and/or activation of the cells, and the conditions may include one or more of the following: a particular medium, temperature, oxygen content, carbon dioxide amounts, timing, agents (e.g., nutrients), amino acids, antibiotics, ions and/or stimulatory factors (e.g. cytokines, chemokines), antigens, binding partners, fusion proteins, recombinant soluble receptors and any other agents designed to activate cells.

In some embodiments, the consitions for the activation or agents include one or more agents (e.g., stimulatory and/or auxiliary agents), such as, ligands, capable of activating the intracellular signaling domain of a TCR complex. In some aspects, the agent turns on or initiates TCR/CD3 intracellular signaling cascade in T cells, which is, for example, an agent suitable for delivering primary signals to, for example, initiate activation of ITAM-induced signals (e.g., those specific for TCR components), and/or an agent promoting co-stimulatory signals (e.g, co-stimulatory signals specific for T cell costimulatory receptors), such as anti-CD3, anti-CD28, or anti-41-BB (e.g., which optionally bound to a solid support (e.g., beads)) and/or one or more cytokines. The stimulating agents include anti-CD3/anti-CD28 beads (e.g., DYNABEADS® M-450 CD3/CD28 T cell expansion agent and/or ExpACT® beads). Optionally, the activation method may further comprise a step of adding anti-CD3 and/or anti-CD28 antibodies, e.g., OKT-3, in the culture medium. In some embodiments, the stimulating agents include IL-2 and/or IL-15 and/or IL-7, for example, IL-2 at a concentration of at least about 10 units/mL.

In some embodiments, the activating conditions or agents include one or more agents (e.g., ligands) capable of activating the intracellular signaling domain of a TCR complex. In some aspects, the agent turns on or initiates TCR/CD3 intracellular signaling cascade in T cells. Such agents may include, for example, antibodies bound to solid supports (e.g., beads, including magnetic beads or microbeads), such as antibodies specific for TCR components and/or costimulatory receptors (e.g., anti-CD3, anti-CD28); and/or one or more cytokines. Optionally, the expansion method can further include a step of adding anti-CD3 and/or anti-CD28 antibodies to the culture medium (e.g., at a concentration of at least about 0.5 ng/ml). In some embodiments, the stimulating agents include IL-2 and/or IL-15 and/or IL-7, e.g., IL-2 at a concentration of at least about 10 units/mL, at least about 50 units/mL, at least about 100 units/mL or at least about 200 units/mL.

In some embodiments, the total duration of incubation with the activating agent is, for example, at or about between 1 hour and 96 hours, between 1 hour and 72 hours, between 1 hour and 48 hours, 4 hours and 36 hours between 8 hours and 30 hours or between 12 hours and 24 hours, such as at least 6 hours, 12 hours, 18 hours, 24 hours, 36 hours or 72 hours.

In some embodiments, the methods provided herein do not include further culturing or incubation, for example, not including an ex vivo expansion step, or including a significantly shorter ex vivo expansion step.

In some embodiments, the entire process of engineering the cells (e.g., selection and/or enrichment, incubation combined with activation and transduction and/or further culturing or incubation) is performed within following periods of time after a sample is obtained from a subject: no more than 9 days, no more than 8 days, no more than 7 days, no more than 6 days, no more than 5 days, no more than 4 days, no more than 3 days, no more than 2 days or no more than 1 day. It should be appreciated that this timing does not include any period of time during which the cells are subjected to cryopreservation.

In some embodiments of the methods provided herein, the engineered cells (e.g., the output composition or formulated composition) are administered to a subject immediately or shortly after transduction without significant ex vivo expansion. In some embodiments, the engineered cells can be administered immediately after the transduction step. In some embodiments, the engineered cells can be administered shortly after the activation transduction step under consitions that, for example, no significant ex vivo expansion or significantly shorter ex vivo expansion is performed compared with conventional methods (which may require significant in vitro activation, expansion and/or enrichment). For example, in some embodiments of the methods provided herein, the engineered cells can be administered within three days, two days, or one day of transduction. In some embodiments, the engineered cells can be administered within 48 hours, 36 hours, 24 hours, 20 hours, 16 hours, 12 hours, 8 hours, 4 hours, 2 hours, 1 hour or less of the activation and transduction steps. In some embodiments, the engineered cells are subjected to significantly shorter in vitro expansion, e.g., 48 hours, 36 hours, 24 hours, 20 hours, 16 hours, 12 hours, 8 hours, 4 hours, 2 hours, 1 hour or less.

In any such embodiment, the expansion and/or activation of the cells can be performed in vivo following exposure to the antigen. For example, the engineered cells is expanded in a subject following the administration of the cells. In some embodiments, the range, extent, or magnitude of in vivo expansion can be augmented, strenthened, or enhanced by a variety of methods that can modulate (e.g., increase) the expansion, proliferation, survival and/or efficacy of the administered cells (e.g., cells expressing recombinant receptors).

In some embodiments, such methods include methods involving administering engineered cells that are further modified with an agent (e.g., nucleic acid) to alter (e.g., increase or decrease) the expression or activity of a molecule, wherein the altered expression or activity will amplify, enhance or enhance the expansion, proliferation, survival and/or efficacy of the administered cells. In some embodiments, the expression of an agent (e.g., a nucleic acid) is inducible, repressible, regulatable, and/or user-controlled by, for example, the administration of an inducer or other regulatory molecules.

In some embodiments, such methods include methods involving the combined administration (e.g., simultaneous or sequential administration) with a drug or agent that can expand, strenthen, or enhance the expansion, proliferation, survival and/or efficacy of the administered cells (e.g., cells expressing recombinant receptors).

In some embodiments, the viral vector particle is a retroviral vector particle, such as a lentiviral particle, which contains a nucleic acid in the genome of the viral vector encoding recombinant and/or heterologous molecules (e.g., recombinant or heterologous proteins, such as recombinant and/or heterologous receptors, such as chimeric antigen receptors (CARs) or other antigen receptors). The genome of a viral vector particle typically includes sequences other than the nucleic acid encoding the recombinant molecule. Such sequences may include sequences that allow packaging of the genome into viral particles and/or sequences that facilitate expression of nucleic acids encoding recombinant receptors (e.g., CARs).

In some embodiments, the viral vector particle contains a genome derived from a retroviral genome-based vector (e.g., derived from a lentiviral genome-based vector). In some aspects of the provided viral vectors, a heterologous nucleic acid encoding a recombinant receptor (e.g., an antigen receptor, such as a CAR) is contained and/or located between 5′LTR and 3′LTR sequences of the vector genome.

In some embodiments, the viral vector genome is a lentiviral genome, such as an HIV-1 genome or a SIV genome. In some embodiments, these viral vectors are plasmid-based or virus-based and are configured to carry essential sequences for incorporation of foreign nucleic acids, so that said nucleic acids can be selected and transferred into host cells.

Non-limiting examples of lentiviral vectors include those derived from lentiviruses such as human immunodeficiency virus 1 (HIV-1), HIV-2, simian immunodeficiency virus (SIV), human T lymphotropic virus 1 (HTLV-1), HTLV-2 or Equine Infection Anemia Virus (E1AV). In some embodiments, the viral genomic vector may contain sequences of 5′ and 3′ LTRs of retroviruses (e.g., lentiviruses). In some aspects, the viral genome construct can contain sequences from 5′ and 3′ LTRs of the lentivirus, and in particular, can contain R and U5 sequences from 5′ LTR of the lentivirus and inactivated or self-inactivated 3′ LTR from the lentivirus. The LTR sequence can be that from any lentivirus of any species. For example, they can be LTR sequences from HIV, SIV, FIV or BIV. Typically, the LTR sequence is an HIV LTR sequence.

In some embodiments, the viral vector contains nucleic acid encoding a heterologous recombinant protein. In some embodiments, the heterologous recombinant molecule is or includes a recombinant receptor (e.g., a chimeric antigen receptor), a SB transposon (e.g., for gene silencing), a capsid-encapsulated transposon, a homoduplex strand nucleic acid (e.g., for genomic recombination) or a reporter gene (e.g., fluorescent protein, such as GFP) or luciferase).

In some embodiments, the viral vector contains a nucleic acid encoding a recombinant receptor and/or a chimeric receptor (e.g., a heterologous receptor protein). The recombinant receptors (e.g., heterologous receptors) can include antigen receptors, such as functional non-TCR antigen receptors, including chimeric antigen receptors (CARs) and other antigen-binding receptors, such as transgenic T cell receptors (TCRs). Receptors may also include other receptors, such as other chimeric receptors, such as receptors that bind to specific ligands and have transmembrane and/or intracellular signaling domains similar to those present in the CAR.

In some embodiments, the encoded recombinant antigen receptor (e.g., CAR) is a receptor capable of specifically binding to one or more ligands on a cell or disease to be targeted, such as cancer, infectious diseases, inflammatory or autoimmune diseases or other diseases.

In some embodiments, exemplary antigens are or include αvβ6 integrin (avb6 integrin), B cell maturation antigen (BCMA), B7-H3, B7-H6, carbonic anhydrase 9 (CA9, also known as CAIX or G250), cancer-testis antigen, cancer/testis antigen 1B (CTAG, also known as NY-ESO-1 and LAGE-2), Carcinoembryonic Antigen (CEA), Cyclin, Cyclin A2, CC Motif Chemokine Ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (tEGFR), type III epidermal growth factor receptor mutation (EGFR vIII), Epiglin 2 (EPG-2), Epiglin 40 (EPG-40), Ephrin B2, Ephrin Receptor A2 (EPHa2), Estrogen Receptor, Fc Receptor-like 5 (FCRL5; also known as Fc receptor homolog 5 or FCRH5), fetal acetylcholine receptor (fetal AchR), folate binding protein (FBP), folate receptor alpha, ganglioside GD2, O-acetylated GD2 (OGD2), ganglioside GD3, glycoprotein 100 (gp100), G protein coupled receptor 5D (GPCR5D), Her2/neu (receptor tyrosine kinase erb-B2), Her3 (erb-B3), Her4 (erb-B4), erbB dimer, human high molecular weight melanoma-associated antigen (HMW-MAA), hepatitis B surface antigen, human leukocyte antigen A1 (HLAA1), human leukocyte antigen A2 (HLA-A2), IL-22 receptor alpha (IL-22Ra), IL-13 receptor alpha 2 (IL-13Ra2), kinase insertion domain receptor (kdr), kappa light chain, L1 cell adhesion molecule (L1-CAM), L1-CAM CE7 epitope, leucine-rich repeat containing 8 family member A (LRRC8A), Lewis Y, melanoma-associated antigen (MAGE)-A1, MAGE-A3, MAGEA6, mesothelin, c-Met, Murine cytomegalovirus (CMV), mucin 1 (MUC1), MUC16, natural killer family 2 member D (NKG2D) ligand, melanin A (MART-1), neural cell adhesion molecule (NCAM), carcinoembryonic antigen, preferentially expressed melanoma antigen (PRAME), progesterone receptor, prostate-specific target antigen, prostate stem cell antigen (PSCA), prostate-specific membrane antigen (PSMA), receptor tyrosine kinase-like orphan receptor 1 (ROR1), survivin, trophoblast glycoprotein (TPBG, also known as 5T4), tumor-associated glycoprotein 72 (TAG72), vascular endothelial growth factor receptor (VEGFR), vascular endothelial growth factor Receptor 2 (VEGFR2), Wilms tumor 1 (WT-1), pathogen-specific target antigens or antigens associated with universal tags, and/or biotinylated molecules, and/or by HIV, HCV, HBV or other pathogens expressed molecule. In some embodiments, the antigen targeted by the receptor includes an antigen associated with B cell malignancies, such as any of a number of known B cell markers. In some embodiments, the antigen is or includes CD20, CD19, CD22, ROR1, CD45, CD21, CD5, CD33, Igκ, Igλ, CD79a, CD79b or CD30.

In some embodiments, exemplary antigens are orphan tyrosine kinase receptors ROR1, tEGFR, Her2, L1-CAM, CD19, CD20, CD22, mesothelin, CEA and hepatitis B surface antigen, antifolate receptor, CD23, CD24, CD30, CD33, CD38, CD44, EGFR, EGP-2, EGP-4, OEPHa2, ErbB2, 3 or 4, FBP, fetal acetylcholine receptor, GD2, GD3, HMW-MAA, IL-22R-α, IL-13R-α2, kdr, κ Light chain, Lewis Y, L1 cell adhesion molecule, MAGE-A1, mesothelin, MUC1, MUC16, PSCA, NKG2D ligand, NY-ESO-1, MART-1, gp100, carcinoembryonic antigen, ROR1, TAG72, VEGF-R2, carcinoembryonic antigen (CEA), prostate specific target antigen, PSMA, Her2/neu, estrogen receptor, progesterone receptor, ephrin B2, CD123, CS-1, c-Met, GD-2 and MAGE A3, CE7, Wilms tumor 1 (WT-1), cyclins (eg, cyclin A1 (CCNA1)), and/or biotinylated molecules, and/or molecules expressed by HIV, HCV, HBV, HPV and/or other pathogens and/or characteristic molecules of HIV, HCV, HBV, HPV and/or other pathogens, and/or molecules specific for HIV, HCV, HBV, HPV and/or other pathogens or carcinogenic forms thereof.

In some embodiments, the antigen is or includes a pathogen-specific or pathogen-expressed antigen. In some embodiments, the antigens are viral antigens (e.g., viral antigens from HIV, HCV, HBV, etc.), bacterial antigens, and/or parasite antigens.

In some embodiments, the antigen receptor (including CAR and recombinant TCR) and the preparation and introduction thereof include, for example, those described in: WO2015172339A1, WO2016008405A1, WO 2016086813A1, WO2016150400, WO2017032293A1, WO2017041749A1, WO2017080377A1, WO2018018958A1, WO2018108106A1, WO2018045811A1, WO 2018219299, WO 2018/210279, WO2019/024933, WO2019/114751, WO2019/114762, WO2019/149279, WO 2019/170147A1, WO 2019/210863, CN109385400A, CN109468279A, CN109880803A, CN 110438082 A, CN 110468105 A, WO2019/219029, WO 200014257, WO 2013126726, WO 2012/129514, WO 2014031687, WO 2013/166321, WO 2013/071154, WO 2013/123061, US 2002131960, US 2013287748, US 20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353 and 8,479,118, and EP 2537416, and/or those described in: Sadelain et al., Cancer Discov. April 2013, 3(4): 388-398; Davila et al., (2013) PLoS ONE 8(4): e61338; Turtle et al., Curr. Opin. Immunol., October 2012; 24(5): 633-39; Wu et al., Cancer, Mar. 18, 2012 (2): 160-75.

a. Chimeric Antigen Receptor

In some embodiments, the nucleic acid contained in the viral vector genome encodes a chimeric antigen receptor (CAR). CARs are typically genetically engineered receptors that have an extracellular ligand-binding domain, such as an extracellular portion containing an antibody or fragment thereof, and the extracellular ligand-binding domain is associated with one or more intracellular signaling components. In some embodiments, the chimeric antigen receptor includes a transmembrane domain and/or an intracellular domain linking the extracellular domain and the intracellular signaling domain. Such molecules typically mimic or approximate a signal sent by a native antigen receptor and/or a signal sent by the combination of such a receptor and a costimulatory receptor.

In some embodiments, the CAR is constructed with specificity for a particular marker, e.g., a marker expressed in a particular cell type targeted by adoptive therapy, such as a cancer marker and/or any of the antigens. Therefore, a CAR typically includes one or more antigen-binding fragments, domains or portions of an antibody, or one or more antibody variable domains and/or antibody molecules. In some embodiments, the CAR includes one or more antigen-binding portions of an antibody molecule, such as a variable heavy chain (VH) or antigen-binding portion thereof, or a single-chain antibody fragment (scFv) derived from a variable heavy chain (VH) and variable light chains (VL) of monoclonal antibodies (mAbs).

In some embodiments, engineered cells, such as T cells, are provided that express a CAR specific for a specific antigen (or marker or ligand), e.g., an antigen expressed on the surface of a specific cell type. In some embodiments, the antigen is a polypeptide. In some embodiments, it is a carbohydrate or other molecules. In some embodiments, the antigen is selectively expressed or overexpressed on cells of the disease, such as tumor cells or disease-causing cells, as compared with normal or non-targeted cells or tissues. In other embodiments, the antigen is expressed on normal cells and/or on engineered cells.

In certain embodiments, the recombinant receptor (e.g., a chimeric receptor) contains an intracellular signaling region that includes a cytoplasmic signaling domain or region (also interchangeably named as intracellular signaling domains or regions), such as cytoplasmic (intracellular) regions capable of inducing primary activation signals in T cells, for example, cytoplasmic signaling domains or regions of T cell receptor (TCR) components (e.g., CD3-zeta (CD3ζ) chain or the cytoplasmic signaling domain or region of (chain of a functional variant or signaling portion thereof); and/or the intracellular signaling region comprises cytoplasmic signaling domains or regions of an immunoreceptor tyrosine-based activation motif (ITAM).

In some embodiments, the chimeric receptor also contains an extracellular ligand binding domain that specifically binds to the ligand (e.g., antigen). In some embodiments, the chimeric receptor is a CAR, which contains an extracellular antigen recognition domain that specifically binds to an antigen. In some embodiments, the ligand (e.g., antigen) is a protein expressed on the surface of a cell. In some embodiments, the CAR is a TCR-like CAR and the antigen is a processed peptide antigen, such as a peptide antigen of an intracellular protein, which is, like the TCR, recognized on the surface of a cell in the context of major histocompatibility complex (MHC) molecules.

Exemplary antigen receptors, including CARs, and methods for engineering and introducing such receptors into cells include, for example, those described in: WO2015172339A1, WO2016008405A1, WO 2016086813A1, WO2016150400, WO2017032293A1, WO2017041749A1, WO2017080377A1, WO2018018958A1, WO2018108106A1, WO2018045811A1, WO 2018219299, WO 2018/210279, WO2019/024933, WO2019/114751, WO2019/114762, WO2019/149279, WO 2019/170147A1, WO 2019/210863, CN109385400A, CN109468279A, CN109880803A, CN 110438082 A, CN110468105 A, WO2019/219029, WO 200014257, WO2013126726, WO 2012/129514, WO2014031687, WO2013/166321, WO2013/071154, WO2013/123061, US 2002131960, US 2013287748, US 20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, 8,479,118 and EP 2537416 and/or those disclosed in Sadelain et al., Cancer Discov. April 2013, 3(4): 388-398; Davila et al., (2013) PLoS ONE 8(4): e61338; Turtle et al., Curr. Opin. Immunol., October 2012; 24(5): 633-39; Wu et al., Cancer, Mar. 18, 2012; (2): 160-75. In some aspects, antigen receptors include CARs as described in U.S. Pat. No. 7,446,190, and those described in WO/2014055668A1.

Examples of CARs include CARs as disclosed in any of the following publications, such as WO2015172339 A1, WO2016008405A1, WO2016086813A1, WO2016150400, WO2017032293A1, WO 2017041749A1, WO2017080377A1, WO2018018958A1, WO2018108106A1, WO2018045811 A1, WO 2018219299, WO2018/210279, WO2019/024933, WO2019/114751, WO2019/114762, WO2019/149279, WO2019/170147A1, WO 2019/210863, CN109385400A, CN109468279A, CN109880803A, CN 110438082 A, CN 110468105 A, WO2019/219029, WO 2014031687, U.S. Pat. Nos. 8,339,645, 7,446,179, US 2013/0149337, U.S. Pat. Nos. 7,446,190, 8,389,282; Kochenderfer et al., 2013, Nature Reviews Clinical Oncology, 10, 267-276 (2013); Wang et al., (2012) J. Immunother. 35(9): 689-701; and Brentjens et al., Sci Transl Med. 2013 5 (177) (also see WO 2014031687, U.S. Pat. Nos. 8,339,645, 7,446,179, US 2013/0149337, U.S. Pat. Nos. 7,446,190 and 8,389,282).

In some embodiments, the CAR is constructed to have specificity for a particular antigen (or a marker or ligand), e.g., an antigen expressed in a particular cell type targeted by an adoptive therapy (e.g., a cancer marker) and/or antigen intended to induce attenuated responses (e.g., antigens expressed on normal or non-diseased cell types). Therefore, the CAR typically includes one or more antigen-binding molecules, such as one or more antigen-binding fragments, domains or portions, or one or more antibody variable domains, and/or antibody molecules in its extracellular portion. In some embodiments, the CAR includes one or more antigen-binding portions of an antibody molecule, such as a single-chain antibody fragment (scFv) derived from variable heavy (VH) and variable light (VL) chains of a monoclonal antibody (mAb).

In some embodiments, the antibody or antigen-binding portion thereof is expressed on a cell as part of a recombinant receptor (e.g., an antigen receptor). The antigen receptors include functional non-TCR antigen receptors, such as chimeric antigen receptors (CARs). In general, CARs containing antibodies or antigen-binding fragments that exhibit TCR-like specificity against peptide-MHC complexes can also be named as TCR-like CARs. In some embodiments, in some aspects, the extracellular antigen-binding domain specific for the MHC-peptide complex of the TCR-like CAR is bound to one or more intracellular signaling components via a linker and/or one or more transmembrane domains. In some embodiments, such molecules can generally mimic or approximate a signal through a native antigen receptor (e.g., TCR), and mimic or approximate a signal optionally through such a receptor in combination with a costimulatory receptor.

In some embodiments, a recombinant receptor (e.g., a chimeric receptor, such as a CAR) includes a ligand binding domain that binds (e.g., specifically binds) to an antigen (or ligand). Antigens targeted by chimeric receptors include antigens expressed in the context of a disease, disorder or cell type targeted by an adoptive cell therapy. The disease and disorder include a proliferative, neoplastic and malignant disease, including a cancer and tumor, including a blood cancer, cancer of the immune system, such as lymphoma, leukemia, and/or myeloma, such as B-type leukemia, T-type leukemia, and myeloma Leukemia, lymphoma and multiple myeloma.

In some embodiments, the antigen (or ligand) is a polypeptide. In some embodiments, it is a carbohydrate or other molecules. In some embodiments, the antigen (or ligand) is selectively expressed or overexpressed on cells of a disease (e.g., tumor or disease-causing cells) as compared with normal or non-targeted cells or tissues. In other embodiments, the antigen is expressed on normal cells and/or on engineered cells.

In some embodiments, the CAR contains an antibody or antigen-binding fragment (e.g., scFv) that specifically recognizes an antigen, such as an intact antigen, expressed on the cell surface. In some embodiments, the antigen (or ligand) is a tumor antigen or a cancer marker. In some embodiments, the antigen (or ligand) is or includes αvβ6 integrin (avb6 integrin), B cell maturation antigen (BCMA), B7-H3, B7-H6, carbonic anhydrase 9 (CA9, also known as CAIX or G250), cancer-testis antigen, cancer/testis antigen 1B (CTAG, also known as NY-ESO-1 and LAGE-2), Carcinoembryonic Antigen (CEA), Cyclin, Cyclin A2, CC Motif Chemokine Ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (tEGFR), type III epidermal growth factor receptor mutation (EGFR vIII), Epiglin 2 (EPG-2), Epiglin 40 (EPG-40), Ephrin B2, Ephrin Receptor A2 (EPHa2), Estrogen Receptor, Fc Receptor-like 5 (FCRL5; also known as Fc receptor homolog 5 or FCRH5), fetal acetylcholine receptor (fetal AchR), folate binding protein (FBP), folate receptor α, ganglioside GD2, O-acetylated GD2 (OGD2), ganglioside GD3, glycoprotein 100 (gp100), G protein coupled receptor 5D (GPCR5D), Her2/neu (receptor tyrosine kinase erb-B2), Her3 (erb-B3), Her4 (erb-B4), erbB dimer, human high molecular weight melanoma-associated antigen (HMW-MAA), hepatitis B surface antigen, human leukocyte antigen A1 (HLAA1), human leukocyte antigen A2 (HLA-A2), IL-22 receptor alpha (IL-22Ra), IL-13 receptor alpha 2 (IL-13Ra2), kinase insertion domain receptor (kdr), kappa light chain, L1 cell adhesion molecule (L1-CAM), L1-CAM CE7 epitope, leucine-rich repeat containing 8 family member A (LRRC8A), Lewis Y, melanoma-associated antigen (MAGE)-A1, MAGE-A3, MAGEA6, mesothelin, c-Met, Murine cytomegalovirus (CMV), mucin 1 (MUC1), MUC16, natural killer family 2 member D (NKG2D) ligand, melanin A (MART-1), neural cell adhesion molecule (NCAM), carcinoembryonic antigen, preferentially expressed melanoma antigen (PRAME), progesterone receptor, prostate-specific target antigen, prostate stem cell antigen (PSCA), prostate-specific membrane antigen (PSMA), receptor tyrosine kinase-like orphan receptor 1 (ROR1), survivin, trophoblast glycoprotein (TPBG, also known as 5T4), tumor-associated glycoprotein 72 (TAG72), vascular endothelial growth factor receptor (VEGFR), vascular endothelial growth factor Receptor 2 (VEGFR2), Wilms tumor 1 (WT-1), pathogen-specific target antigens or antigens associated with universal tags, and/or biotinylated molecules, and/or by HIV, HCV, HBV or other pathogens expressed molecule. In some embodiments, the antigen targeted by the receptor includes an antigen associated with B cell malignancies, such as any of a number of known B cell markers. In some embodiments, the antigen is or includes CD20, CD19, CD22, ROR1, CD45, CD21, CD5, CD33, Igκ, Igλ, CD79a, CD79b or CD30.

In some embodiments, the antigen is or includes a pathogen-specific or pathogen-expressed antigen. In some embodiments, the antigens are viral antigens (e.g., viral antigens from HIV, HCV, HBV, etc.), bacterial antigens, and/or parasite antigens. In some embodiments, the CAR contains a TCR-like antibody, e.g., an antibody or antigen-binding fragment (e.g., scFv), which specifically recognizes an intracellular antigen (e.g., a tumor-associated antigen) present on the cell surface as an MHC-peptide complex. In some embodiments, an antibody or antigen-binding portion thereof that recognizes an MHC-peptide complex can be expressed on a cell as part of a recombinant receptor (e.g., an antigen receptor). The antigen receptors include functional non-TCR antigen receptors, such as chimeric antigen receptors (CARs). In general, CARs containing antibodies or antigen-binding fragments that exhibit TCR-like specificity against peptide-MHC complexes can also be named as TCR-like CARs.

In some embodiments, an antibody or antigen-binding portion thereof that specifically binds to an MHC-peptide complex can be produced by immunizing a host with an effective amount of an immunogen containing a particular MHC-peptide complex. In some cases, the peptide of the MHC-peptide complex is an epitope capable of binding to an antigen of MHC, e.g., a tumor antigen, such as a universal tumor antigen, a myeloma antigen, or other antigens as described below. In some embodiments, an effective amount of the immunogen is then administered to the host for eliciting an immune response, wherein the immunogen retains its three-dimensional form for a period sufficient to elicit an immune response against a three-dimensional presentation of the peptide in the binding groove of the MHC molecule. Serum collected from the host is then assayed to determine whether the desired antibodies recognizing the three-dimensional presentation of the peptide in the binding groove of the MHC molecule are produced. In some embodiments, the produced antibodies can be evaluated to confirm that the antibodies can discriminate between MHC-peptide complexes and MHC molecules alone, target peptides alone, and complexes of MHC and unrelated peptides. The desired antibody can then be isolated.

Single domain antibodies are antibody fragments comprising all or part of the heavy chain variable domain or all or part of the light chain variable domain of an antibody. In certain embodiments, the single domain antibody is a human single domain antibody. In some embodiments, the CAR comprises an antibody heavy chain domain that specifically binds an antigen, such as a marker of cancer or a cell surface antigen of a cell or disease (e.g., tumor cell or cancer cell) to be targeted, such as any target antigens described herein or known.

Antibody fragments can be prepared by various techniques including, but not limited to, proteolytic digestion of intact antibodies and production by recombinant host cells. In some embodiments, the antibody is a recombinantly produced fragment, such as a fragment comprising an arrangement that does not occur in nature (such as those having two or more antibody regions or chains joined by a synthetic linker (e.g., a peptide linker)), and/or fragments that may not be produced by enzymatic digestion of naturally occurring intact antibodies. In some embodiments, the antibody fragment is an scFv.

In some embodiments, a chimeric antigen receptor (including a TCR-like CAR) includes an extracellular portion comprising an antibody or antibody fragment. In some embodiments, the antibody or fragment includes an scFv. In some aspects, a chimeric antigen receptor includes an antibody or fragment-containing extracellular portion and an intracellular signaling region. In some embodiments, the intracellular signaling region comprises an intracellular signaling domain. In some embodiments, the intracellular signaling domain is or comprises a primary signaling domain, a signaling domain capable of inducing a primary activation signal in a T cell, a signaling domain of a T cell receptor (TCR) component and/or a signaling domain comprising an immunoreceptor tyrosine-based activation motif (ITAM).

In some embodiments, the extracellular portion of the CAR (e.g., the antibody portion thereof) further comprises a spacer, e.g., a spacer region between the antigen recognition component (e.g., the scFv) and the transmembrane domain. The spacer may be or include at least a portion of an immunoglobulin constant region or a variant or modified form thereof, e.g., a hinge region, such as an IgG4 hinge region, and/or a CH1/CL and/or Fc region. In some embodiments, the recombinant receptor further comprises a spacer and/or hinge region. In some embodiments, the constant region or portion is that of a human IgG, such as IgG4 or IgG1. In some aspects, the portion of the constant region serves as a spacer region between the antigen recognition component (e.g., scFv) and the transmembrane domain.

In some embodiments, the spacer can be or include at least a portion of an immunoglobulin constant region or a variant or modified form thereof, such as a hinge region (e.g., an IgG4 hinge region), and/or a CH1/CL and/or Fc region. In some embodiments, the recombinant receptor further comprises a spacer and/or hinge region. In some embodiments, the constant region or portion is that of a human IgG, such as IgG4 or IgG1. In some aspects, the portion of the constant region serves as a spacer region between the antigen recognition component (e.g., scFv) and the transmembrane domain. The length of the spacer can provide enhanced cellular reactivity upon antigen binding, compared with the absence of the spacer. In some embodiments, the spacer region has about 12 or fewer amino acids, about 119 or fewer amino acids, or about 229 or fewer amino acids. Exemplary spacers include an IgG4 hinge alone, an IgG4 hinge linked to the CH2 and CH3 domains, or an IgG4 hinge linked to the CH3 domain. An extracellular ligand binding domain (e.g., an antigen recognition domain) is typically linked to one or more intracellular signaling components, which, in the case of a CAR, mimic an activated signaling component via an antigen receptor complex (e.g., a TCR complex) and/or a signal transduced through another cell surface receptor. In some embodiments, the transmembrane domain connects the extracellular ligand binding domain and the intracellular signaling domain. In some embodiments, the antigen binding component (e.g., antibody) is linked to one or more transmembrane and intracellular signaling regions. In some embodiments, the CAR includes a transmembrane domain fused to an extracellular domain. In one embodiment, a transmembrane domain that is naturally associated with one of the domains in a receptor (e.g., a CAR) is used. In some cases, the transmembrane domains are selected or modified by amino acid substitutions to avoid binding of such domains to transmembrane domains of the same or different surface membrane proteins, thereby minimizing the interaction with other members of the receptor complex.

In some embodiments, the transmembrane domain is derived from natural or synthetic sources. When derived from natural sources, in some aspects, the domain can be derived from any membrane-bound or transmembrane protein. The transmembrane region includes those derived from (i.e., including at least one or more transmembrane regions of): α, β or ζ chains of T cell receptors, CD28, CD3ε, CD45, CD4, CD5, CD8, CD9, CD16, CD22, CD33, CD37, CD64, CD80, CD86, CD134, CD137 or CD154. In some embodiments, the transmembrane domain is derived from synthetic sources. In some aspects, the synthetic transmembrane domain comprises predominantly hydrophobic residues, such as leucine and valine. In some aspects, a triplet of phenylalanine, tryptophan, and valine will be found at each end of the synthetic transmembrane domain. In some embodiments, the linking is achieved through a linker, spacer, and/or one or more transmembrane domains.

In some embodiments, a short oligopeptide or polypeptide linker (e.g., a linker between 2 and 10 amino acids in length, such as a linker containing glycine and serine, e.g., a glycine-serine doublet) is present and forms the connection between the transmembrane domain and the cytoplasmic signaling domain of a CAR.

A recombinant receptor (e.g., CAR) typically includes at least one or more intracellular signaling components. In some embodiments, the receptor includes an intracellular component of a TCR complex, such as TCR CD3 chain mediating T cell activation and cytotoxicity, e.g., CD3ζ chain. Therefore, in some aspects, the antigen binding moiety is linked to one or more cell signaling modules. In some embodiments, the cell signaling module includes a CD3 transmembrane domain, CD3 intracellular signaling domain, and/or other CD transmembrane domains. In some embodiments, the receptor (e.g., CAR) also includes a portion of one or more additional molecules (e.g., Fc receptor γ, CD8, CD4, CD25, or CD16). For example, in some aspects, a CAR or other chimeric receptor includes a chimeric molecule between CD3-ζ (CD3-ζ) or Fc receptor γ and CD8, CD4, CD25, or CD16.

In some embodiments, upon the attachment of a CAR or other chimeric receptors, the cytoplasmic domains and/or regions or intracellular signaling domains and/or regions of the receptor activate at least one of the normal effector functions or responses of immune cells (e.g., T cells engineered to express the CAR). For example, in some cases, the CAR induces T cell function, such as cytolytic activity or T helper activity, such as secretion of cytokines or other factors. In some embodiments, a truncated portion of the intracellular signaling domain of the antigen receptor component or co-stimulatory molecule (e.g., if it transduces effector function signals) is used to replace the complete immunostimulatory chain. In some embodiments, the intracellular signaling region (e.g., comprising one or more intracellular signaling domains) comprises the cytoplasmic sequence of the T cell receptor (TCR) and, in some aspects, further comprises a co-receptor (which acts in parallel with such receptors in the natural context to initiate signal transduction upon antigen receptor engagement) and/or any derivatives or variants of such molecules, and/or any synthetic sequences having the same functional capability.

In the case of native TCRs, not only signaling through the TCR, but also costimulatory signals are often required for the full activation. Therefore, in some embodiments, to facilitate the full activation, components for generating secondary or costimulatory signals are also included in a CAR. In other embodiments, the CAR does not include components for generating a costimulatory signal. In some aspects, the additional CAR is expressed in the same cell and provides components for generating secondary or costimulatory signals.

T cell activation is described, in some aspects, as being mediated by at least two classes of cytoplasmic signaling sequences: those that initiate antigen-dependent primary activation through the TCR (primary cytoplasmic signaling sequences), and those that act in an antigen-independent manner to provide secondary or costimulatory signals (secondary cytoplasmic signaling sequences). In some aspects, the CAR includes one or both of such signaling components.

In some aspects, the CAR includes a primary cytoplasmic signaling sequence that modulates primary activation of the TCR complex. Primary cytoplasmic signaling sequences that act in a stimulatory manner may contain signaling motifs (known as immunoreceptor tyrosine-based activation motif or ITAM). Examples of ITAMs containing primary cytoplasmic signaling sequences include those derived from: TCR or CD3ζ, FcRγ, FcRβ, CD3γ, CD3δ, CD3ε, CD8, CD22, CD79a, CD79b and CD66d. In certain embodiments, ITAMs containing primary cytoplasmic signaling sequences include those derived from TCR or CD3ζ, FcRγ or FcRβ. In some embodiments, the cytoplasmic signaling molecule in the CAR contains a cytoplasmic signaling domain derived from CD3ζ.

In some embodiments, the CAR includes a signaling domain and/or a transmembrane portion of a costimulatory receptor (e.g., CD28, 4-1BB, OX40, CD27, DAP10, and ICOS). In some aspects, the same CAR includes an activation or signaling region and a costimulatory component.

In some embodiments, the activation domain is included in one CAR, and the costimulatory component is provided by another CAR recognizing another antigen. In some embodiments, the CAR comprises an activating or stimulating CAR and a costimulatory CAR expressed on the same cell (see WO 2014/055668). In some aspects, the CAR is a stimulatory or activating CAR; and in other aspects, it is a costimulatory CAR. In some embodiments, the cell further comprises an inhibitory CAR (iCAR, see Fedorov et al., Sci. Transl. Medicine, 5(215) (December 2013), such as a CAR recognizing a different antigen, wherein the activation signal delivered by the CAR recognizing the first antigen is reduced or inhibited by the binding of the inhibitory CAR to its ligand, for example, to reduce off-target effects.

In some embodiments, the intracellular signaling domain comprises a CD28 transmembrane and signaling domain linked to the CD3 intracellular domain. In some embodiments, the intracellular signaling domain comprises a chimeric CD28 and CD137 costimulatory domain linked to the CD3 intracellular domain.

In some embodiments, the intracellular signaling domain of CD8+ cytotoxic T cells is the same as the intracellular signaling domain of CD4+ helper T cells. In some embodiments, the intracellular signaling domain of CD8+ cytotoxic T cells is different from the intracellular signaling domain of CD4+ helper T cells.

In some embodiments, a CAR encompasses one or more (e.g., two or more) costimulatory domains and an activation domain (e.g., a primary activation domain) in the cytoplasmic portion. Exemplary CARs comprise the intracellular components of CD3-ζ, CD28 and 4-1BB.

In some embodiments, the one or more recombinant receptors (e.g., CARs) encoded by the one or more nucleic acids within the provided viral vectors also include one or more markers, for example, for confirming the transduction or engineering of cells in which the receptor is to be expressed, and/or selecting of cells in which one or more molecules encoded by a polynucleotide are expressed, and/or targeting. In some aspects, such markers can be encoded by different nucleic acids or polynucleotides, which can also be introduced during the genetic engineering process, typically by the same method (for example, transduced by any of the methods provided herein, for example, by the same vector or the same type of vector).

In some aspects, the marker (e.g., a transduction marker) is a protein and/or a cell surface molecule. Exemplary markers are truncated variants of naturally occurring (e.g., endogenous) markers (e.g., naturally occurring cell surface molecules).

In some instances, the CAR is named as the first-, second-, and/or third-generation of CAR. In some aspects, the first-generation of CAR is a CAR that provides only a CD3 chain-induced signal upon antigen binding; in some aspects, the second-generation of CAR is a CAR that provides such a signal and a costimulatory signal, for example, a CAR comprising an intracellular signaling domain from a costimulatory receptor (e.g., CD28 or CD137); and in some aspects, the third-generation of CAR is a CAR comprising multiple costimulatory domains of different costimulatory receptors.

In some embodiments, a chimeric antigen receptor includes an extracellular ligand binding portion (e.g., an antigen binding portion, such as an antibody or fragment thereof) and an intracellular domain. In some embodiments, the antibody or fragment comprises a scFv or a single domain VH antibody, and the intracellular domain contains ITAM. In some aspects, the intracellular signaling domain comprises the signaling domain of the (chain of the CD3-zeta (CD3ζ) chain. In some embodiments, the chimeric antigen receptor includes a transmembrane domain linked and/or disposed between the extracellular domain and the intracellular signaling region or domain.

In some aspects, the transmembrane domain contains the transmembrane portion of CD28. The extracellular domain and transmembrane portion can be linked directly or indirectly. In some embodiments, the extracellular domain and the transmembrane portion are linked by a spacer (such as any spacers described herein). In some embodiments, the chimeric antigen receptor contains an intracellular domain of a T cell costimulatory molecule, such as between the transmembrane domain and the intracellular signaling domain. In some aspects, the T cell costimulatory molecule is CD28 or 4-1BB.

In some embodiments, the CAR contains an antibody (e.g., an antibody fragment), a transmembrane domain that is or contains a transmembrane portion of CD28 or a functional variant thereof, and a intracellular signaling domain that contains a signaling portion of CD28 or a functional variant thereof and a signaling portion of CD3ζ or a functional variant thereof. In some embodiments, the CAR contains an antibody, such as an antibody fragment, a transmembrane domain (which is a transmembrane portion of CD28 or a functional variant thereof or a transmembrane portion containing CD28 or a functional variant thereof), and an intracellular signaling domain containing a signaling portion of 4-1BB or a functional variant thereof and a signaling portion of CD3ζ or a functional variant thereof. In some embodiments, the receptor further comprises a spacer that contains a portion of an Ig molecule (e.g., a human Ig molecule, such as an Ig hinge, such as an IgG4 hinge), such as a spacer only containing a hinge.

In some embodiments, the transmembrane domain of the receptor (e.g., CAR) is the transmembrane domain of human CD28 or a variant thereof, e.g., transmembrane domain of human CD28 (Accession No: P10747.1) with 27 amino acids.

In some embodiments, the chimeric antigen receptor contains the intracellular domain of a T cell costimulatory molecule. In some aspects, the T cell costimulatory molecule is CD28 or 4-1BB.

In some embodiments, the intracellular domain comprises the intracellular costimulatory signaling domain of human CD28, or a functional variant or portion thereof, e.g., a domain thereof with 41 amino acids, and/or a domain having LL to GG substitution at positions 186-187 of the native CD28 protein.

In some embodiments, the intracellular signaling region and/or domain comprises a human CD3 chain, optionally a CD3ζ stimulatory signaling domain, or a functional variant thereof, e.g., a cytoplasmic domain of isotype 3 of human CD3ζ with 112 AA (Accession No: P20963.2) or CD3ζ signaling domain as described in U.S. Pat. No. 7,446,190 or U.S. Pat. No. 8,911,993.

In some embodiments, a CAR includes: an extracellular ligand binding portion, such as an antigen binding portion, such as an antibody or fragment thereof, including sdAbs and scFv, that specifically binds an antigen, such as an antigen described herein; a spacer, such as any spacer containing an Ig hinge; a transmembrane domain that is part of CD28 or a variant thereof; an intracellular signaling domain that contains a signaling portion of CD28 or a functional variant thereof; and a signaling portion of CD3ζ signaling domain or a functional variant thereof. In some embodiments, a CAR includes: an extracellular ligand binding portion, such as an antigen binding portion, such as an antibody or fragment thereof, including sdAb and scFv, that specifically binds an antigen, such as an antigen described herein; a spacer, such as any spacer containing an Ig hinge; a transmembrane domain that is part of CD28 or a variant thereof; an intracellular signaling domain that contains a signaling portion of 4-1BB or a functional variant thereof; and a signaling portion of CD3ζ signaling domain or a functional variant thereof.

b. T Cell Receptor (TCR)

In some embodiments, the one or more recombinant molecules encoded by the one or more nucleic acids are or include recombinant T cell receptors (TCR). In some embodiments, the recombinant TCR is specific for an antigen, typically an antigen present on a target cell, such as a tumor-specific target antigen, an antigen expressed on a particular cell type associated with autoimmune or inflammatory diseases, or antigens derived from viral or bacterial pathogens. In some embodiments, engineered cells, e.g., T cells, are provided that express TCRs or antigen-binding portions thereof, that recognize peptide epitopes of target polypeptides (e.g., antigens of tumor, virus, or autoimmune proteins) or T cell epitopes.

In some embodiments, a “T cell receptor” or “TCR” is a molecule or an antigen-binding portion thereof containing variable α and β chains (also named as TCRα and TCRβ, respectively) or variable γ and δ chains (also named as TCRα and TCRβ, respectively), which is capable of specifically binding to a peptide bound to an MHC molecule. In some embodiments, the TCR is in an αβ form. In general, TCRs in the αβ and γδ forms are generally structurally similar, but the T cells expressing them can have different anatomical locations or functions.

Unless otherwise stated, the term “TCR” should be understood to encompass an entire TCR as well as antigen-binding portions or antigen-binding fragments thereof. In some embodiments, the TCR is an intact or full-length TCR, including TCR in the αβ form or the γδ form. In some embodiments, the TCR is an antigen-binding portion that is less than a full-length TCR but binds to a specific peptide bound in an MHC molecule (e.g., binding to an MHC-peptide complex). In some cases, an antigen-binding portion or fragment of a TCR may contain only a portion of the structural domain of a full-length or intact TCR, but still be capable of binding a peptide epitope (e.g., an MHC-peptide complex) that binds to the intact TCR. In some cases, the antigen binding portion contains variable domains of a TCR (e.g., the variable α chain and variable β chain of a TCR) sufficient to form a binding site for binding to a particular MHC-peptide complex. Typically, the variable chain of a TCR contains complementarity determining regions involved in the recognition of peptides, MHCs and/or MHC-peptide complexes.

In some embodiments, the variable domains of a TCR contain hypervariable loops or complementarity determining regions (CDRs), which are typically the major contributors to antigen recognition and binding capacity and specificity. In some embodiments, the CDRs of a TCR, or a combination thereof, form all or substantially all of the antigen binding site of a given TCR molecule.

In some embodiments, the TCR chain contains a transmembrane domain. In some embodiments, the transmembrane domain is positively charged.

In some embodiments, the TCR can be a heterodimer of two chains, α and β (or optionally γ and δ), or it can be a single-chain TCR construct.

In some embodiments, the TCR contains sequences corresponding to transmembrane sequences. In some embodiments, the TCR does contain sequences corresponding to cytoplasmic sequences. In some embodiments, the TCR is capable of forming a TCR complex with CD3. In some embodiments, any TCR (including dTCR or scTCR) can be linked to a signaling domain that produces an active TCR on the surface of a T cell. In some embodiments, the TCR is expressed on the surface of the cell.

In some embodiments, one or more nucleic acids encoding TCRs (e.g., α and β chains) can be amplified by PCR, cloning, or other suitable methods, and cloned into a suitable expression vector. The expression vector can be any suitable recombinant expression vectors and can be used to transform or transfect any suitable hosts. Suitable vectors include those designed for propagation and amplification or for expression or both, such as plasmids and viruses.

In some embodiments, the vector may be of the following series: pUC series (Fermentas Life Sciences), pBluescript series (Stratagene, La Jolla, CA), pET series (Novagen, Madison, WI), pGEX series (Pharmacia Biotech), Uppsala, Sweden) or the pEX series (Clontech, Palo Alto, CA). In some cases, phage vectors, such as λG10, λGT11, λZa pII (Stratagene), λEMBL4 and λNM1149 can also be used. In some embodiments, plant expression vectors can be used and include pBI01, pBI101.2, pBI101.3, pBI121 and pBIN19 (Clontech). In some embodiments, animal expression vectors include pEUK-Cl, pMAM, and pMAMneo (Clontech). In some embodiments, viral vectors, such as retroviral vectors, are used.

In some embodiments, the recombinant expression vector can be prepared using standard recombinant DNA techniques. In some embodiments, the vector may contain regulatory sequences, such as transcriptional and translational initiation and termination codons, that are specific to the type of host (e.g., bacterial, fungal, plant, or animal) into which the vector is introduced. In some embodiments, the vector may contain a non-native promoter operably linked to a nucleotide sequence encoding a TCR or antigen binding portion (or other MHC-peptide binding molecule). In some embodiments, the promoter may be a non-viral promoter or a viral promoter, such as cytomegalovirus (CMV) promoter, SV40 promoter, RSV promoter, and the promoter found in the long terminal repeats of murine stem cell virus.

In some embodiments, cells and methods include multi-targeting strategies, such as expressing two or more genetically engineered receptors on a cell, each receptor recognizing the same or a different antigen, and typically each comprising a different intracellular signaling components.

In some embodiments of the methods and compositions provided herein, nucleic acid sequences encoding recombinant receptors (e.g., antigen receptors, e.g., CARs) contained in the viral vector genome are operably linked to other genetic elements (e.g., transcriptional regulatory sequences, including promoters or enhancers) in a functional relationship to modulate the expression of the target sequence in a specific manner. In certain instances, such transcriptional regulatory sequences are those that are temporally and/or spatially regulated in activity. Expression control elements that can be used to regulate the expression of components are known and include, but not limited to, inducible promoters, constitutive promoters, secretion signals, enhancers, and other regulatory elements. In some embodiments, the nucleic acid sequences contained in the viral vector genome contain multiple expression control elements that control different encoded components, such as different receptor components and/or signaling components, so that the expression, function, and/or activity of recombinant receptors and/or engineered cells (e.g., cells expressing engineered receptors) can be modulated, which are, for example, inducible, repressible, regulatable, and/or user-controlled. In some embodiments, one or more vectors may contain one or more nucleic acid sequences containing one or more expression control elements and/or one or more encoded components, so that the nucleic acid sequences together can modulate the expression, activity, and/or function of an encoded component (e.g., a recombinant receptor) or engineered cell.

In some embodiments, the nucleic acid sequence encoding a recombinant receptor (e.g., an antigen receptor, e.g., a CAR) is operably linked to an internal promoter/enhancer regulatory sequence. The used promoter can be a constitutive, tissue-specific, inducible promoter and/or available under appropriate conditions to direct high-level expression of the introduced DNA segments. Promoters can be heterologous or endogenous. In some embodiments, promoters and/or enhancers are produced synthetically. In some embodiments, promoters and/or enhancers are generated using recombinant cloning and/or nucleic acid amplification techniques.

In some cases, the nucleic acid sequence encoding the recombinant receptor contains a signal sequence encoding a signal peptide. In some aspects, the signal sequence can encode a signal peptide derived from a native polypeptide. In other aspects, the signal sequence can encode a heterologous or non-native signal peptide. In some cases, the nucleic acid sequence encoding a recombinant receptor (e.g., a chimeric antigen receptor (CAR)) contains a signal sequence encoding a signal peptide.

In some embodiments, the polynucleotide encoding the recombinant receptor contains at least one operably linked promoter to control the expression of the recombinant receptor. In some instances, the polynucleotide contains two, three or more operably linked promoters to control expression of the recombinant receptor.

In certain instances where the nucleic acid molecule encodes two or more different polypeptide chains (e.g., recombinant receptor and marker), each polypeptide chain may be encoded by a separate nucleic acid molecule. For example, two separate nucleic acids are provided, and each can be individually transferred or introduced into a cell for the expression in the cell. In some embodiments, the nucleic acid encoding the recombinant receptor and the nucleic acid encoding the marker are operably linked to identical promoters, and optionally separated through an internal ribosome entry site (IRES) or a nucleic acid encoding a self-cleaving peptide or a peptide leading to ribosome skipping (which is optionally T2A, P2A, E2A or F2A). In some embodiments, the nucleic acid encoding the marker and the nucleic acid encoding the recombinant receptor are operably linked to two different promoters. In some embodiments, the nucleic acid encoding the marker and the nucleic acid encoding the recombinant receptor are present in or inserted at different locations within the genome of the cell. In some embodiments, a polynucleotide encoding a recombinant receptor is introduced into a composition comprising cultured cells by, for exmple, retroviral transduction, transfection, or transformation.

In some embodiments, the oligonucleotide primer comprises a tag, wherein the tag is not specific to the target sequence. Such labels may be named as generic labels or generic marks. In some cases, the method includes labeling the target sequence or fragment thereof in the sample with a tag that is non-specific for the target sequence. In some cases, the tags are not specific for sequences on human chromosomes. Alternatively or additionally, the method includes contacting the sample with a tag and at least one oligonucleotide primer comprising a sequence corresponding to the target sequence, wherein the tag is separated from the oligonucleotide primer. In some cases, the tag is incorporated into the amplification product by extending the oligonucleotide primer after the hybridization of the oligonucleotide primer to the target sequence. Tags can be oligonucleotides, small molecules or peptides. In some embodiments, the marker is a transduction marker or surrogate marker. The transduction marker or surrogate marker can be used to detect cells into which a polynucleotide (e.g., a polynucleotide encoding a recombinant receptor) has been introduced. In some embodiments, the transduction marker can indicate or confirm the modification of the cell. In some embodiments, the surrogate marker is a protein prepared for co-expression with a recombinant receptor (e.g., CAR) on the cell surface. In certain embodiments, such surrogate marker is a surface protein that have been modified to have little or no activity. In some embodiments, the surrogate marker is encoded by the same polynucleotide encoding the recombinant receptor. In some embodiments, a nucleic acid sequence encoding the recombinant receptor is operably linked to a nucleic acid sequence encoding the marker, and optionally separated through an internal ribosome entry site (IRES) or a nucleic acid encoding a self-cleaving peptide or a peptide leading to ribosome skipping (which is optionally T2A, P2A, E2A or F2A). In some cases, an extrinsic marker gene can be used in combination with engineered cells to allow detection or selection of cells, and in some cases can also be used to promote cell suicide.

In some embodiments, a promoter and/or enhancer may be a promoter and/or enhancer with which the nucleic acid sequence is naturally associated, and may be obtained by, for example, isolating 5′ non-coding sequences located upstream of the coding segment and/or exon.

In some embodiments, the promoter may be a tissue-specific promoter and/or a target cell-specific promoter.

In some embodiments, regulatory elements can include regulatory elements and/or systems that allow for regulatable expression and/or activity of recombinant receptors (e.g., CARs). In some embodiments, regulatable expression and/or activity is achieved by configuring recombinant receptors to contain or be controlled by specific regulatory elements and/or systems.

Preparation of viral vector particles Viral vector genomes are usually constructed in plasmid form, which can be transfected into packaging or production cell lines. Retroviral particles whose genomes contain an RNA copy of the viral vector genome can be produced by using any of a variety of known methods. In some embodiments, at least two components are involved in preparing the virus-based gene delivery system: first, a packaging plasmid, including the structural proteins and enzymes necessary to generate the viral vector particles, and second, the viral vector itself, i.e., the genetic material to be transferred. Biosafety safeguards can be incorporated when designing one or both of these components.

In some embodiments, the packaging plasmid may contain all retroviral (e.g., HIV-1) proteins except for the envelope proteins (Naldini et al., 1998). In some embodiments, lentiviral vectors (e.g., HIV-based lentiviral vectors) contain only the genes of three parental viruses: gag, pol, and rev, which reduces or eliminates the possibility of wild-type virus remodeling by recombination.

In some embodiments, the viral vector genome is introduced into a packaging cell line containing all components required to package viral genomic RNA transcribed from the viral vector genome into viral particles.

In some embodiments, packaging cell lines are transfected with one or more plasmid vectors containing the components required to produce the particles. In some embodiments, plasmids containing the viral vector genome (including LTR, cis-acting packaging sequences, and target sequences, i.e., nucleic acids encoding antigen receptors (e.g., CARs)); and one or more helper plasmids encoding viral enzymes and/or structural components (such as Gag, pol and/or rev) are used to transfect packaging cell lines.

In some embodiments, the packaging cell line provides the components required for trans action to package viral genomic RNAs into lentiviral vector particles, including viral regulatory and structural proteins. In some embodiments, the packaging cell line can be any cell line capable of expressing lentiviral proteins and producing functional lentiviral vector particles. In some aspects, suitable packaging cell lines include 293 (ATCC CCL X), 293T, HeLA (ATCC CCL 2), D17 (ATCC CCL 183), MDCK (ATCC CCL34), BHK (ATCC CCL-10) and Cf2Th (ATCC CRL 1430) cells.

In some embodiments, the viral vector and packaging plasmid and/or helper plasmid are introduced into the packaging cell line by transfection or infection. Packaging cell lines produce viral vector particles containing the viral vector genome. Methods for transfection or infection are well known. Non-limiting examples include calcium phosphate, DEAE-dextran and lipofection methods, electroporation and microinjection.

In some embodiments, retroviral vectors, e.g., lentiviral vectors, can be produced in packaging cell lines (e.g., the exemplary HEK 293T cell line) by introducing plasmids to allow production of lentiviral particles. In some embodiments, the packaging cells are transfected and/or contain polynucleotides encoding gag and pol, and polynucleotides encoding recombinant receptors (e.g., antigen receptors, such as CAR). In some embodiments, the packaging cell line is optionally and/or additionally transfected with and/or contains a polynucleotide encoding a rev protein. In some embodiments, packaging cell lines are optionally and/or additionally transfected with and/or contain a polynucleotide encoding a non-native envelope glycoprotein (e.g., VSV-G). In some such embodiments, about two days after transfection of cells (e.g., HEK 293T cells), the cell supernatant contains recombinant lentiviral vector that can be recovered and titered.

The recovered and/or produced retroviral vector particles can be used to transduce target cells using methods as described. Once in the target cell, the viral RNA is reverse-transcribed, enters into the nucleus and stably integrated into the host genome. Expression of recombinant proteins (e.g., antigen receptors, such as CAR) can be detected one or two days after viral RNA integration.

### Incubation

In some embodiments, provided methods involve methods of transducing cells by contacting (e.g., incubating) an input composition comprising a plurality of cells with (1) viral particles. In some embodiments, the input composition comprises primary cells obtained from a subject, e.g., cells enriched and/or selected from the subject.

In some embodiments, the input composition comprises primary cells obtained from a subject. In some aspects, the sample is a whole blood sample, a buffy coat sample, a peripheral blood mononuclear cell (PBMC) sample, an unfractionated T cell sample, a lymphocyte sample, a leukocyte sample, an apheresis product, or a leukapheresis product.

In some embodiments, a sample containing primary cells is contacted with or contains serum or plasma ex vivo at the following concentrations prior to the selection and/or transduction of cells: at least or at least about 10% (v/v), at least or at least about 15% (v/v), at least or at least about 20% (v/v), at least or at least about 25% (v/v), at least or at least about 30% (v/v), at least or at least about 35% (v/v), at least or at least about 40% (v/v), or at least or at least about 50%. In some embodiments, the sample contains serum or plasma at or approximately at a concentration of about or at least about 25%, 26%, 27%, 28%, 29%, 30%, 31%, 32%, 33%, 34% or 35% (v/v). In some embodiments, the serum or plasma is of human. In some embodiments, the serum or plasma is autologous to a subject. In some embodiments, the sample containing primary cells is contacted with or contains an anticoagulant prior to the selection and/or transduction of cells. In some embodiments, the anticoagulant is or contains free citrate ions, e.g., the anticoagulant citrate dextrose solution, solution A (ACD-A).

In some embodiments, the sample is maintained at a temperature of 2° C. to 8° C. for up to 48 hours, e.g., up to 12 hours, 24 hours, or 36 hours, prior to the selection and/or transduction of cells.

In some embodiments, the infusion composition comprises and/or is enriched in T cells including CD4+ and/or CD8+ T cells. In some aspects, the enrichment can be performed by an affinity-based selection by incubating primary cells with one or more selection or affinity reagents that specifically bind to cell surface molecules expressed on the primary cell subpopulation, thereby enriching primary cells based on binding to selection reagents. In some embodiments, the enrichment can be performed by incubating cells with antibody-coated particles (e.g., microbeads, polymeric nanomatrixes).

In some embodiments, the input composition comprises greater than or greater than about 75%, 80%, 85%, 90%, 95%, or more T cells obtained from a subject's sample. In some aspects, prior to incubation, no more than 5%, 10%, 20%, 30%, or 40% of the T cells in the input composition are activated cells expressing a surface marker selected from the group consisting of HLA-DR, CD25, CD69, CD71, CD40L and 4-1BB; comprising intracellular expression of cytokines selected from IL-2, IFN-γ, or TNF-α; in the G1 phase or later of the cell cycle, and/or capable of proliferation.

In some embodiments, the input composition may comprise one or more cytokines during or during at least a portion of the incubation and/or contacting. In some embodiments, the cytokine is selected from IL-2, IL-7, or IL-15. In some embodiments, the cytokine is a recombinant cytokine. In some embodiments, the concentration of the cytokine in the input composition is independently 1 IU/mL to 1500 IU/mL, e.g., 1 IU/mL to 100 IU/mL, 2 IU/mL to 50 IU/mL, 5 IU/mL to 10 IU/mL, 10 IU/mL to 500 IU/mL, 50 IU/mL to 250 IU/mL or 100 IU/mL to 200 IU/mL, 50 IU/mL to 1500 IU/mL, 100 IU/mL to 1000 IU/mL or 200 IU/mL to 600 IU/mL. In some embodiments, the concentration of the cytokine in the input composition is independently at least or at least about 1 IU/mL, 5 IU/mL, 10 IU/mL, 50 IU/mL, 100 IU/mL, 200 IU/mL, 500 IU/mL, 1000 IU/mL or 1500 IU/mL. In some aspects, an agent capable of activating the intracellular signaling domain of a TCR complex (e.g., anti-CD3 and/or anti-CD28 antibodies) can also be included during or during at least a portion of the incubation or after the incubation.

In some embodiments, the input composition may comprise serum during or during at least a portion of the incubation and/or contacting. In some embodiments, the serum is human serum. In some embodiments, the serum is present in the input composition at a concentration of 0.5% to 25% (v/v), 1.0% to 10% (v/v), or 2.5% to 5.0% (v/v) (inclusive).

In some embodiments, the input composition is free and/or substantially free of serum during or during at least a portion of the incubation and/or contacting. In some embodiments, the input composition is incubated and/or contacted in the absence of serum during or during at least a portion of the incubation and/or contacting. In certain embodiments, the input composition is incubated and/or contacted in a serum-free medium during or during at least a portion of the incubation and/or contacting. In some embodiments, the serum-free medium is a defined and/or well-defined cell culture medium. In some embodiments, a serum-free medium is formulated to support the growth, proliferation, health, homeostasis of cells of a certain cell type (e.g., immune cells, T cells, and/or CD4+ and CD8+ T cells).

In some embodiments, the cell concentration of the input composition is from 1.0×105 cells/mL to 1.0×10¹⁰ titers. In some embodiments, the transduction can be achieved at a multiplicity of infection (MOI) of less than 100, for example, typically less than 60, 50, 40, 30, 20, 10, 5, 4, 3, 2, 1, or less.

In some embodiments, the method involves contacting or incubating, e.g., mixing, cells with viral particles. In some embodiments, the contacting is performed for 30 minutes to 72 hours, such as 30 minutes to 48 hours, 30 minutes to 24 hours, or 1 hour to 24 hours, such as at least 30 minutes, 1 hour, 2 hours, 6 hours, 12 hours, 24 hours, 36 hours or more.

In some embodiments, the contacting is performed in a solution. In some embodiments, the cells and viral particles are contacted in a volume of 0.5 mL to 500 mL, such as a volume of 0.5 mL-200 mL, 0.5 mL-100 mL, 0.5 mL-50 mL, 0.5 mL-10 mL, 0.5 mL-5 mL, 5 mL-500 mL, 5 mL-200 mL, 5 mL-100 mL, 5 mL-50 mL, 5 mL-10 mL, 10 mL-500 mL, 10 mL-200 mL, 10 mL-100 mL, 10 mL-50 mL, 50 mL-500 mL, 50 mL-200 mL, 50 mL-100 mL, 100 mL-500 mL, 100 mL-200 mL or 200 mL-500 mL.

In some embodiments, the contacting can be accomplished by centrifugation, e.g, spin-seeding (e.g., centrifugation-seeding). In some embodiments, the composition containing cells, viral particles, and reagents can be spun, typically at a relatively low force or speed, e.g., a speed lower than that used to pellet cells, e.g., 600 rpm to 1700 rpm (e.g., at least 600 rpm, 1000 rpm or 1500 rpm or 1700 rpm). In some embodiments, the spinning is performed with a force (e.g., relative centrifugal force) of 100 g to 3200 g (e.g., at least 100 g, 200 g, 300 g, 400 g, 500 g, 1000 g, 1500 g, 2000 g, 2500 g, 3000 g, or 3200 g), as measured, for example, at the inner or outer wall of a chamber or cavity. The term “relative centrifugal force” or RCF is generally understood as the force exerted on an object or substance (such as a cell, sample or pellet and/or a point of a rotating chamber or other container), relative to the earth's gravity at a specific point in a space as compared with the axis of rotation.

In some embodiments, the incubation of cells with viral vector particles results in or produces an output composition comprising the cells transduced with the viral vector particles.

In some embodiments, after further incubation, the process of preparing cells may further include washing or formulating the cells. Therefore, the formulation of such compositions may be included in the processing steps.

In some embodiments, the cells and compositions are administered to a subject in a form of a pharmaceutical composition or formulation (e.g., a composition comprising a cell or population of cells and a pharmaceutically acceptable carrier or excipient).

The term “pharmaceutical formulation” refers to a formulation that is in a form effective to maintain the biological activity of the active ingredient contained therein and is free of additional components that would have unacceptable toxicity to the subject to whom the formulation is administered.

In some embodiments, the pharmaceutical composition additionally comprises other pharmaceutically active agents or drugs, for example, chemotherapeutic agents, such as asparaginase, busulfan, carboplatin, cisplatin, daunorubicin, doxorubicin, fluorouracil, gemcitabine, hydroxyurea, methotrexate, paclitaxel, rituximab, vinblastine, vincristine, and the like. In some embodiments, the agent is administered in a salt form, e.g., a pharmaceutically acceptable salt. Suitable pharmaceutically acceptable acid addition salts include those derived from inorganic acids, such as hydrochloric, hydrobromic, phosphoric, metaphosphoric, nitric and sulfuric acids, and those derived from organic acids, such as tartaric acid, acetic acid, citric acid, malic acid, lactic acid, fumaric acid, benzoic acid, glycolic acid, gluconic acid, succinic acid, and arylsulfonic acid (e.g., p-toluenesulfonic acid).

“Pharmaceutically acceptable carrier” refers to ingredients in a pharmaceutical formulation other than the active ingredient that are not toxic to the subject. Pharmaceutically acceptable carriers include, but not limited to, buffers, excipients, stabilizers or preservatives.

In some aspects, the choice of vector depends in part on the particular cell and/or method of administration. Therefore, there are a variety of suitable formulations. For example, the pharmaceutical composition may contain a preservative. Suitable preservatives may include, for example, methylparaben, propylparaben, sodium benzoate, and benzalkonium chloride. In some aspects, a mixture of two or more preservatives is used. The preservatives or mixtures thereof are generally present in an amount of from about 0.0001% to about 2% by weight of the total composition. Pharmaceutically acceptable carriers are generally non-toxic to recipients at the used dosages and concentrations, including, but not limited to: buffers, such as phosphates, citrates, and other organic acids; antioxidants, including ascorbic acid and methionine; preservatives (such as octadecyldimethylbenzylammonium chloride; hexamethylammonium chloride; benzalkonium chloride ammonium; benzethonium chloride; phenol, butanol or benzyl alcohol; alkyl parabens such as methyl or propyl paraben; catechol; resorcinol; cyclohexyl alcohol; 3-pentanol; and m-cresol); low molecular weight (less than about 10 residues) polypeptides; proteins, such as serum albumin, gelatin, or immunoglobulins; hydrophilic polymers, such as polyvinylpyrrolidone; amino acids, such as glycine, glutamine, asparagine, histidine, arginine, or lysine; monosaccharides, disaccharides, and other carbohydrates, including glucose, mannose, or dextrin; chelating agents, such as EDTA; carbohydrates, such as sucrose, mannitol, trehalose, or sorbitol; salt-forming counterions, such as sodium; metal complexes (e.g., zinc-protein complexes); and/or nonionic surfactants, such as polyethylene glycol (PEG).

In some aspects, buffering agents are included in the composition. Suitable buffering agents include, for example, citric acid, sodium citrate, phosphoric acid, potassium phosphate, and various other acids and salts. In some aspects, a mixture of two or more buffering agents is used. The buffering agent or mixtures thereof are generally present in an amount of from about 0.001% to about 4% by weight of the total composition. Methods for preparing administrable pharmaceutical compositions are known.

Formulations can include aqueous solutions. The formulation or composition may also contain more than one active ingredient useful for a particular indication, disease, which can be treated with cells, preferably those having complementary activities to the cells, respective activities of which will not interact with each other to cause negative effects. Such active ingredients are present in suitable combinations in amounts effective for the intended purpose. Therefore, in some embodiments, the pharmaceutical composition further includes other pharmaceutically active agents or drugs, for example chemotherapeutic agents, such as asparaginase, busulfan, carboplatin, cisplatin, daunorubicin, doxorubicin, fluorouracil, gemcitabine, hydroxyurea, methotrexate, paclitaxel, rituximab, vinblastine and/or vincristine.

In some embodiments, the pharmaceutical composition comprises cells in an amount effective to treat or prevent the disease (e.g., a therapeutically effective amount or a prophylactically effective amount). In some embodiments, therapeutic or prophylactic efficacy is monitored by periodically evaluating the treated subject. The desired dosage can be delivered by administering a single bolus of cells, by administering multiple boluses of cells, or by administering continuous infusions of cells.

In some embodiments, the compositions are provided as sterile liquid formulations (e.g., isotonic aqueous solutions, suspensions, emulsions, dispersions, or viscous compositions, which, in some aspects, can be buffered to a selected pH). Liquid formulations are generally easier to be prepare than gels, other viscous compositions, and solid compositions. Liquid or viscous compositions can contain a carrier, which can be a solvent or dispersion medium containing, for example, water, saline, phosphate buffered saline, polyol (e.g., glycerol, propylene glycol, liquid polyethylene glycol), and suitable mixtures thereof.

Sterile injectable solutions can be prepared by incorporating the cells into a solvent.

Various additives may be added to enhance the stability and sterility of the composition, including antimicrobial preservatives, antioxidants, chelating agents and buffering agents.

### Therapeutic Methods and Compositions for Administration

In some aspects, the products of the methods are used in therapeutic methods, e.g., a therapeutic method, e.g., for administering cells and compositions to a subject in adoptive cell therapy. The use of such methods and cells treated and produced by the methods, as well as pharmaceutical compositions and formulations for use therein are also provided. The provided methods generally involve administering cells or compositions (e.g., output compositions and/or formulated compositions) to a subject.

In some embodiments, the cells express recombinant receptors (e.g., CARs) or other antigen receptors (e.g., transgenic TCRs). Such cells are typically administered to a subject suffering from a disease associated with the ligand specifically recognized by the receptor. In one embodiment, the cell expresses a recombinant or chimeric receptor (e.g., an antigen receptor, such as a CAR or TCR) that specifically binds to a disease-associated ligand or a ligand expressed by a cell or tissue thereof. For example, in some embodiments, the receptor is an antigen receptor and the ligand is an antigen specific to and/or associated with a disease. The administration generally results in the amelioration of one or more symptoms of the disease and/or treatment or prevention of the disease or symptoms thereof.

Diseases, disorders include tumors, including solid tumors, hematologic malignancies, and melanomas, and including localized and metastatic tumors; infectious diseases, such as infections with viruses or other pathogens, such as HIV, HCV, HBV, CMV, and parasitic diseases; and autoimmune and inflammatory diseases. In some embodiments, the disease is a tumor, cancer, malignancy, tumor, or other proliferative disease. Such diseases include, but not limited to, leukemia, lymphoma (e.g., chronic lymphocytic leukemia (CLL), ALL, non-Hodgkin's lymphoma, acute myeloid leukemia, multiple myeloma, refractory follicular lymphoma, cell lymphoma, indolent B-cell lymphoma, B-cell malignancies), colon cancer, lung cancer, liver cancer, breast cancer, prostate cancer, ovarian cancer, skin cancer, melanoma, bone and brain cancer, ovarian cancer, epithelial cancer, renal cell carcinoma, pancreatic cancer, Hodgkin lymphoma, cervical cancer, colorectal cancer, glioblastoma, neuroblastoma, Ewing's sarcoma, medulloblastoma, osteosarcoma, synovial sarcoma and/or skin tumor.

In some embodiments, such diseases include, but not limited to, leukemias, lymphomas, such as acute myeloid (or myeloid) leukemia (AML), chronic myeloid (or myeloid) leukemia (CML), acute lymphoblastic (or lymphoblastic) leukemia (ALL), chronic lymphocytic leukemia (CLL), hairy cell leukemia (HCL), small lymphocytic lymphoma (SLL), mantle cell lymphoma (MCL), marginal zone lymphoma, Burkitt lymphoma, Hodgkin lymphoma (HL), non-Hodgkin lymphoma (NHL), anaplastic large cell lymphoma (ALCL), follicular lymphoma, refractory follicular lymphoma, diffuse large B-cell lymphoma (DLBCL) and multiple myeloma (MM). In some embodiments, the disease is a B cell malignancy selected from acute lymphoblastic leukemia (ALL), adult ALL, chronic lymphoblastic leukemia (CLL), non-Hodgkin lymphoma (NHL)) and diffuse large B-cell lymphoma (DLBCL). In some embodiments, the disease is NHL, and the NHL is selected from the group consisting of aggressive NHL, diffuse large B-cell lymphoma (DLBCL), NOS (de novo and indolent transformed), primary mediastinal large B-cell lymphoma (PMBCL), T-cell/histiocytic-rich large B-cell lymphoma (TCHRBCL), Burkitt lymphoma, mantle cell lymphoma (MCL) and/or follicular lymphoma (FL), optionally, 3B grade follicular lymphoma (FL3B).

In some embodiments, the disease is an infectious disease, such as, but limited to, viral, retroviral, bacterial and protozoal infections, immunodeficiency, cytomegalovirus (CMV), Epstein-Barrvirus (EBV)), adenovirus, BK polyoma virus infections. In some embodiments, the disease is an autoimmune or inflammatory disease, such as arthritis (e.g., rheumatoid arthritis (RA)), type I diabetes, systemic lupus erythematosus (SLE), inflammatory bowel disease, psoriasis, scleroderma, autoimmune thyroid disease, Graves disease, Crohn disease, multiple sclerosis, asthma and/or transplant-related disease.

As used herein, “treating” refers to the complete or partial amelioration or alleviation of a disease, or a symptom, adverse effect or outcome or phenotype associated therewith. Desirable therapeutic effects include, but not limited to, prevention of the occurrence or recurrence of a disease, alleviation of symptoms, reduction of any direct or indirect pathological consequences of a disease, prevention of metastasis, reduction in the disease progression, amelioration or alleviation of the state of a disease, and remission or improvement of prognosis. The terms do not imply complete cure of a disease or complete elimination of any symptoms or effects on all symptoms or outcomes.

As used herein, “delaying the development of a disease” means delaying, retarding, slowing, retarding, stabilizing, inhibiting and/or delaying the development of a disease (e.g., a cancer). Such delay can be of varying lengths, depending on the medical history and/or the individual being treated. It will be apparent to a skilled person that a sufficient or significant delay may actually encompass prevention, since the individual will not develop the disease. For example, the development of advanced cancers, such as metastases, may be delayed.

As used herein, “prevention” includes the prevention of the occurrence or recurrence of a disease in a subject who may be susceptible to, but has not yet been diagnosed with, the disease. In some embodiments, provided cells and compositions are used to delay the development or delay the progression of a disease.

As used herein, “inhibiting” a function or activity refers to a reduction in the function or activity when compared with otherwise identical conditions other than the condition or parameter of interest, or with another condition. For example, cells that inhibit tumor growth reduce the tumor growth rate compared with the tumor growth rate in the absence of the cells.

Methods for administering cells for adoptive cell therapy are known and can be used with the provided methods and compositions.

The treated disease may be any disease in which the expression of an antigen is associated with and/or involved in the etiology of the disease condition, e.g., causes, exacerbates, or otherwise participates in the disease, disorder. Exemplary diseases and disorders can include diseases associated with malignancies or cellular transformations (e.g., cancer), autoimmune or inflammatory diseases, or infectious diseases, e.g., caused by bacteria, viruses, or other pathogens. Exemplary antigens are described above, including those associated with various diseases and disorders that can be treated. In a specific embodiment, the chimeric antigen receptor or transgenic TCR specifically binds to an antigen associated with the disease.

Cells and compositions can be administered using standard administration techniques, formulations and/or devices. Administration of cells can be autologous or allogeneic, e.g., allogeneic. For example, immune response cells or progenitor cells can be obtained from one subject and administered to the same subject or to a different compatible subject. Peripheral blood-derived immune response cells or a progeny thereof (e.g., derived in vivo, ex vivo, or in vitro) can be administered by local injection, including catheter, systemic, local, intravenous, or parenteral administration. When a therapeutic composition (e.g., a pharmaceutical composition containing genetically modified immune response cells) is administered, it is usually formulated in an injectable form of unit dose (solution, suspension, emulsion).

In some embodiments, cell therapy (e.g., adoptive cell therapy, such as adoptive T cell therapy) is performed by autologous transfer, and cells were isolated from a subject who will receive the cell therapy or from a sample derived from such a subject and/or prepared in other ways. Therefore, in some aspects, the cells are derived from a subject (e.g., a patient) in need of treatment, and after isolation and process, the cells are administered to the same subject.

The cells can be given in any appropriate way, such as infusion, injection, such as intravenous or subcutaneous injection, intraocular injection, epidemic injection, subconductal injection Injecting, intraulinal injection, anterior room injection, subcconjectval injection, subconjuntival injection, sub-Tenon injection, retrobulbar injection, peribulbar injection, or posterior juxtascleral deliver. In some embodiments, they are given through the gastrointestinal, intravior, and intra-nose route and give inside lesions (if local treatment is needed). Parenteral infusion includes intramuscular, intravenous, intracera, peritoneal, or subcutaneous administration. In some embodiments, the given dose is given by a single bolus administration of cells, multiple-bolus administration of cells, or continuous infusion of cells.

In adoptive cell therapy, the administration of a given “dose” of cells includes the administration of a given amount or number of cells in the form of a single composition and/or a single uninterrupted administration (for example, in the form of a single injection or a continuous infusion), and also includes the administration of a given amount or number of cells in the form of a divided dose or multiple compositions provided in a plurality of individual compositions or infusions within a specified period of time, for example, not more than 3 days. Therefore, in some cases, the dose is a single or continuous administration of a specified number of cells, given or started at a single time point. However, in some cases, the dose is given in the form of multiple injections or infusions over a period of not more than three days, such as once a day for three or two days or by multiple infusions over a period of one day.

Therefore, in some aspects, the dose of cells is administered as a single pharmaceutical composition. In some embodiments, the dose of cells is administered in a variety of compositions that collectively contain the dose of cells.

The term “divided dose” refers to divided dosages so that they are administered over a period of more than one day. Such type of administration is included in the present method and is considered as a single dose. In some embodiments, the divided dosages of cells are administered in a plurality of compositions that collectively contain the dose of cells over a period of not more than three days.

In some embodiments, the dose of cells may be administered in a plurality of compositions or solutions (e.g., first and second, optionally more), each containing the dose of some cells. In some aspects, a plurality of compositions, each containing a different cell population and/or cell subtype, are optionally administered separately or independently for a certain period of time. For example, the cell population or cell subtype may include CD8⁺ and CD4⁺ T cells, respectively, and/or may include CD8⁺ and CD4⁺ enriched populations, such as CD4⁺ and/or CD8⁺ T cells, respectively, each of which include cells genetically engineered to express recombinant receptors. In some embodiments, the administration of the dosage comprises administering a first composition comprising a dosage of CD8⁺ T cells or a dosage of CD4⁺ T cells, and administering a second composition comprising another dosage of CD4⁺ T cells and CD8⁺ T cells.

In some embodiments, the dosage or composition of cells comprises a defined or target ratio of CD4⁺ cells expressing the recombinant receptor to CD8⁺ cells expressing the recombinant receptor and/or CD4⁺ cells to CD8⁺ cells, the ratio optionally being about 1:1, or between about 1:3 and about 3:1, for example about 1:1. In some aspects, the administration of compositions or dosages of different cell populations (e.g., CD4⁺: CD8⁺ ratio or CAR+CD4+: CAR+CD8+ ratio, e.g., 1:1) with a target or desired ratio involves the administration of a cell composition containing one of the populations, and the subsequent administration of a separate cell composition containing another populations, wherein the administration is performed at or about the target or desired ratio. In some aspects, the administration of a dosage or composition of cells with a defined ratio results in improved expansion, persistence, and/or antitumor activity of T cell therapy.

In some embodiments, cells are administered in a desired dosage, which in some aspects includes a desired dosage or number of cells or one or more cell types and/or a desired ratio of cell types. Therefore, in some embodiments, the cell dosage is based on the total number of cells (or the number of cells per kg of body weight) and the ratio of the desired individual population or subtype, such as the ratio of CD4+ to CD8+. In some embodiments, the cell dosage is based on the total number of cells or individual cell types in the desired individual population (or the number of cells per kg of body weight). In some embodiments, the dosage is based on a combination of such characteristics, such as the desired total number of cells, the desired ratio, and the desired total number of cells in a single population.

In some embodiments, cells are administered at or within a tolerance range of the desired output ratio of a plurality of cell populations or subtypes, such as CD4+ and CD8+ cells or subtypes. In some aspects, the desired ratio may be a specific ratio or may be a series of ratios. For example, in some embodiments, the ratio of CD4+ to CD8+ cells is between 1:5 and 5:1, or between 1:3 and 3:1, such as between 2:1 and 1:5. In some aspects, the tolerance difference is about 1%, about 2%, about 3%, about 4%, about 5%, about 10%, about 15%, about 20%, about 25%, about 30%, about 35%, about 40%, about 45%, about 50% of the desired ratio, including any value between these ranges.

In a specific embodiment, the number and/or concentration of cells refers to the number of cells expressing the recombinant receptor (e.g., CAR). In other embodiments, the number and/or concentration of cells refers to the number or concentration of all administered cells, T cells or peripheral blood mononuclear cells (PBMCs).

In some aspects, the size of the dosage is determined based on one or more criteria, such as the subject's response to existing treatments such as chemotherapy, the subject's disease burden such as tumor burden, volume, size or degree, the degree or type of metastasis, stage, and/or the possibility or incidence of toxic results in the subject, such as CRS, macrophage activation syndrome, tumor lysis syndrome, neurotoxicity and/or host immune responses to the administered cells and/or recombinant receptors.

In some embodiments, a relatively low dosage of cells, such as a suboptimal dosage of cells or a dose lower than a therapeutically effective amount of cells, may be administered, which may lead to an enhancement (e.g., increase or expansion) in the number of engineered cells present in the subject when stimulated in vivo (e.g., by endogenous antigens or exogenous agents). In any such embodiments, the expansion and/or activation of cells may occur together with exposure to antigens in vivo, for example, the expansion of engineered cells in vivo in a subject after the cells are administered. In some embodiments, the range, extent, or magnitude of in vivo amplification can be expanded, enhanced, or enhanced by a variety of methods that can regulate (e.g., increase) the expansion, proliferation, survival, and/or efficacy of the administered cells (e.g., cells expressing recombinant receptors).

Once cells are administered to a subject (e.g., a person), the biological activities of the cell population are measured, in some aspects, by any of many known methods. Parameters to be evaluated include the specific binding of cells to antigens, which are evaluated in vivo, for example, by imaging, or in vitro, for example, by ELISA or flow cytometry. In certain embodiments, the ability of a cell to destroy a target cell can be measured using any suitable methods known in the art. In some embodiments, the biological activities of cells are measured by determining the expression and/or secretion of some cytokines, such as CD107a, IFNγ, IL-2 and TNF. In some aspects, bioactivities are measured by evaluating clinical outcomes, such as reduction of tumor burden. In some aspects, toxicity results, persistence and/or expansion of cells, and/or the presence or absence of a host immune response are evaluated.

### Composition and Formulation

In some embodiments, cells comprising cells engineered with recombinant antigen receptors such as CAR or TCR are provided in a composition or formulation such as a pharmaceutical composition or formulation. Such compositions may be used according to the provided method and/or together with the provided formulation or composition, for example, for preventing or treating diseases and disorders, or for detection, diagnosis and prognosis methods.

The term “pharmaceutical formulation” refers to a formulation in a form, in which the bioactivities of active ingredients contained therein can be effective and additional components having unacceptable toxicities to the subject to whom the formulation is administered are not contained.

“Pharmaceutically acceptable carriers” refer to non-toxic ingredients to the subject other than the active ingredients in the pharmaceutical preparation. Pharmaceutically acceptable carriers include but not limited to buffers, excipients, stabilizers or preservatives.

In some aspects, the selection of a carrier is determined in part by a specific cell or agent and/or by a method of administration. Therefore, there are many suitable formulations. For example, the pharmaceutical composition may contain a preservative. Suitable preservatives may include, for example, methyl p-hydroxybenzoate, propyl p-hydroxybenzoate, sodium benzoate and benzalkonium chloride. In some aspects, a mixture of two or more preservatives is used. Pharmaceutically acceptable carriers are generally non-toxic to a recipient at the used dosages and concentrations, including but not limited to: buffers, such as phosphate, citrate and other organic acids; antioxidants, including ascorbic acid and methionine; preservatives (such as octadecyl dimethyl benzyl ammonium chloride; hexamethylammonium chloride; benzalkonium chloride; benzalkonium chloride; phenol, butanol or benzyl alcohol; alkyl p-hydroxybenzoates, such as methyl p-hydroxybenzoate or propyl p-hydroxybenzoate; catechol; resorcinol; cyclohexanol; 3-pentanol; and m-cresol); low molecular weight (less than about 10 residues) polypeptide; proteins, such as serum albumin, gelatin or immunoglobulin; hydrophilic polymers, such as polyvinylpyrrolidone; amino acids, such as glycine, glutamine, asparagine, histidine, arginine or lysine; monosaccharides, disaccharides and other carbohydrates, including glucose, mannose or dextrin; chelating agents such as EDTA; sugars, such as sucrose, mannitol, trehalose or sorbitol; salt-forming counterions, such as sodium; metal complexes (e.g., zinc protein complexes); and/or a nonionic surfactant, such as polyethylene glycol (PEG).

In some aspects, a buffer is included in the composition. Suitable buffers include, for example, citric acid, sodium citrate, phosphoric acid, potassium phosphate, as well as other acids and salts. In some aspects, a mixture of two or more buffers is used. The buffer or mixture thereof generally exists in an amount from about 0.001% to about 4% by weight of the total composition. The method for preparing a pharmaceutical composition for administration is known.

The preparation or composition can also contain more than one active ingredient, which can be used to prevent or treat specific indications and diseases with cells or drugs, in which respective activities will not adversely affect each other. Such active ingredients are present in suitable combinations in amounts that are effective for the intended purpose. Therefore, in some embodiments, the pharmaceutical composition further comprises other pharmaceutical active agents or drugs, such as chemotherapeutic agents, such as asparaginase, busulfan, carboplatin, cisplatin, daunorubicin, doxorubicin, fluorouracil, gemcitabine, hydroxyurea, methotrexate, taxol, rituximab, vinblastine, vincristine, etc. In some embodiments, the agent or cell is administered in the form of a salt, such as a pharmaceutically acceptable salt. Suitable pharmaceutically acceptable acid addition salts include those derived from inorganic acids (such as hydrochloric acid, hydrobromic acid, phosphoric acid, metaphosphoric acid, nitric acid and sulfuric acid) and organic acids (such as tartaric acid, acetic acid, citric acid, malic acid, lactic acid, fumaric acid, benzoic acid, glycolic acid, gluconic acid, succinic acid and aryl sulfonic acid, such as p-toluenesulfonic acid).

In some embodiments, the pharmaceutical composition contains a drug or cell in an amount that is effective in treating or preventing a disease, such as a therapeutic effective amount or a preventive effective amount. In some embodiments, the efficacy of treatment or prevention is monitored by periodically evaluating the treated subjects. For repeated administration for several days or longer, the treatment is repeated until the inhibition of the desired disease symptoms occurs, depending on the disease. However, other dose regimens may be useful and can be determined. The required dosage may be delivered by single bolus administration of the composition, by multiple bolus administration of the composition, or by continuous infusion of the composition.

The drug or cell can be given by any suitable method, such as bolus infusion, injection, such as intravenous or subcutaneous injection, intraocular injection, periocular injection, subretinal injection, intravitreal injection, septal injection, subscleral injection, choroidal injection, anterior chamber injection, subconjunctival injection, subconjunctival injection, retrobulbar injection, peribulbar injection or post scleral delivery. In some embodiments, they are administered through parenteral, intrapulmonary, and intranasal administration, as well as intralesional administration. Parenteral infusion includes intramuscular, intravenous, arterial, intraperitoneal or subcutaneous administration. In some embodiments, a given dosage is given by a single bolus administration of cells or drugs. In some embodiments, a given dosage is given, for example, by multiple injections of cells or drugs or by continuous infusion of cells or drugs in a time period not exceeding 3 days.

For the prevention or treatment of a disease, an appropriate dosage may depend on the type of disease to be treated, the type of one or more drugs, the type of cells or recombinant receptors, the severity and course of the disease, whether the agents or cells are given for preventive or therapeutic purposes, the prior treatment, the clinical history of the subject and the reaction to the agents or cells, and the decision of the attending physician. In some embodiments, the composition is suitable for the administration to a subject at one time or in a series of treatments.

Cells or drugs may be administered using standard dosing techniques, preparations and/or equipments. Preparations and devices (such as syringes and vials) for storing and administering the composition are provided. With regard to cells, the administration can be autologous or allogeneic. For example, immune response cells are obtained from a subject and given to the same subject or different compatible subjects. Peripheral blood derived immune response cells or offsprings thereof can be administered via local injection, including catheter administration, systemic injection, local injection, intravenous injection or parenteral administration. When a therapeutic composition (such as a pharmaceutical compositions containing genetically modified immune response cells or drugs for treating or improving neurotoxic symptoms) are administered, it is usually formulated in an injectable form at a unit dosage.

In some embodiments, the given cells (such as cells engineered to express recombinant receptors) are modified to expand, enhance, or enhance the amplification, proliferation, survival, and/or efficacy of the given cells. In some embodiments, the given cells (for example, cells engineered to express recombinant receptors) are modified so that the amplification, proliferation, survival and/or efficacy of the engineered cells can be regulated and/or controlled, for example, by administration of a drug.

In some embodiments, the method includes an in vivo step of reducing, inhibiting, and/or minimizing the effects of inhibitors on the proliferation, expansion, and/or survival of engineered cells in vivo. In some embodiments, the method includes in vivo steps to promote, support, and/or enhance the proliferation, expansion, and/or survival of engineered cells in vivo.

In some embodiments, additional agents are small molecules, peptides, polypeptides, antibodies or antigen binding fragments thereof, antibody mimics, aptamers or nucleic acid molecules (such as siRNA), lipids, polysaccharides, or any combination thereof. In some embodiments, additional agents are inhibitors or activators of specific factors, molecules, receptors, functions, and/or enzymes. In some embodiments, additional agents are agonists or antagonists of specific factors, molecules, receptors, functions, and/or enzymes. In some embodiments, additional agents are analogs or derivatives of one or more factors and/or metabolites. In some embodiments, additional agents are proteins or peptides. In some embodiments, other agents are cells, such as engineered cells.

### Agents for Specific Amplification of Transgene

In some embodiments, the method includes, for example, administering a medicament other than a given cell (such as a cell engineered to express a recombinant receptor) in a combination therapy. In some embodiments, the agent specifically amplifies, enhances, or augments the amplification, proliferation, survival, and/or efficacy of engineered cells due to specific regulation of transgenes, such as transgenes encoding recombinant receptors. In some embodiments, the agent specifically targets transgenes, such as recombinant receptors. In some embodiments, the agent specifically binds, activates and/or enhances the activity of the recombinant receptor and/or other functions of all or part of recombinant molecules encoded by transgene. In some embodiments, the combination of the agent and the recombinant cell can enhance, enhance or expand the proliferation, amplification and/or survival of the given cell, for example, to enhance the in vivo amplification of the cell.

In some embodiments, exemplary methods or agents for the specific amplification of a transgene include endogenous antigen exposure, vaccination, anti idiotypic antibodies or antigen binding fragments thereof, and/or adjustable recombinant receptors. For example, in some embodiments, a method for the specific amplification of a transgene includes a vaccination method. In some embodiments, a method for the specific amplification of a transgene includes administering an anti idiotypic antibody. Antiidiotypic antibodies (including antigen-binding fragments thereof) specifically recognize, specifically target, and/or specifically bind to idiotopes of antibodies or antigen binding fragments thereof (such as the antigen-binding domain of recombinant receptors (such as chimeric antigen receptors (CARs)). The idiotopes is any single antigenic determinant or epitope within the variable part of an antibody. In some embodiments, the anti idiotypic antibody or an antigen-binding fragment thereof is an agonist and/or exhibits specific activity to stimulate the expression of specific antibodies by cells, and the specific antibodies include conjugates or recombinant receptors containing the antibody or antigen-binding fragment thereof.

In some embodiments, the method includes the regulation of expansion, proliferation, survival and/or activity of immune cells or immune functions (usually including the given engineered cells). In some embodiments, the method includes steps that are generally immuno-stimulatory or generally promote, augment, expand and/or enhance the expansion, proliferation, survival and/or activity of immune cells (including the given cells) in vivo (such as in a subject). In some embodiments, the agent may reduce, inhibit, and/or minimize the effects of an inhibitory factor on the proliferation, expansion, and/or survival of immune cells (such as cells given) in vivo.

In some embodiments, the method includes regulating the amplification of engineered cells, for example, by inhibiting negative regulators for the proliferation, amplification, and/or activation of the given cells (such as engineered immune cells). In the specific environment of the subject, the given cells expressing recombinant receptors may encounter an environment that suppresses or inhibits the growth, proliferation, expansion and/or survival of cells, such as an immunosuppressive environment. For example, an immunosuppressive environment may contain immunosuppressive cytokines, regulatory regulators, and co-inhibitory receptors.

In some embodiments, additional agents include immunomodulators, immunocheckpoint inhibitors, metabolic pathway modulators, adenosine pathway or adenosine receptor antagonists or agonists, and modulators of signaling pathways (such as kinase inhibitors).

In some embodiments, other agents are immunomodulators, such as an immunocheckpoint inhibitors. In some embodiments, other agents increase, enhance, or amplify the amplification and/or proliferation of the given cells, thereby increasing, enhancing, or amplifying the immune response by blocking immune checkpoint proteins (i.e., immune checkpoint inhibitors). In some embodiments, other agents are agents that enhance activities of engineered cells (such as recombinant receptor-expressing cells), and are molecules that inhibit immunosuppressive molecules or immune checkpoint molecules. Examples of the immunosuppressive molecule include PD-1, PD-L1, CTLA4, TEVI3, CEACAM (for example, CEACAM-1, CEACAM-3 and/or CEACAM-5), LAG3, VISTA, BTLA, TIGIT, LAIR1, CD160, 2B4 and TGFRβ.

In some embodiments, an immunocheckpoint inhibitor may be an antibody against an immunocheckpoint protein, such as an antibody against cytotoxic T lymphocyte antigen 4 (CTLA4 or CD152), programmed cell death protein 1 (PD-1), or programmed cell death protein 1 ligand 1 (PD-L1).

In some embodiments, the method includes contacting cells expressing recombinant receptors with an agent inhibiting inhibitory cell surface receptors (such as transforming growth factor β Receptor (TGF β R)). In some embodiments, the given cells (such as recombinant receptor-expressing cells) can be engineered to resist the effects of immunosuppressive cytokines that inhibit their effectors function. In some embodiments, other agents are anti TGF β antibodies or anti TGF β R antibodies.

In some embodiments, other agents regulate the metabolism, signal transduction, and/or transport of immunosuppressive factors, such as adenosine. In some embodiments, other agents are inhibitors of extracellular adenosine or adenosine receptors, or agents that reduce or decrease extracellular adenosine levels, such as agents that prevent the formation of extracellular adenosine, degrade extracellular adenosine, inactivate extracellular adenosine, and/or reduce extracellular adenosine. In some embodiments, other agents are adenosine receptor antagonists, such as A2a, A2b, and/or A3 receptors.

In some embodiments, other agents are adenosine receptor antagonists or agonists, such as antagonists or agonists of one or more of adenosine receptors A2a, A2b, A1 and A3.

In some embodiments, the method includes administering other immunostimulatory agents. In some embodiments, other agents can generally promote the proliferation, expansion, survival and/or efficacy of immune cells. In some embodiments, other agents may specifically promote the given cells, such as cells expressing recombinant receptor. In some embodiments, other agents are cytokines. In some embodiments, other agents are ligands.

In some embodiments, other agents are immunostimulatory ligands, such as CD40L. In some embodiments, other agents are cytokines, such as IL-2, IL-3, IL-6, IL-11, IL-7, IL-12, IL-15, IL-21, granulocyte macrophage colony stimulating factor (GM-CSF), α, β or γ Interferon (IFN) and erythropoietin (EPO).

In some aspects, the provided method may also include, for example, giving one or more lymphocyte clearance prior to or at the same time as the administration of cells (e.g., recombinant receptor-expressing cells). In some embodiments, the lymphocyte clearance therapy includes the administration of cyclophosphamide. In some embodiments, the lymphocyte clearance therapy includes the administration of fludarabine. In some embodiments, the lymphocyte clearance therapy is not given.

The pretreatment of a subject with immune clearance (e.g., lymphocyte clearance) therapy can improve effects of adoptive cell therapy (ACT). The pretreatment with lymphocyte scavengers (including a combination of cyclosporine and fludarabine) has effectively improved the efficacy of metastatic tumor infiltrating lymphocytes (TIL) in cell therapy, including improving the response and/or persistence of metastatic cells.

In some embodiments, the provided method also involves administering lymphocyte clearance therapy to a subject. In some embodiments, the method involves administering lymphocyte clearance therapy to a subject prior to administering a dosage of cells. In some embodiments, lymphocyte clearance therapy comprises a chemotherapeutic agent.

In some embodiments, the method includes administering a preconditioning agent, such as a lymphocyte scavenger or a chemotherapy agent, such as cyclophosphamide, fludarabine, or a combination thereof, to a subject before administering a dosage of cells. For example, the pretreatment agent can be given to a subject at least 2 days, such as at least 3, 4, 5, 6 or 7 days before the first or subsequent dosages. In some embodiments, the pretreatment agent is administered to a subject not more than 7 days, such as not more than 6, 5, 4, 3, or 2 days before a dosage of cells is administered.

In some embodiments, a subject is pretreated with cyclophosphamide at a dosage of or between about 20 mg/kg and 100 mg/kg, such as at or between about 40 mg/kg and 80 mg/kg.

In some embodiments, fludarabine may be administered in a single dosage or in multiple dosages, such as daily, every other day, or every three days. In some embodiments, fludarabine is given daily for 1-5 days, such as 3-5 days. In some cases, a subject is given fludarabine of about 30 mg/m2 every day for 3 days before starting the cell therapy. In some embodiments, cyclophosphamide is administered once daily for one or two days.

In an exemplary dosage regimen, before receiving the first dosage, a subject receives lymphocyte clearance preconditioning chemotherapy of cyclophosphamide and fludarabine (cy/flu), which is administered at least 2 days before the first dosage of CAR-expressing cells and usually no more than 7 days before the administration of cells. After preconditioning treatment, a subject is given the dosage of CAR-expressing T cells as described above.

In some embodiments, the administration of a preconditioning agent prior to the infusion of a dosage of cells improves the outcome of the treatment. For example, in some aspects, preconditioning improves the efficacy of dose therapy or increases the persistence of cells expressing recombinant receptors (such as CAR-expressing cells, such as CAR-expressing T cells) in a subject. In some embodiments, the pretreatment increases disease-free survival, such as the percentage of surviving subjects, and does not show minimal residual or molecularly detectable disease after a given period of time after the dosage of cells is given. In some embodiments, time to median disease-free survival is increased.

After the cells are given to a subject (such as a person), in some aspects, biological activities of the engineered cell population are measured by any one of many known methods. Parameters to be evaluated include the specific binding of engineered or natural T cells or other immune cells to antigens, which are evaluated in vivo, for example, by imaging, or in vitro, for example, by ELISA or flow cytometry. In some embodiments, biological activities of the cells can be measured by determining the expression and/or secretion of certain cytokines, such as CD107a, IFNγ, IL-2 and TNF. In some aspects, the biological activity is measured by evaluating clinical outcomes, such as reduced tumor burden or burden. In some aspects, the presence or absence of toxicity results, cell persistence and/or amplification and/or host immune responses can be evaluated.

In some embodiments, the administration of a preconditioning agent prior to the infusion of a dosage of cells improves the outcome of treatment (for example, by improving the efficacy of treatment with a dose), or increases the persistence of cells expressing recombinant receptors (for example, CAR-expressing cells, such as CAR expressing T cells) in a subject.

In some embodiments, cells are modified in any number of ways to increase their therapeutic or preventive efficacy and/or to regulate amplification, proliferation, survival, and/or efficacy thereof. In some embodiments, cells are modified so that, after being administered to a subject, their amplification, proliferation, survival, and/or function can be regulated (e.g., enhanced, strengthened, and/or expanded). In some embodiments, cells are modified so that the expression of a transgene and/or immunomodulatory factor can be regulated and/or controlled. In some embodiments, cells are modified to regulate the expression and/or activity of specific components of a recombinant receptor. In some embodiments, cells are modified to increase or decrease the expression of agents, such as nucleic acids, such as inhibitory nucleic acids. In some embodiments, cells are modified to express and/or secrete agents.

In some embodiments, engineered recombinant receptors (such as CAR) expressed by engineered cells can be directly or indirectly conjugated to a targeted portion through a connector.

In some embodiments, the method includes regulating given cells by contacting the cells with a medicament that reduces the expression of negative regulators of the given cells (such as engineered T cells expressing recombinant receptors) or is able to achieve the reduction in expression. Negative regulators of cells include any of those described herein, such as immunocheckpoint inhibitors, inhibitory receptors, and/or adenosine modulators. In some embodiments, agents that reduce the expression of negative regulators or can achieve the reduction in expression include agents that are or contain inhibitory nucleic acid molecules (such as nucleic acid molecules that are complement, target, inhibit and/or bind to genes or nucleic acids encoding negative regulators). In some embodiments, the agent is or comprises a complex, the complex comprises a ribonucleoprotein (RNP) complex, the ribonucleoprotein (RNP) complex comprises Cas9 (for example, Cas9 inactivated by enzyme in some cases) and a gRNA targeting a gene encoding a negative regulator.

In some of these embodiments, inhibitory nucleic acid molecules include RNA interfering agents. In some of these embodiments, inhibitory nucleic acids are or contain or encode small interfering RNA (siRNA), microRNA adapted shRNA, short hairpin RNA (shRNA), hairpin siRNA, precursor microRNA (pre miRNA), or microRNA (miRNA).

In some embodiments, engineered cells are subjected to gene modification or gene editing, which targets loci encoding genes involved in immune regulation, negative regulation of immune cells, and/or immunosuppression. In some embodiments, the gene editing results in an insertion or deletion at the targeted locus, or “knockout” of the targeted locus and elimination of the expression of the encoded protein. In some embodiments, the gene editing is realized by non homologous end joining (NHEJ) using CRISPR/Cas9 system. In some embodiments, one or more guide RNA (gRNA) molecules can be used with one or more Cas9 nucleases, Cas9 nicking enzymes, enzyme-inactivated Cas9 or variants thereof, or engineered zinc finger or TALE systems.

In some embodiments, the cells (e.g., recombinant receptor-expressing cells) are further modified to express and/or secrete additional agents that promote, enhance, enhance, and/or amplify the proliferation, expansion, survival, and/or efficacy of given cells. For example, recombinant receptor-expressing cells (such as CAR-expressing cells) can be further engineered to express and/or secrete additional agents that overcome immunosuppressive effects and/or enhance the expansion and/or function of T cells and recombinant receptors. In some embodiments, the cells may be engineered to express cytokines that promote the expansion of given cells. In some embodiments, such additional agents may be operatively connected to an inducible expression system, such as an inducible promoter.

In some embodiments, the given cells may be modified to express and/or secrete agents that inhibit immunosuppressive factors (such as any of those described herein) and/or stimulate immunostimulatory factors. In some embodiments, additional agents expressed by the given cells reduce or prevent immunosuppression of the cells in the tumor microenvironment. In some embodiments, additional agents encoded and/or secreted by the given cells may include any additional agents described herein.

In some embodiments, additional agents encoded by the given cells are soluble and secreted. In some embodiments, the additional agent is soluble scFv. In some embodiments, the additional agent is cytokine.

In some embodiments, the method includes modifying cells to allow for the regulated expression and/or activity of a recombinant receptors, such as CAR, thereby modulating signals through the recombinant receptors. In some embodiments, the regulated expression and/or activity is achieved by configuring the recombinant receptor to contain or be controlled by a specific regulatory element and/or system (such as any one described herein). In some embodiments, the administration of engineered cells to subjects and/or exposure to specific ligands may regulate the expression and/or activity of a recombinant receptor, such as a CAR. In some embodiments, the regulation of the expression and/or activity of a recombinant receptor is achieved by administering additional agents that can regulate the expression of the recombinant receptor, such as CAR. In some embodiments, the regulated expression of a recombinant receptor (such as CAR) is achieved by an adjustable transcription factor release system or by the administration of an additional agent that can induce changes in the conformation and/or polymerization of polypeptides (such as recombinant receptors). In some embodiments, the additional agent is a chemical inducer.

Unless otherwise defined, all of terms, symbols and other technical and scientific terms or designations used herein are intended to have the same meaning as commonly understood by a skilled person in the field to which the subject matter asked for the protection belongs. In some cases, terms with commonly understood meanings are defined herein for clarity and/or for ease of reference, and such definitions contained herein shall not be interpreted as representing substantive differences from those commonly understood in the art.

The term “about” or “approximate” refers to the usual error range of a corresponding value that is readily known to a skilled person. The reference herein to “about” a certain value or parameter includes (and describes) an embodiment of the value or parameter itself. For example, the description of “about X” includes the description of “X”. In some embodiments, “about X” or “approximate X” includes 50% X-150% X range, or 60% X-140% X range, or 70% X-130% X range, or 80% X-120% X range, or 90% X-110% X range, or 95%-105% X, or 97% X-103% X. For example, “about 4%” or “approximate 4%” includes 4%, or 2%-6%, or 2.4%-5.6%, or 2.8%-5.2%, or 3.2%-4.8%, or 3.6%-4.4%, or 3.88%-4.12%.

The term “subject” includes any living organisms, such as humans and other mammals. Mammals include but are not limited to human and non-human animals, including farm animals, sports animals, rodents and pets.

As used herein, when referring to one or more specific cell types or cell populations, “enrichment” means, for example, compared with the total number of cells in a composition or the volume of the composition or relative to other cell types, increasing the number or percentage of the cell type or population by, such as positive selection based on the markers expressed by the population or cells, or by negative selection based on the markers not present on the cell population or cells to be depleted. It is not necessary fot the term to completely remove other cells, cell types or populations from the composition, and it is not necessary for the enriched cells exist at or even close to 100% in the enriched composition.

As used herein, when a cell or cell population is described as being “positive” or “+” for a specific marker, it refers to the detectable presence of a specific marker (usually a surface marker) on or in a cell. When referring to a surface marker, it means that, in some embodiments, the presence of a surface expression is detected by flow cytometry for example, by staining with antibodies specifically bound to the markers and detecting the antibodies, wherein the staining is detectable at following level by flow cytometry, said level is basically higher than the staining detected by the same procedure with homotype matching control under the same conditions in other aspects, and/or said level is basically similar to the level of cells known to be positive for said marker, and/or said level is basically higher than the level of cells known to be negative for said marker.

As used herein, when a cell or cell group is described as being “negative” to a specific marker, it means that there is no detectable presence of the specific marker (usually a surface marker) on or in the cell. When referring to a surface marker, it means that, in some embodiments, for example, the absence of a surface expression is detected by flow cytometry, for example, by staining with antibodies specifically bound to the markers and detecting the antibodies, wherein the staining is not detected by flow cytometry at following levels: significantly higher than the level of staining detected by the same procedure with homotype matched controls under the same conditions in other aspects, and/or significantly lower than the level of cells known to be positive for the marker, and/or substantially similar as compared with cells known to be negative for the marker.

As used herein, for amino acid sequences (reference polypeptide sequences), “amino acid sequence identity percentage (%)” and “identity percentage” refers to the percentage of amino acid residues in a candidate sequence (e.g., Vpx or Vpr protein) that are the same as those in the reference polypeptide sequence, after the alignment of sequences and introduction of gaps when necessary to achieve the maximum sequence identity percentage and no conservative substitution is considered as part of sequence identity. The alignment used to determine the identity percentage of amino acid sequences can be achieved in many ways well known in the art, for example, using computer software available to the public, such as BLAST, BLAST-2, ALIGN or Megalign (DNASTAR) software. A skilled person can determine the appropriate parameters for the comparison sequence, including any algorithm required to achieve maximum alignment in the full length of the comparison sequence.

As used herein, a composition refers to any mixture of two or more products, substances or compounds (including cells). It can be a solution, suspension, liquid, powder, paste (aqueous, nonaqueous) or any combination thereof.

All publications (including patent literatures, scientific articles and databases) mentioned in this application are incorporated by reference in their entirety for all purposes, as if each individual publication is incorporated by reference. If the definitions set forth herein are contrary to or otherwise inconsistent with those set forth in patents, applications, public applications, and other publications incorporated herein by reference, the definitions set forth herein shall prevail over those incorporated herein by reference.

### ADVANTAGES OF THE INVENTION

The purpose of the invention is to provide a new method for preparing CAR-T cells. The preparation time for CAR-T cells by this method only takes about one day. Compared with the conventional preparation method of CAR T cells (about two weeks), the in vitro culture time will be greatly shortened, the memory phenotype of CAR-T cells will be better maintained, and the killing function of CAR T cells to tumors and their survival time in vivo will be enhanced.

The invention will be further described in combination with specific embodiments. It should be understood that these embodiments are only used to explain the invention and not to limit the scope of the invention. The following experimental methods without specific conditions specified in the embodiments are generally based on conventional conditions, such as J. Sam Brooke, et al., Guidelines for Molecular Cloning Experiments, Third Edition, Science Press, 2002, or according to the conditions recommended by the manufacturer.

### Example 1. Evaluation of Efficiency of Lentivirus Transduction

Samples rich in white blood cells were collected from a subject by leukocyte isolation, and white membrane layer was collected by Ficoll density gradient centrifugation to obtain peripheral blood mononuclear cells (PBMC) with high purity.

PBMC was washed and re-suspended in a buffer solution containing phosphate buffered saline (PBS), EDTA and human serum albumin. PBMCs were sorted to obtain CD4+CD8+ enriched T cell population.

PBMCs or enriched T cells were put into X-VIVO 15 medium (purchased from Lonza), and T cell activators, anti-CD3 and/or anti-CD28 bead reagents and lentivirus vector particles were added. The virus vector particles contained nucleic acids encoding chimeric antigen receptors (CAR-BCMA, amino acid sequence can be found in SEQ ID NO: 2, and nucleic acid sequence can be found in SEQ ID NO: 1). The lentivirus vector particles were added at the multiplicity of infection (MOI) of 3. After 24 hours, the culture solution was centrifuged and changed, washed with normal saline, and added to the freezing protection solution for freezing preservation. This preparation process is named as a new process.

| SEQ ID NO: 1 | |
|---|---|
| 1 | ATGGCCTTAC CAGTGACCGC CTTGCTCCTG CCGCTGGCCT TGCTGCTCCA |
| 51 | CGCCGCCAGG CCGGAGGTGC AATTGCTGGA GTCTGGGGGA GGCTTGGTAC |
| 101 | AGCCTGGGGG GTCCCTGAGA CTCTCCTGTG CAGCCTCCGG ATTCACCTTT |
| 151 | GGCGGTAATG CCATGTCCTG GGTCCGCCAG GCTCCAGGGA AGGGGCTGGA |
| 201 | GTGGGTCTCA GCAATTAGTG GTAATGGTGG TAGTACATTC TACGCAGACT |
| 251 | CCGTGAAGGG CCGGTTCACC ATCTCCAGAG ACAATTCCAA GAACACGCTG |
| 301 | TATCTGCAGA TGAACAGCCT GAGAGCCGAG GACACGGCCG TATATTACTG |
| 351 | TGCGAAAGTT CGTCCATTCT GGGGTACTTT CGACTACTGG GGCCAAGGAA |
| 401 | CCCTGGTCAC CGTCTCGAGT GGTGGAGGCG GTTCAGGCGG AGGTGGTTCT |
| 451 | GGCGGTGGCG GATCGGAAAT CGTGTTAACG CAGTCTCCAG GCACCCTGTC |
| 501 | TTTGTCTCCA GGGGAAAGAG CCACCCTCTC TTGCAGGGCC AGTCAGAGTG |
| 551 | TTAGCAGCAG CTACTTAGCC TGGTACCAGC AGAAACCTGG CCAGGCTCCC |
| 601 | AGGCTCCTCA TCTATGGAGC ATCCAGCAGG GCCACTGGCA TCCCAGACAG |
| 651 | GTTCAGTGGC AGTGGATCCG GGACAGACTT CACTCTCACC ATCAGCAGAC |
| 701 | TGGAGCCTGA AGATTTTGCA GTGTATTACT GTCAGCAGTA CTTCAACCCA |
| 751 | CCAGAATACA CGTTCGGCCA GGGGACCAAA GTGGAAATCA AACGTACCAC |
| 801 | GACGCCAGCG CCGCGACCAC CAACACCGGC GCCCACCATC GCGTCGCAGC |
| 851 | CCCTGTCCCT GCGCCCAGAG GCGTGCCGGC CAGCGGCGGG GGGCGCAGTG |
| 901 | CACACGAGGG GGCTGGACTT CGCCTGTGAT ATCTACATCT GGGCGCCCTT |
| 951 | GGCCGGGACT TGTGGGGTCC TTCTCCTGTC ACTGGTTATC ACCCTTTACT |
| 1001 | GCAAACGGGG CAGAAAGAAA CTCCTGTATA TATTCAAACA ACCATTTATG |
| 1051 | AGACCAGTAC AAACTACTCA AGAGGAAGAT GGCTGTAGCT GCCGATTTCC |
| 1101 | AGAAGAAGAA GAAGGAGGAT GTGAACTGAG AGTGAAGTTC AGCAGGAGCG |
| 1151 | CAGACGCCCC CGCGTACCAG CAGGGCCAGA ACCAGCTCTA TAACGAGCTC |
| 1201 | AATCTAGGAC GAAGAGAGGA GTACGATGTT TTGGACAAGA GACGTGGCCG |
| 1251 | GGACCCTGAG ATGGGGGGAA AGCCGCAGAG AAGGAAGAAC CCTCAGGAAG |
| 1301 | GCCTGTACAA TGAACTGCAG AAAGATAAGA TGGCGGAGGC CTACAGTGAG |
| 1351 | ATTGGGATGA AAGGCGAGCG CCGGAGGGGC AAGGGGCACG ATGGCCTTTA |
| 1401 | CCAGGGTCTC AGTACAGCCA CCAAGGACAC CTACGACGCC CTTCACATGC |
| 1451 | AGGCCCTGCC CCCTCGCTAG |

SEQ ID NO: 2
malpvtalllplalllhaarpevqllesggglvqpggslrlscaasgftfggnamswvrqapgkglewvsaisgng
gstfyadsvkgrftisrdnskntlylqmnslraedtavyycakvrpfwgtfdywgqgtlvtvssggggsggggsgg
ggseivltqspgtlslspgeratlscrasqsvsssylawyqqkpgqaprlliygassratgipdrfsgsgsgtdft
ltisrlepedfavyycqqyfnppeytfgqgtkveikrtttpaprpptpaptiasqplslrpeacrpaaggavhtrg
ldfacdiyiwaplagtcgvlllslvitlyckrgrkkllyifkqpfmrpvqttqeedgescrfpeeeeggcelrvkf
srsadapayqqgqnqlynelnlgrreeydvldkrrgrdpemggkpqrrknpqeglynelqkdkmaeayseigmkge
rrrgkghdglyqglstatkdtydalhmqalppr

As a conventional control, mononuclear cells obtained by leukocyte isolation or T cell populations obtained by enrichment are activated for 24 hours or 48 hours by adding bead reagents coupled with anti-CD3 and/or anti-CD28, and then lentivirus vectors are added at the multiplicity of infection (MOI) of 1-3, and the cells are continuously cultured for 7 days or more.

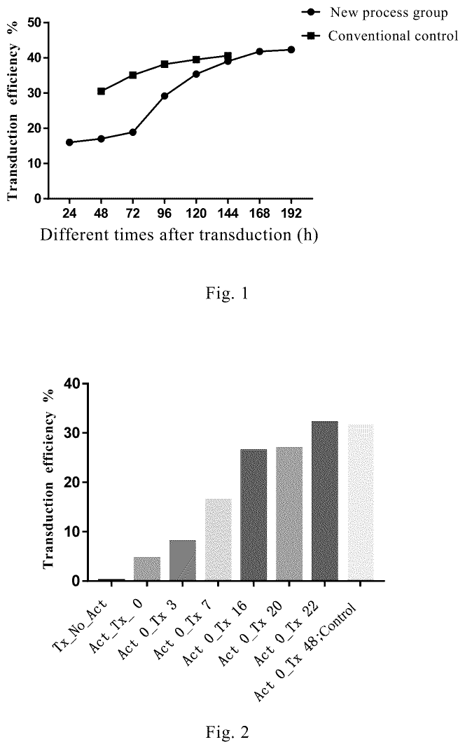

Flow cytometry was used to determine the efficiency of CAR transduction (or CAR T cell positive rate). CAR T cell positive rates at different time points after this method and conventional control transduction are shown in Table 1 and FIG. 1. As shown in Table 1 and FIG. 1, the transduction efficiency of CAR T cells harvested after 24 hours of culture in the new process is low, only 16.0%. After frozen and thawed, the CAR T cells were cultured in AIM-V medium supplemented with 2% AB serum and 300 IU/mL IL-2. It was found that the transduction efficiency detected in the early stage was significantly lower than that of the conventional control. However, with the extension of the culture time, the detected transduction efficiency continued to increase, and after 144 hours of culture, it tended to be stable, and was basically consistent with that of the conventional control. The results suggested that the transfection efficiency of the new process was very low because CARs were not fully expressed on the cell surface at harvest time. However, when they were re-infused into human body or cultured in vitro for a certain period of time, CARs could be fully expressed on the cell surface, and the positive rate reached the conventional control level.

TABLE 1
Changes in transduction efficiency at different time after transduction
by new process and conventional control (unit: %)

| | Different time after transduction (hour/h) | | | | | | | |
|---|---|---|---|---|---|---|---|---|
| Sample | 24 h | 48 h | 72 h | 96 h | 120 h | 144 h | 168 h | 192 h |
| new process | 16.0 | 17.0 | 18.9 | 29.2 | 35.4 | 39.0 | 41.8 | 42.3 |
| conventional | / | 30.5 | 35.1 | 38.2 | 39.5 | 40.6 | / | / |
| control | | | | | | | | |

In order to further verify the above results, CAR T cells targeting GPC3 were selected for studies on cell transduction. According to the above methods, PBMCs or enriched T cell populations, T cell activators and lentivirus vector particles were incubated together for 24 h for harvesting. Firstly, the transduction was performed at different multiplicity of infection (MOI), and CAR transduction efficiency was detected at 96 hours after transduction (results can be found in Table 2).

The new process was studied at the MOI of 3. The cells harvested after 24 hours of transduction were tested. The CAR transduction efficiency was almost undetectable (only 0.2%). The cells were continued to be cultured in AIM-V medium supplemented with 2% AB serum and 300 IU/mL IL-2. It was found that the detected transduction efficiency increased with the extension of in vitro culture time (as shown in Table 3).

TABLE 2
Transduction efficiency at different MOI (unit: %)

| | Transduction |
|---|---|
| MOI | efficiency at 96 h |
| 3 | 21.7 |
| 6 | 31.6 |
| 9 | 37.9 |
| 12 | 39.8 |

TABLE 3
Changes in transduction efficiency at different time
after transduction by the new process (unit: %)

| | Different time after transduction (h) | | | | | | | |
|---|---|---|---|---|---|---|---|---|
| Sample | 24 | 48 | 72 | 96 | 110 | 158 | 182 | 206 |
| New process | 0.2 | 5.9 | 11.4 | 18.5 | 31.0 | 39.2 | 43.1 | 48.7 |

The amino acid sequence of CAR of CAR T cells targeting GPC3 is shown in SEQ ID NO: 3.

Malpvtalllplalllhaarpevqlvqsgaevkkpgasvkvsckasgyt
fsdyemhwvrqapgqglewmgaihpgsgdtaynqrfkgrvtitadksts
taymelsslrsedtavyycarfysyaywgqgtlvtvsaggggggggsgg
ggsdivmtqtplslpvtpgepasiscrssqslvhsngntylqwylqkpg
qspqlliykvsnrfsgvpdrfsgsgsgtdftlkisrveaedvgvyycsq
siyvpytfgqgtkleikrtttpaprpptpaptiasqplslrpeacrpaa
ggavhtrgldfacdfwvlvvvggvlacysllvtvafiifwvrskrsrll
hsdymnmtprrpgptrkhyqpyapprdfaayrsrvkfsrsadapayqqg
qnqlynelnlgrreeydvldkrrgrdpemggkpqrrknpqeglynelqk
dkmaeayseigmkgerrrgkghdglyqglstatkdtydalhmqalppr

### Example 2: Evaluation on Transduction Efficiency of Primary T Cells after Activated and Cultured for 24-48 Hours

Samples rich in white blood cells were collected from a subjects by leukocyte isolation, and white membrane layer was collected by Ficoll density gradient centrifugation to obtain peripheral blood mononuclear cells (PBMCs) with high purity, which were added to the washed samples obtained from leukocyte isolation in the centrifuge chamber. Afterwards, standard methods were used to make cells pass from the transfer bag through an aseptic system of closed pipelines and a separation column in the presence of magnetic field to separate cells bound to CD4 specific reagent and/or CD8 specific reagent.

The enriched T cells were resuspended in the medium, incubated with CD3 and CD28 bead reagents for different times, and then transducted with lentivirus vector containing recombinant nucleic acid encoding GPC3 CAR. The amino acid sequence of GPC3 CAR is shown in SEQ ID NO: 3.

The process of activation and transduction was: about 2×10⁸ T cells were resuspended in the X-VIVO 15 medium with a total volume of 140 mL, inoculated into the culture bottle/bag, activated by adding anti-CD3 and CD28 bead reagents, and transducted by lentivirus vector at different activation times with MOI of 3. The total culture time for activation and transduction was 24 h, and harvested at the end. The culture solution was centrifuged and replaced with normal saline, and then resuspended in the frozen solution for cryopreservation.

Cells were cultured at 37° C. and 5% CO₂. The incubation time for activation and transduction of different schemes were as follows:

Scheme 1 (Tx_No_Act): no activator was added for activation, and cells were directly transduced, and harvest after 24 hours of culture.

Scheme 2 (Act_Tx_0): cells were activated and transduced simultaneously, and harvested after 24 hours of culture.

Scheme 3 (Act 0_Tx 3): cells were activated for 3 hrs, then transduced for 21 hours, and harvested after 24 hours of total culture time.

Scheme 4 (Act 0_Tx 7): cells were activated for 7 hrs, then transduced for 17 hours, and harvested after 24 hours of total culture time.

Scheme 5 (Act 0_Tx 16): cells were activated for 16 hrs, then transduced for 8 hours, and harvested after 24 hours of total culture time.

Scheme 6 (Act 0_Tx 20): cells were activated for 20 hrs, then transduced for 4 hours, and harvested after 24 hours of total culture time.

Scheme 7 (Act 0_Tx 22): cells were activated for 22 hrs, then transduced for 2 hours, and harvested after 24 hours of total culture time.

Scheme 8 (Act 0_Tx 48; control): a conventional process was used for control cells, which were activated for 48 h, and the lentivirus vector was added for transduction for 24 hs at MOI of 1.5. The free vector was removed by centrifugation, and the cells were further expanded and harvested on Day 8.

Cells harvested from Scheme 1 to Scheme 7 were inoculated into the culture medium after cryopreserved and thawed, and then cultured for 144 hr for testing the transduction efficiency, and the results can be found in FIG. 2. As shown in FIG. 2, compared with T cells that were activated and transduced at the same time, the surface expression of CAR was almost not detected in T cells that were not activated but transduced directly, indicating that T cells not activated are difficult to be directly transduced by lentivirus vector. In addition, under the same conditions of activation, the later the lentivirus vector was added, the higher the transduction efficiency was, although the actual duration of transduction decreased. Such results show that the activation state of T cells determines the difficulty of cells being transfected by lentivirus. Within a certain range, the longer the activation time is, the easier the cells are to be transfected. When the cells reach the easy transfection state, lentivirus can quickly enter the cells, which may be less than 2 hours, and the level of transduction efficiency is basically consistent with the conventional control level.

CAR T cells targeting CD19 (CD19 CAR) were prepared. The amino acid sequence of CAR is shown in SEQ ID NO: 4.

SEQ ID NO: 4:
Malpvtalllplalllhaarpqvqlvqsgaevkkpgssvkvsckasgyt
fssywmnwvrqapgqglewmgqiwpgdgdtnyngkfkgrvtitadests
taymelsslrsedtavyycarretttvgryyyamdywgqgttvtvssgg
ggsggggsggggsdivltqspaslavspgqratitckasqsvdydgdsy
lnwyqqkpgqppklliydasnlvsgvparfsgsgsgtdftltinpvean
dtanyycqqstedpwtfgqgtkveikrtttpaprpptpaptiasqplsl
rpeacrpaaggavhtrgldfacdiyiwaplagtcgvlllslvitlyckr
grkkllyifkqpfmrpvqttqeedgcscrfpeeeeggcelrvkfsrsad
apaykqgqnqlynelnlgrreeydvldkrrgrdpemggkprrknpqegl
ynelqkdkmaeayseigmkgerrrgkghdglyqglstatkdtydalhmq
alppr

The preparation process of CD19 CAR was activated and transduced according to the following scheme. Cells were cultured at 37° C. and 5% CO₂. The MOI for Scheme 1 to Scheme 5 was 3:

Scheme 1 (Act_Tx_0): cells were activated and transduced simultaneously, and harvested after 24 hours of culture.

Scheme 2 (Act 0_Tx 16): cells were activated for 16 hrs, then transduced for 8 hours, and harvested after 24 hours of total culture time.

Scheme 3 (Act 0_Tx 20): cells were activated for 20 hrs, then transduced for 4 hours, and harvested after 24 hours of total culture time.

Scheme 4 (Act 0_Tx 22): cells were activated for 22 hrs, then transduced for 2 hours, and harvested after 24 hours of total culture time.

Scheme 5 (Act 0_Tx 23): cells were activated for 23 hrs, then transduced for 1 hours, and harvested after 24 hours of total culture time.

Scheme 6 (Act 0_Tx 48; control): a conventional process was used for control cells, which were activated for 48 h, and the lentivirus vector was added for transduction for 24 hs at MOI of 1.5. The free vector was removed by centrifugation, and the cells were further expanded and harvested on Day 8.

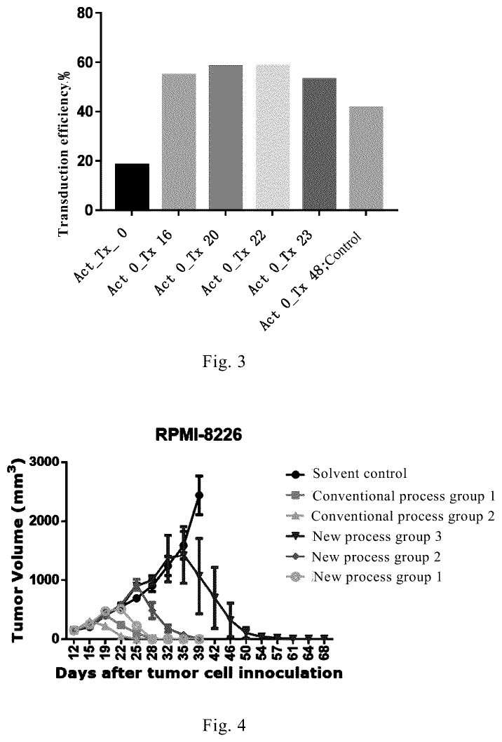

Cells harvested from Scheme 1 to Scheme 5 were inoculated into the culture medium after cryopreserved and thawed, and then cultured for 144 hr for testing the transduction efficiency, and the results can be found in FIG. 3. In addition, under a condition that cells were activated for 16 hours to 23 hours, then lentivirus vector was added for transduction and the total culture time was 24 hours, there is no significant difference in T cell transduction efficiency, which was slightly higher than that of conventional control, indicating that after T cells were activated to a certain state, the shortest time for lentivirus to enter the cells was only 1 hour.

### Example 3: Evaluation of Anti-Tumor Effects In Vivo

For evaluating anti-tumor activities of CAR T cells prepared by the new process, CAR T cells prepared by the conventional process (activated for 48 hours, then transduced, and cultured in vitro for more than 7 days after being transduced) were used as controls to compare anti-tumor effects of CAR-BCMA T cells of different dosages prepared by the new process in tumor-bearing mice. CAR-BCMA T cells prepared by conventional process were collected in D7 (culture for 7 days after transduction, defined as conventional process 1) and D11 (culture for 11 days after transduction, defined as conventional process 2) respectively. The activation and transduction of CAR-BCMA T cells prepared by the new process were conducted at the same time, and were transduced at MOI of 3. Samples were collected and frozen after 24 hours of culture. Relevant phenotypes were shown in Table 4 and the positive rate (for the new process, cells were cultured for 168 hours after cryopreservation and thawed) were shown in Table 5.

TABLE 4
Proportion of various types of T cells

| PBMC/CAR T cells | Th % | CTL % | TN % | TSCM % | TCM % |
|---|---|---|---|---|---|
| PBMCs for preparation | 31.3 | 40.2 | 25.4 | 5.3 | 6.8 |
| Conventional process 1 | 50.5 | 41.8 | 0.0 | 12.3 | 21.3 |
| Conventional process 2 | 35.7 | 60.6 | 0.0 | 3.1 | 15.2 |
| New process | 38.0 | 33.7 | 0.0 | 17.7 | 5.0 |

TABLE 5
Positive rate of CAR T cells for animal experiment

| Experiment groups | Positive rate (%) |
|---|---|
| Conventional process 1 | 35.1 |
| Conventional process 2 | 38.1 |
| New process | 34.9 |

Phenotypic results showed that compared with PBMCs before preparation, for the phenotype of BCMA-CAR T cells obtained by new process, the proportion of TN (CD3⁺CD95⁻CD62L⁺CD45RA⁺CCR7⁺CD45RO⁻) decreased from 25.4% to undetectable, and the proportion of TSCM (CD3⁺CD95⁺CD62L⁺CD45RA⁺CCR7⁺CD45RO⁻) increased from 5.3% to 17.7%, suggesting that Naive T cells (TN) transformed to TSCM after 24 hours of activation. The proportion of TSCM cultured to D7 and D11 by conventional process decreased significantly with culture time, which was 12.3% and 3.1% respectively, suggesting that TSCM would transform to Tcm with the extension of culture time. Therefore, for reducing the terminal differentiation of CAR T cells as much as possible and maintaining the effector function, the culture time for CAR T cells should be shortened as much as possible, and the new process is to shorten the culture time to 1-2 days. In addition, the positive rate results showed that the positive rate of CAR T cells prepared by the new process could reach the level of conventional CAR T cells.

CART cells prepared by the above new process were used to evaluate their anti-tumor efficacy in NPG mice bearing subcutaneously transplanted tumors of human multiple myeloma cells RPMI-8226, and their survival in the peripheral blood of mice. The day of tumor cell inoculation was recorded as DO, and the specific dosage and experiment design were shown in Table 6.

TABLE 6
Animal Experiment Dosage Design

| | | Dosage of CAR | |
|---|---|---|---|
| Experiment | | positive T cells | |
| category | Group | (×10\hat{ }6/mouse) | number |
| Negative control | Solvent group | 0 | 5 |
| Conventional | Conventional process 1 | 5 | 5 |
| process control | (collected on D 7) | | |
| | Conventional process 2 | 5 | 5 |
| | (collected on D 11) | | |
| New process | New process 3 | 0.2 | 5 |
| (cells were | New process 2 | 0.5 | 5 |
| cultured for 24 | New process 1 | 1.5 | 5 |
| hr and collected) | | | |

After tumor inoculation, CAR T cells were infused on D12. On Day 39 after tumor inoculation, the tumor volume in the solvent control group exceeded 2000 mm³. Compared with the solvent control group, the tumor volume, transplanted tumor and tumor regression in each group were listed as follows:
a) in conventional process group 1, the tumor volume inhibition rate was 100%, and all of the tumors of 5 mice regressed.
b) in conventional process group 2, the tumor volume inhibition rate was 100%, and all of the tumors of 5 mice regressed.
c) in new process group 1, the tumor volume inhibition rate was 100%, and all of the tumors of 5 mice regressed.
d) in new process group 2, the tumor volume inhibition rate was 100%, and all of the tumors of 5 mice regressed.
e) in new process group 3, the tumor volume inhibition rate was 56.05%, and none of the tumors of the mice regressed.

The tumor volumes of mice in the above groups and the changes in the mice weight with time are shown in FIG. 4. The onset time of new process group 1 was slightly later than that of conventional process group 1 and conventional process group 2, and the onset times of new process group 2 and new process group 3 were even later. There was a dosage correlation between the onset times and different dosages of CART in the new process groups. For the final efficacy, the complete tumor clearance was achieved in both of new process group 1 and new process group 2 within the observation period (Day 39 after tumor inoculation). The tumor volume of new process group 3 was also observed as being nearly stable from D32 to D35, and began to decrease until D39. Considering that the tumor volume of the solvent control group had reached the humanitarian end point according to the requirements of animal welfare, the animals in new process group 3 were retained to continue to observe the continuous efficacy. As expected, the tumor volume of mice in new process group 3 continued to decrease in the subsequent observation time. On Day 68 after tumor inoculation, the tumor volume inhibition rate of this dosage group was 100%, and almost all of the tumors in five mice regressed, showing encouraging anti-tumor effects. For the toxicity, the weight of mice did not change much during the experiment, except for the influence from tumors, suggesting that CAR T cells had no obvious toxic and side effects on mice.

Based on the huge anti-tumor potential of CAR-T cells prepared by the new process in the above animal experiments, it is speculated that when the cell infusion dose is reduced to 1/25 of the amount of cells used by the conventional process, and the tumor volume is increased to more than 1000 mm³, the tumor cells can still be rapidly cleared, suggesting that the new process has obvious advantages over the conventional process.

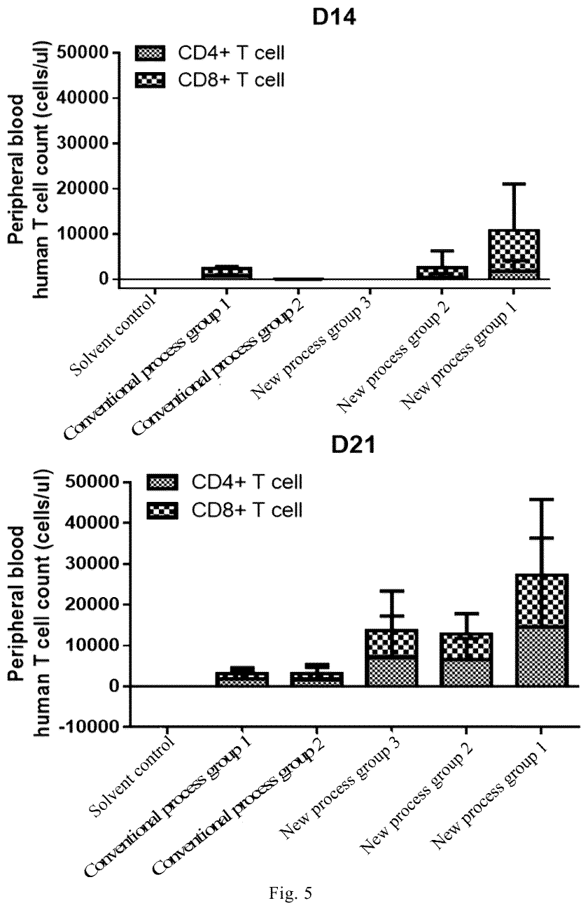

In addition, the survival of human T cells in the peripheral blood of mice in each group was detected on D14 and D21 after the infusion of CAR T cells, and the related results are shown in FIG. 5. Results showed that on Day 21, the mean number of CD3+ T cells in peripheral blood of the new process group with different dosages was significantly higher than that of the conventional process group, and the number of CD3+ T cells was increased compared with that on Day 14 after the CAR T were injected, suggesting that the proliferation of CAR T cells prepared by the new process was significantly better than that of the conventional process group in vivo. In addition, in the new process group 3, drugs were administered from Day 12 after tumor inoculation, tumors were almost eliminated on Day 68 day after tumor inoculation, and the process lasted for 56 days, which also indicated that CAR-T cells prepared by the new process had a stronger survival persistence in vivo.

### Example 4. Evaluation of Different T Cell Activators and Concentrations on Lentivirus Transduction

Samples rich in white blood cells were collected from subjects by leukocyte isolation, and white membrane layer was collected by Ficoll density gradient centrifugation to obtain peripheral blood mononuclear cells (PBMCs) with higher purity.

PBMCs were washed and re-suspended in a buffer, which were sorted based on immune affinity. The buffer contains phosphate buffered saline (PBS), EDTA and human serum albumin. For T cell sorting based on immune affinity, the washed cells in the sorting buffer were incubated with the bead reagent coupled to the monoclonal antibody at room temperature for 30 minutes, and the magnetic separation column was used for sorting.

The enriched T cells were resuspended in X-VIVO15 medium, and different T cell activators were added for activation. Lentivirus vectors containing nucleic acid encoding the chimeric antigen receptor (CAR-CD19) were also at MOI of 3. After 24 hrs of culture, the mediun was replaced with AIM-V medium supplemented with 2% AB serum and 300 IU/mL IL-2, the culture time was extended to 144 hrs, and the transduction efficiency at this time was used as the measurement basis for evaluating the activation conditions.

CD19-CAR T cells were prepared after being activated for 22 hours according to following reagent conditions and concentrations and then transduced for 2 hours. The MOI of transducing schemes 1 to 4 was 3, and the culture conditions were 37° C. and 5% CO₂.

Scheme 1: The cells were activated by bead reagent coupled with anti-CD3 and anti-CD28.

Scheme 2: Anti CD3 antibody at a final concentration of 500 ng/mL was used for cell activation.

Scheme 3: Anti CD3 antibody at a final concentration of 1000 ng/mL was used for cell activation.

Scheme 4: Anti CD3 antibody at a final concentration of 2000 ng/mL was used for cell activation.

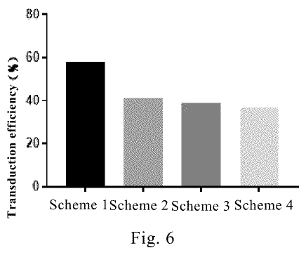

Cells from Scheme 1 to Scheme 4 were centrifuged for 2 hours after transduction, and then cultured to 144 hrs to test the transduction efficiency. The results are shown in Table 7 and FIG. 6. CAR T cells can be effectively prepared with the above two T cell activators and under three different concentrations.

TABLE 7
Transduction efficiency (unit: %)

| | Transduction |
|---|---|
| Conditions | efficiency |
| Scheme 1 | 58.1 |
| Scheme 2 | 41.1 |
| Scheme 3 | 38.7 |
| Scheme 4 | 36.8 |

All the documents mentioned in the present invention are cited as references in the application, just as each document is cited separately as a reference. In addition, it should be understood that after reading the above teachings of the present invention, a skilled person can make various changes or modifications to the present invention, and these equivalent forms also fall within the scope of the claims attached to the application.

1. A method for transducing cells with a viral vector, the method comprising:
Step (1), co-incubating an input composition comprising cells to be transduced, a stimulator for the cells to be transduced, and viral vector particles carrying a recombinant nucleic acid for an incubation time of no more than 72 hours,
Step (2), harvesting and obtaining an output composition comprising cells transduced with the recombinant nucleic acid;
preferably, the incubation time is 1 hour to 72 hours;
more preferably, the incubation time is 2 hours-48 hours;
more preferably, the incubation time is 2 hours-36 hours;
more preferably, the incubation time is 12 hours-36 hours;
more preferably, the incubation time is 12 hours-24 hours;
more preferably, the incubation time is 15 hours to 24 hours.

2. A method for transducing cells with a viral vector, the method comprising:
Step (1), incubating an input composition comprising cells to be transduced and a stimulator for the cells to be transduced for an incubation time of no more than 72 h,
step (2), adding and incubating viral vector particles carrying a recombinant nucleic acid for an incubation time of not more than 24 hours,
Step (3), harvesting and obtaining an output composition comprising cells transduced with the recombinant nucleic acid;
preferably, the total incubation time of steps (1) and (2) is no more than 72 hours.

3. The method of claim 2, wherein the total incubation time of steps (1) and (2) is no more than 60 hours, or no more than 48 hours, or no more than 32 hours, or no more than 24 hours.

4. The method of claim 2, wherein the incubation time of step (1) is 2-72 hours;
preferably, the incubation time in step (1) is 2-71 hours;
more preferably, the incubation time in step (1) is 2-48 hours;
more preferably, the incubation time in step (1) is 2-32 hours;
more preferably, the incubation time in step (1) is 2-28 hours;
more preferably, the incubation time in step (1) is 3-24 hours;
more preferably, the incubation time in step (1) is 5-24 hours;
more preferably, the incubation time in step (1) is 7-24 hours;
more preferably, the incubation time in step (1) is 7-23 hours;
more preferably, the incubation time in step (1) is 10-23 hours;
more preferably, the incubation time in step (1) is 15-23 hours;
more preferably, the incubation time in step (1) is 15-22 hours.

5. The method of claim 2, wherein the incubation time in step (2) is 30 mins-24 hours;
preferably, the incubation time in step (2) is 30 mins-21 hours;
preferably, the incubation time in step (2) is 30 mins-17 hours;
preferably, the incubation time in step (2) is 30 mins-12 hours;
preferably, the incubation time in step (2) is 30 mins-10 hours;
preferably, the incubation time in step (2) is 30 mins-8 hours;
preferably, the incubation time in step (2) is 1 hour-8 hours;
preferably, the incubation time in step (2) is 1 hour-4 hours;
more preferably, the incubation time in step (2) is 1 hours-3 hours.

6. The method of claim 2, wherein the input composition is obtained from peripheral blood, cord blood, bone marrow and/or induced pluripotent stem cells; preferably, the input composition is a leukopheresis sample; and preferably, the input composition is enriched or isolated CD3+ T cells, enriched or isolated CD4+ T cells or enriched or isolated CD8+ T cells or a combination thereof; or the number of cells to be transduced in the input composition is not higher than 1*10¹⁰;
preferably, the number of cells to be transduced in the input composition is not less than 1*10⁵;
and more preferably, the number of cells to be transduced in the input composition is not less than 1*10⁶.

7. The method of claim 2, wherein the viral vector particle is derived from a retroviral vector; and preferably, the viral vector particle is a lentiviral vector; preferably the multiplicity of infection of the viral vector particles is not higher than 20; preferably, the multiplicity of infection is 0.5-20; more preferably, the multiplicity of infection is 1.5-20; more preferably, the multiplicity of infection is 3-20; and more preferably, the multiplicity of infection is 3-12.

8-9. (canceled)

10. The method of claim 2, wherein the recombinant nucleic acid can encode a receptor that recognizes a specific target antigen; and preferably, the receptor that recognizes a specific target antigen is T cell receptor (TCR), chimeric antigen receptor (CAR), chimeric T cell receptor, or T cell antigen coupler (TAC); preferably, the specific target antigen is a disease-associated antigen or a universal tag;
preferably, the disease is a cancer, autoimmune disease, or infectious disease;
preferably, the cancer is a hematological tumor; and more preferably, the hematological tumor is a leukemia, myeloma, lymphoma and/or a combination thereof;
preferably, the specific target antigen is a tumor-associated antigen;
preferably, the tumor-associated antigen is selected from: B cell maturation antigen (BCMA), carbonic anhydrase 9 (CAIX), EGFR, Her2/neu (receptor tyrosine kinase erbB2), CD19, CD20, CD22, mesothelin, CEA, CD23, CD24, CD30, CD33, CD38, CD44, EGFR, Epiglin 2 (EPG-2), Epiglin 40 (EPG-40), EPHa2, erb-B2, erb-B3, erb-B4, erbB dimer, EGFR vIII, folic acid Binding protein (FBP), FCRL5, FCRH5, fetal acetylcholine receptor, GD2, GD3, HMW-MAA, IL-22R-α, IL-13R-α2, kinase insertion domain receptor (kdr), L1 cell adhesion molecule (L1-CAM), melanoma-associated antigen (MAGE), TAG72, B7-H6, IL-13 receptor alpha 2 (IL-13Ra2), CA9, GD3, HMW-MAA, CD171, G250/CAIX, HLA-AI MAGEA1, HLA-A2, PSCA, folate receptor, CD44v6, CD44v7/8, avb6 integrin, 8H9, NCAM, VEGF receptor, 5T4, fetal AchR, NKG2D ligand, CD44v6, mesothelin, mucin 1 (MUC1), MUC16, PSCA, NKG2D, NY-ESO-1, MART-1, gp100, carcinoembryonic antigen, G protein-coupled receptor 5D (GPCR5D), ROR1, TAG72, VEGF-R2, carcinoembryonic antigen (CEA), prostate specific antigen, PSMA, ephrin B2, CD123, c-Met, GD-2, O-acetylated GD2 (OGD2), CE7, Wilms tumor 1 (WT-1), Cyclin, CCL-1, CD138, Claudin18.2, GPC3.

11-12. (canceled)

13. The method of claim 2, wherein the stimulator for the cells to be transduced is capable of activating one or more intracellular signaling domains of one or more components of a TCR complex or one or more intracellular signaling domains of one or more costimulatory molecules;
and preferably, the stimulator for the cells to be transduced comprises (i) a primary agent that specifically binds to a member of the TCR complex, optionally CD3, and (ii) a secondary agent that specifically binds to the T cell costimulatory molecule, optionally wherein the costimulatory molecule is selected from CD28, CD137(4-1-BB), OX40 or ICOS;
preferably, the stimulator for the cells to be transduced comprises a CD3 binding molecule, a CD28 binding molecule, recombinant IL-2, recombinant IL-15, recombinant IL-7, recombinant IL-21 or a combination thereof;
and preferably, the stimulator for the cells to be transduced comprises an anti-CD3 antibody and/or an anti-CD28 antibody.

14. The method of claim 2, wherein the cells to be transduced are immune effector cells;
preferably, the cells to be transduced are T cells, NK cells, NKT cells, dendritic cells, macrophages, CIK cells, and stem cell-derived immune effector cells or a combination thereof;
and more preferably, the cells to be transduced are T cells.

15. (canceled)

16. The method of claim 2, wherein the stimulator for the cells to be transduced can be removed by centrifugation prior to harvesting.

17. The method of claim 2, wherein the stimulator for the cells to be transduced is a free molecule; or
the stimulator for the cells to be transduced is immobilized on a solid support;
preferably, the solid support is a polymer matrix material;
and more preferably, the polymer matrix material is a degradable polymer nanomatrix or bead reagent.

18. (canceled)

19. The method of claim 17, wherein the bead reagent is a magnetic bead or microbead.

20. The method of claim 1, wherein the content of cells transduced with the recombinant nucleic acid in the output composition is not less than 30%, or not less than 40%, or not less than 50%, or not less than 60%, or not less than 70%, or not less than 80%; or
the content of cells transduced with the recombinant nucleic acid in the output composition is not higher than 50%; preferably, not higher than 40%, more preferably, not higher than 38%; more preferably, not higher than 35%; more preferably, not higher than 30%.

21. (canceled)

22. The method of claim 20, wherein:
(1) compared with the content of naive cells in the cells to be transduced, the content of naive cells in the cells transduced with the recombinant nucleic acid is reduced;
preferably, the content of naive cells is reduced to less than 10%;
and more preferably, the content of naive cells is reduced to less than 5%;
(2) compared with the content of memory cells in the cells to be transduced, the content of memory cells in the cells transduced with the recombinant nucleic acid is increased;
preferably, the memory cells are memory stem cells;
and more preferably, the memory stem cells are TSCMs;
(3) the content of memory stem cells in the cells transduced with recombinant nucleic acid is about 2 times or more the content of memory stem cells in the cells to be transduced, Preferably, the content of memory stem cells in the cells transduced with recombinant nucleic acid is about 3 times or more the content of memory stem cells in the cells to be transduced; or
(4) the cells transduced with the recombinant nucleic acid contain undifferentiated cells.

23-25. (canceled)

26. The method of claim 2, wherein the input composition comprises recombinant IL-2, optionally recombinant human IL-2, at a concentration of 10 IU/mL to 500 IU/mL, 50 IU/mL to 250 IU/mL or 100 IU/mL to 200 IU/mL; or at a concentration of at least 10 IU/mL, 50 IU/mL, 100 IU/mL, 200 IU/mL, 300 IU/mL, IU/mL, or 500 IU/mL; and/or
the input composition comprises recombinant IL-15, optionally recombinant human IL-15, at a concentration of 1 IU/mL to 100 IU/mL, 2 IU/mL to 50 IU/mL, or 5 IU/mL to 10 IU/mL; or at a concentration of at least 1 IU/mL, 2 IU/mL, 5 IU/mL, 10 IU/mL, 25 IU/mL, or 50 IU/mL; and/or
the input composition comprises recombinant IL-7, optionally recombinant human IL-7, at a concentration of 50 IU/mL to 1500 IU/mL, 100 IU/mL to 1000 IU/mL to 200 IU/mL to 600 IU/mL; or at a concentration of at least 50 IU/mL, 100 IU/mL, 200 IU/mL, 300 IU/mL, 400 IU/mL, 500 IU/mL, 600 IU/mL, 700 IU/mL, 800 IU/mL, 900 IU/mL, or 1000 IU/mL.

27. The method of claim 2, wherein:
(1) the harvested output composition is washed to obtain the cells transduced with recombinant nucleic acid;
(2) the cells transduced with the recombinant nucleic acid are added to a buffer for preservation; and preferably, the buffer contains a cell cryopreservation agent; or
(3) the cells transduced with the recombinant nucleic acid are harvested and administerrd to a subject in need thereof without in vitro expansion.

28-29. (canceled)

30. A composition of the cells transduced with the recombinant nucleic acid produced by the method of claim 2.

31. The composition of the cells transduced with the recombinant nucleic acid of claim 30, wherein the cells are immune effector cells; preferably the cells are T cells.

32. (canceled)

33. The composition of the cells transduced with the recombinant nucleic acid of claim 31, wherein:
(1) the proportion of TSCMs in the cells transduced with the recombinant nucleic acid is higher than the proportion of TSCMs in the cells to be transduced;
preferably, the proportion of TSCMs in the cells transduced with the recombinant nucleic acid is about 2 times or more the proportion of TSCMs in the cells to be transduced;
and more preferably, the proportion of TSCMs in the cells transduced with the recombinant nucleic acid is about 3 times or more the proportion of TSCM in the cells to be transduced;
(2) the proportion of TSCMs in the cells transduced with the recombinant nucleic acid is 10% or higher, preferably 13% or higher, and more preferably 15% or higher; or
(3) the cells transduced with the recombinant nucleic acid are administered to a subject without in vitro expansion.

34-37. (canceled)


SEQUENCE LISTING (4 sequences)

SEQ ID NO: 1 - DNA, 1470 bp, Artificial sequence
  Synthesized polynucleotide
atggccttaccagtgaccgccttgctcctgccgctggccttgctgctccacgccgccaggccggaggtgcaattgctggagtctgggggaggcttggtacagcctggggggtccctgagactctcctgtgcagcctccggattcacctttggcggtaatgccatgtcctgggtccgccaggctccagggaaggggctggagtgggtctcagcaattagtggtaatggtggtagtacattctacgcagactccgtgaagggccggttcaccatctccagagacaattccaagaacacgctgtatctgcagatgaacagcctgagagccgaggacacggccgtatattactgtgcgaaagttcgtccattctggggtactttcgactactggggccaaggaaccctggtcaccgtctcgagtggtggaggcggttcaggcggaggtggttctggcggtggcggatcggaaatcgtgttaacgcagtctccaggcaccctgtctttgtctccaggggaaagagccaccctctcttgcagggccagtcagagtgttagcagcagctacttagcctggtaccagcagaaacctggccaggctcccaggctcctcatctatggagcatccagcagggccactggcatcccagacaggttcagtggcagtggatccgggacagacttcactctcaccatcagcagactggagcctgaagattttgcagtgtattactgtcagcagtacttcaacccaccagaatacacgttcggccaggggaccaaagtggaaatcaaacgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 2 - protein, 489 aa, Artificial sequence
  Synthesized polypeptide
MALPVTALLLPLALLLHAARPEVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 3 - protein, 490 aa, Artificial sequence
  Synthesized polypeptide
MALPVTALLLPLALLLHAARPEVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 4 - protein, 495 aa, Artificial sequence
  Synthesized polypeptide
MALPVTALLLPLALLLHAARPQVQLVQSGAEVKKPGSSVKVSCKASGYTFSSYWMNWVRQAPGQGLEWMGQIWPGDGDTNYNGKFKGRVTITADESTSTAYMELSSLRSEDTAVYYCARRETTTVGRYYYAMDYWGQGTTVTVSSGGGGSGGGGSGGGGSDIVLTQSPASLAVSPGQRATITCKASQSVDYDGDSYLNWYQQKPGQPPKLLIYDASNLVSGVPARFSGSGSGTDFTLTINPVEANDTANYYCQQSTEDPWTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR